(19) United States
(12) Patent Application Publication
(10) Pub. No.: US 2023/0131993 A1
(43) Pub. Date: Apr. 27, 2023

(54) COMPOSITION COMPRISING AN AVENANTHRAMIDE OR AN ANALOGUE THEREOF WITH IMPROVED STABILITY

(71) Applicant: SYMRISE AG, Holzminden (DE)

(72) Inventors: Sabine Lange, Holzminden (DE); Martina Herrmann, Hameln (DE); Sebastian Bruncke, Höxter (DE); Holger Joppe, Dassel (DE); Kalpana Palani, Holzminden (DE)

(21) Appl. No.: 17/909,342

(22) PCT Filed: Mar. 6, 2020
(86) PCT No.: PCT/EP2020/056122

§ 371 Date: Sep. 2, 2022

(51) Int. Cl. A61K 31/196; A61K 36/899; A61K 47/12; A61K 47/10; A61K 47/08; A61K 8/9794; A61Q 17/04; A61Q 19/00

(52) U.S. Cl. CPC A61K 31/196; A61K 36/899; A61K 47/12; A61K 47/10; A61K 47/08; A61K 8/9794; A61Q 17/04; A61Q 19/00; A61K 2800/52

(57) ABSTRACT

The present invention relates generally to: a composition comprising or consisting at least one avenanthramide or an analogue thereof or an oat extract comprising an avenanthramide or an analogue thereof with an improved stability; its cosmetic or medical use; the use of such composition for the preparation of foods, food supplements, cosmetic, pharmaceutical or veterinary preparations; and foods, food supplements, cosmetic, pharmaceutical or veterinary preparations comprising such a composition. Finally, the present invention relates to specific stabilizers for the stabilisation of avenanthramide(s).

### TECHNICAL FIELD

The present invention relates generally to: a composition comprising or consisting at least one avenanthramide or an analogue thereof or an oat extract comprising an avenanthramide or an analogue thereof with an improved stability; its cosmetic or medical use; the use of such composition for the preparation of foods, food supplements, cosmetic, pharmaceutical or veterinary preparations; and foods, food supplements, cosmetic, pharmaceutical or veterinary preparations comprising such a composition. Finally, the present invention relates to specific stabilizers for the stabilisation of avenanthramide(s).

### BACKGROUND ART

Poaceae, also known as Gramineae, is a large and nearly ubiquitous family of monocotyledonous flowering plants known as grasses. Grasses are an economically important family of plants. They have been grown as feed for domesticated animals for up to 6,000 years, and the grains of grasses such as wheat, rice, maize (corn), barley, sorghum, millet and oat have been and still are the most important human food crops.

Cereal grains are an excellent source of numerous unique substances among biologically active compounds such as dietary fibre (arabinoxylans, β-glucans, cellulose, lignin and lignans), sterols, tocopherols, tocotrienols, phenolic compounds, vitamins and microelements.

Oatmeal has been used for centuries as a soothing agent to relieve itching and irritation associated with various xerotic dermatoses. Medical texts promoted the topical application of oatmeal flour for a variety of dermatological conditions. The most common clinical applications for colloidal oatmeal in dermatological practice are as an adjunctive therapy for pruritic skin conditions such as atopic dermatitis and allergic or irritant contact dermatitis. The direct anti-irritant activity of oats has been well established both in vitro and in clinical studies. Extracts of oats have been shown to decrease the ionophore-stimulated liberation of arachidonic acid from phospholipids in keratinocytes and inhibit prostaglandin biosynthesis. Despite the wide-spread use of skin anti-irritants, few studies have examined the phytochemicals present in oats that mediate the anti-inflammatory activity.

Oats exist in two main species, Avena sativa L. and Avena nuda L. (synonyms include Avena sativa subsp. nuda (L.) after Gillet & Magne, and Avena sativa var. nuda (L.) after Körn). A. sativa, also known as common or husked oat, is primarily grown in cool temperate climates, in particular in the cool and moist regions of Northern Europe and North America. A. nuda is known as naked or huskless oat because the husk is removed when the crop is harvested, and it has a free threshing character similar to wheat. Husked oats represent the majority of global oat production, except in China, where naked oat is the most common type.

The composition of oats is predominantly starch (65 to 85%), proteins (15 to 20%, including enzymes), lipids (3 to 11%) and about 2 to 8.5% dietary fibres including a high content of beta-glucans. Oats also contain other important bioactive compounds such as phenolic compounds.

A general definition of a phenolic compound is any compound containing a benzene ring with one or more hydroxyl groups. Phenolic acids, flavonoids, condensed tannins, coumarins and alkylresorcinols are examples. In cereal grains, these compounds are located mainly in the pericarp, and they can be concentrated by decorticating the grain to produce bran. Phenolic compounds can be grouped into flavonoids (sub-classified as flavonols, flavones, isoflavones, anthocyanins, flavanols, flavanones, etc.) and non-flavonoids. Phenolic compounds can exist as free phenols or in glycosidic form. They tend to be relatively polar and typically dissolve in pure or aqueous alcohols such as ethanol and methanol or aqueous acetone. Many phenolic compounds in cereals (such as phenolic acids and flavonoids) are also reported in fruits and vegetables, but some phenols are unique to one plant species, such as for example oat avenanthramides. Phenolic compounds have been shown to possess numerous activities, the most important being the antioxidant activity which prevents lipid peroxidation and cellular oxidative damage mediated by harmful free radicals. This property is related to the ability of phenolic compounds to scavenge free radicals, donate hydrogen atoms or electrons, or chelate metal cations [Dykes et al., Cereal Foods World, 2007, 105-111]. Phenolic compounds have antioxidant properties and, thus, can protect against degenerative diseases (such as heart disease and cancer) in which reactive oxygen species (i.e. superoxide anions, hydroxyl radicals and peroxy radicals) are involved.

The type and concentration of phenolic compounds in wholemeal cereals are influenced by the plant variety and nature of the grain. Besides containing high levels of phenolic acids, tocopherols and alk(en)ylresorcinol derivatives, oats are in particular a unique source of avenanthramides (Avns; also known as N-cinnamoyl anthranilate alkaloids or anthranilic acid amides), which are not present in other cereals. These compounds are antipathogens, which are produced by the plant in response to the exposure to pathogens such as fungus.

Avenanthramides (in the following abbreviated as Avns or Avn for a single avenanthramide compound), which are low-molecular-weight phenolic amides containing anthranilic acid and hydroxycinnamic acid moieties with an amide bond, are a group of naturally occurring phenolic amides in oats, both A. sativa and A. nuda. They were originally identified as phytoalexins produced by the plant in response to exposure to pathogens, such as fungi. Oats contain a unique group of approximately 40 different types of Avns, which are present in both oat grains and leaves. The most abundant are Avn A (N-(4′-hydroxycinnamoyl)-5-hydroxyanthranilic acid), Avn B (N-(4′-hydroxy-3′-methoxycinnamoyl)-5-hydroxyanthranilic acid) and Avn C (N-(3′-4′-dihydroxycinnamoyl)-5-hydroxyanthranilic acid), which are amides of 5-hydroxyanthranilic acid with p-coumaric, ferulic and caffeic hydroxycinnamic acids, respectively. These Avns are constitutively expressed in the kernels, appearing in almost all milling fractions, but occur at their highest concentrations in the bran and outer layers of the kernel [Boz H, Czech Journal of Food Sciences 2015, 33(5): 399-404]. The total content of avenanthramides (Avns) in oat grain has been found to be about 2 to 700 mg/kg (0.0002 to 0.07%), depending on the cultivar and agronomic treatment [Maliarova M et al., Journal of the Brazilian Chemical Society 2015, 26(11), 2369-2378].

The extraction of Avns from oats was carried out using various solvent compositions such as pure or diluted ethanol and methanol. Extraction procedures were achieved over different times at room temperature or under controlled heating, such as naked oats, 50% aqueous ethanol [Tong L et al., Journal of Integrative Agriculture 2014, 13, 1809].

Maliarova, M. et al., Journal of the Brazilian Chemical Society 2015, 26(11), 2369-2378 compared the efficiency of methanol, ethanol and isopropanol on the extraction of Avns from naked oat bran. The optimum conditions for the highest yield of Avns were a methanol concentration of 70%, an extraction temperature of 55° C. and an extraction time of 165 minutes.

The application of avenanthramides is a growing field in therapeutics since a number of studies have demonstrated that avenanthramides have excellent antioxidant activity both in vitro and in vivo, as well as anti-inflammatory, anti-irritant, anti-atherogenic and anti-proliferative activities which may prevent or limit cellular oxidative dysfunctions and the development of oxidative stress-related diseases, such as neurodegenerative and cardiovascular diseases, and provide additional protection against skin irritation, aging, CHD and cancer [Perrelli A et al., Oxidative Medicine and Cellular Longevity 2018, DOI: 10.1155/2018/6015351].

The antioxidant activity of Avns has been found to be 10 to 30 times higher than those of the typical cereal components ferulic acid, gentisic acid, phydroxybenzoic acid, protocagtechuic acid, syringic acid, vanillic acid and vanillin. The Avns differ in the antioxidant activity, Avn C having the highest activity, followed by Avn B and Avn A. Avns enriched oat extracts inhibit LDL oxidation in vitro. Both, animal studies and human clinical trials confirmed that oats antioxidants have the potential of reducing cardiovascular risks by lowering serum cholesterol, inhibiting LDL cholesterol oxidation and peroxidation. Another study has indicated that the consumption of oats and oats bran may reduce the risk of colon cancer not only because of their high fiber contents but also due to Avns. Furthermore, Avns enriched oat extracts have been shown to inhibit atherosclerosis and activation of the NF-kB transcription factor, which is the regulator of infection and inflammation [Hüseyin Boz, Phenolic Amides (Avenanthramides) in Oats—A Review, Czech J. Food Sci., 33, 2015 (5), 399-404].

WO 2004/047833 A1 describes the inhibition of substance P-induced liberation of histamine from mast cells and the treatment and prevention of itching by substances of the above Formula 2.

WO 2017/159964 A1 describes compositions comprising, as an active ingredient, avenanthramide or a derivative thereof, for preventing or treating hearing loss.

EP 0 157 420 A2 describes avenanthramides, including compounds structurally related to avenanthramide L, as 5-lipoxygenase inhibitors.

Lotts T et al., Experimental Dermatology 2017, 26(8): 739-742, describes how dihydroavenanthramide D (CAS 697235-49-7, INCI name: hydroxyphenyl propamidobenzoic acid; the active ingredient in SymCalmin® provided by Symrise) inhibits mast cell degranulation and exhibits anti-inflammatory effects through the interaction with the neurokinin-1 receptor.

Cereal phenolc compounds, particularly avenanthramides, have potent beneficial biological activities, making them valuable and highly interesting naturally active ingredients for nutritional, cosmetic and health use for oral and/or topical applications for humans and animals.

There is thus an ongoing need and consumer demand in the food, cosmetic, pharmaceutical and veterinary industry for the development of new formulations based on naturally occurring, well tolerated and biodegradable substances, especially for use in skin protection and in the prevention and/or treatment of dermatoses, which are stable and do not degrade during processing steps, storage or transport as well as by the influence of light and UV irradiation, metal cations, air or certain enzymes, respectively.

Oxidative degradation processes and autoxidative processes play an important role for the stability of preparations, since they can destroy desired ingredients that then are not present, can produce unpleasant or undesired degradation products or adversely influence physical performance characteristics of the product, for example color, and, hence, often decreases the value of the product.

Avenanthramides, that find application in the nutrition, cosmetic, pharmaceutical or veterinary industry, however, are instable compounds due to their chemical structure. The antioxidant activity of phenolic acids is related to the number and position of hydroxyl groups in the molecule. The antioxidant efficiency of mono-phenols is strongly enhanced by the introduction of a second hydroxyl group at the ortho or para positions, and is increased by one or two methoxy substituents in ortho position with respect to the hydroxyl group. The avenanthramide compounds therefore reduce or inhibit the oxidation of other compounds; by doing so, they get oxidized themselves, i.e. they degrade in the course of time, which is disadvantageous for the composition or the final product, since the avenanthramides as active substances or active ingredients are no longer present or only present in a minor concentration.

Among the avenanthramide compounds, especially Avenanthramide C is known to have a low stability and leads to quickly destabilization during storage.

Hence, in order to maintain the activity of avenanthramides as active substance or active ingredient, and to extend the shelf life of compositions or preparations comprising avenanthramide compounds, the compositions or preparations are to be stabilized.

In order to stabilise a composition comprising one avenanthramide or a mixture of avenanthramides, vitamin C (ascorbic acid) has been used until now. However, the use of higher concentrations of vitamin C (ascorbic acid) leads to unpleasant discoloration of the composition, and, thus decreases the value of the product. Especially in end formulations such as emulsions, the stabilisation with vitamin C (ascorbin acid) results in undesirable discoloration. The use of other avenanthramide stabilizing agents is not yet known.

Achieving therapeutic concentrations or bioavailability of the avenanthramide substances is therefore highly challenging.

Accordingly, it is the object of the present invention to provide a composition comprising an avenanthramide or an analogue thereof or an oat extract comprising an avenanthramide or an analogue thereof which exhibits enhanced stability during storage, while the stabilisation does not lead to discoloration.

In particular, the aim of the present invention is to suggest naturally, biodegradable, cosmetically or pharmaceutically well tolerated, safe, easy-to-use and stable stabilizer substances which are able to stabilize avenanthramide in a composition or in an oat extract and which do not interfere with the beneficial biological activity of avenanthramides and the formulation properties as such. Especially, the aim of the present invention is to suggest naturally, biodegradable, cosmetically or pharmaceutically well tolerated, safe, easy-to-use and stable stabilizer substances which are able to stabilize avenanthramide compounds, especially avenanthramides A, B, C or L or the avenanthramide analogue compound Dihydroavenanthramide D (2-{[3-(4-Hydroxyphenyl)propanoyl]amino}benzoic acid; INCI: Hydroxyphenyl Propamidobenzoic Acid; CAS 697235-49-7) in a composition or in an oat extract and do not lead to discoloration.

Surprisingly, it turns out that avenanthramides, in particular avenanthramide C, which is the most unstable among the avenanthramide, can be significantly stabilized, by a stabilizer, in particular by at least one stabilizer which is a chelator or an organic acid or an antioxidant or mixtures thereof, particularly at a low concentration. For some of said stabilizer combinations even a synergistic effect can be demonstrated. This is particularly the case, wherein the at least one avenanthramide analogue is Dihydroavenanthramide D (2-{[3-(4-Hydroxyphenyl)propanoyl]amino}benzoic acid).

### SUMMARY OF THE INVENTION

The aforementioned object is achieved in accordance with a first aspect of the present invention by providing a composition comprising or consisting of:
(i) at least one avenanthramide or an analogue thereof, or an oat extract comprising at least one avenanthramide or an analogue thereof; and
(ii) at least one stabilizer selected from the group consisting of chelating agents, organic carbonic acids, antioxidants and mixtures thereof.

In a second aspect, the present invention relates to the use of said composition as a cosmetic, in particular for use as a dermatological cosmetic in skin protection and skin care, scalp protection and scalp care, hair care, nail care or in the prevention and/or treatment of skin conditions, intolerant and sensitive skin, skin irritation, skin reddening, wheals, pruritus (itching), skin aging, wrinkle formation, loss of skin volume, loss of skin elasticity, pigment spots, pigment abnormalities, or dry skin, i.e. for moisturising the skin.

In a third aspect, the present invention relates to the use of said composition as a medicament, in particular for use in the prevention and/or treatment of dermatological or keratological diseases, in particular of dermatological or keratological diseases having a barrier related, inflammatory, immunoallergic, atherogenic, xerotic or hyperproliferative component or in the prevention and/or treatment of dermatological diseases associated with increased ROS production or in the prevention and/or treatment of cardiovascular diseases, allergic reactions, coronary heart disease, for decreasing the level of LDL cholesterol and lipids in blood serum, for reducing blood pressure, for improving sensitivity to insulin and for enabling the control of blood glucose levels.

In a fourth aspect, the present invention relates to the use of said composition for preparing foods, food supplements, cosmetic, pharmaceutical and veterinary preparations.

In a fifth aspect, the present invention relates to foods, food supplements, cosmetic, pharmaceutical and veterinary preparations comprising the composition according to the present invention.

Finally, the present invention relates to the use of one or more of said stabilizers for the stabilization of an avenanthramide or an analogue avenanthramide compound.

The invention is specified in the appended claims. The invention itself, and its preferred variants, other objects and advantages, are however also apparent from the following detailed description in conjunction with the accompanying examples.

### DETAILED DESCRIPTION OF THE INVENTION

In a first aspect, the present invention relates to a composition comprising or consisting of:
at least one avenanthramide or an analogue thereof, or an oat extract comprising at least one avenanthramide or an analogue thereof; and
at least one stabilizer, selected from the group consisting of chelating agents, organic acids, antioxidants and mixtures thereof.

The first main ingredient of the composition according to the first aspect of the present invention is at least one avenanthramide or an analogue thereof, or an oat extract comprising at least one avenanthramide or an analogue thereof.

As used in this document, the phrase “at least one of” means that the composition or the oat extract can comprise for example either one avenanthramide or more than one avenanthramide. Additionally, the phrase “at least one of”, when applied to a list, means anyone combination of the items specified in the list.

The composition according to the first aspect of the present invention is prepared by combining the ingredients specified, as described in further detail below.

Within the context of the present invention, the term “avenanthramide(s)” (anthranilic acid amides) is understood to mean a member of a group of phenolic alkaloids, i.e. naturally occurring avenanthramide(s), found mainly in oats (Avena sativa) but also present in white cabbage butterfly eggs (Pieris brassicae and P. rapae) and in fungus-infected carnations (Dianthus caryophyllus), as described in detail hereinafter, or non-naturally occurring artificial produced avenanthramide analogue(s), as described in detail hereinafter.

The avenanthramides of the composition of the present invention are naturally found in and can be isolated and purified from oats. The two main species of oats are Avena sativa L. and Avena nuda L. (synonyms include Avena sativa subsp. nuda (L.) after Gillet & Magne, and Avena sativa var. nuda (L.) after Körn). A. sativa is also known as common or husked oat. A. nuda is known as naked or huskless oat because the husk is removed when the crop is harvested. Oats can be processed and separated into constituent fractions including oat grains, wherein they appear to be most concentrated in the peripheral regions, husks, trichomes or straw. More than 50 distinct avenanthramides have been isolated from oat grains [Collins, Journal of Agricultural and Food Chemistry, 37 (1989), 60-66].

Avns can be represented by the following general Formula 1:

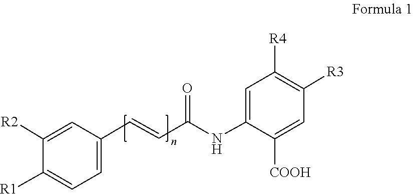

The following Table 1 shows examples of naturally occurring Avns based on general Formula 1.

TABLE 1

| Avenanthramide *) | CAS number | N | R1 | R2 | R3 | R4 |
|---|---|---|---|---|---|---|
| A | 108605-70-5 | 1 | OH | H | OH | H |
| B | 108605-69-2 | 1 | OH | OMe | OH | H |
| C | 116764-15-9 | 1 | OH | OH | OH | H |
| D | 115610-36-1 | 1 | OH | H | H | H |
| E | 93755-77-2 | 1 | OH | OMe | H | H |
| F | 116764-16-0 | 1 | OH | OH | H | H |
| G | 116764-17-1 | 1 | OH | H | H | OH |
| H | 116764-18-2 | 1 | OH | OMe | H | OH |
| K | 116764-19-3 | 1 | OH | OH | H | OH |
| X | 1158480-77-3 | 1 | OH | H | OH | OMe |
| Y (2 **) | 154992-25-3 | 1 | OH | OMe | OH | OMe |
| Z | 1158480-80-8 | 1 | OH | OH | OH | OMe |
| AA | 157799-28-5 | 1 | OH | H | OH | OH |
| BB | 2304718-64-5 | 1 | OH | OMe | OH | OH |
| CC | 1819995-77-1 | 1 | OH | OH | OH | OH |
| O(L **) | 172549-38-1 | 2 | OH | H | OH | H |
| P | 1358438-37-5 | 2 | OH | OMe | OH | H |
| Q | 2227208-43-5 | 2 | OH | OH | OH | H |
| L | 2301866-39-5 | 2 | OH | H | H | H |
| M | 101618-11-5 | 2 | OH | OMe | H | H |
| N | 101618-21-7 | 2 | OH | OH | H | H |
| R | 1191042-39-3 | 2 | OH | H | H | OH |
| S | 2301866-43-1 | 2 | OH | OMe | H | OH |
| T | 2301864-63-9 | 2 | OH | OH | H | OH |
| U | 2301864-86-6 | 2 | OH | H | OH | OMe |
| V | 2304718-63-4 | 2 | OH | OMe | OH | OMe |
| W | 2304718-62-3 | 2 | OH | OH | OH | OMe |
| OO | 2301866-28-2 | 2 | OH | H | OH | OH |
| PP | 2301864-57-1 | 2 | OH | OMe | OH | OH |
| QQ | 2301864-89-9 | 2 | OH | OH | OH | OH |

*) Abbreviations Collins [de Bruijn et al., Food Chemistry (2018), doi: https://doi.org/10.1016/j.foodchem.2018.11.013, supplementary information Table S1]
**) More commonly used, non-Collins abbreviations

A number of studies have demonstrated that these natural products have anti-inflammatory, anti-oxidant, anti-itch, anti-irritant and anti-atherogenic activities.

The naturally occurring avenanthramides or mixtures of avenanthramides as described above, can be obtained, enriched and isolated from the plant of the genus Avena by extraction, in particular from any oat species, fresh or dried, or parts thereof, such as milled grains, non-milled grains, husks, trichomes or oat straw of the species Avena sativa or Avena nuda.

The extracting solvent (extractant) for favourably extracting the avenanthramide L is selected from the group consisting of mixtures of water and an organic solvent, wherein the organic solvent is preferably a solvent suitable for foodstuffs or cosmetic or pharmaceutical preparations. It goes without saying that such solvents need be suitable for and compatible with the preparation of foods, cosmetics or pharmaceutical preparations.

In a more preferred variant, the extracting solvent comprises a mixture of water and an alcohol or acetone. The alcohol is preferably selected from the group consisting of methanol, ethanol, n-propanol, isopropanol, n-butanol, isobutanol, t-butanol and mixtures, i.e. combinations, thereof. The most preferred extracting solvents (extractant) for the extraction step of the present invention are methanol, ethanol, n-propanol, isopropanol or acetone or any mixtures respective combinations of said solvents, each in mixture with water. The use of pure organic solvents is not advantageous, due to the co-extraction of triglycerides.

The mixing ratio of water to the organic solvent, preferably water to the alcohol or water to acetone, in the extracting solvent is in a range of 10:90 to 90:10 (v/v), preferably in a range of 20:80 to 80:20 (v/v) and most preferably in a range of 30:70 to 70:30 (v/v), based in each case on the resulting extracting solvent.

Particularly preferred extracting solvents (extractants) are: methanol/water (3:7), methanol/water (1:1), methanol/water (7:3), ethanol/water (3:7), ethanol/water (1:1), ethanol/water (1:4), ethanol/water (7:3), isopropanol/water (3:7), isopropanol/water (1:1), isopropanol/water (7:3), aceton/water (3:7), aceton/water (1:1), aceton/water (7:3).

In order to improve the extraction yield, the oat source is extracted at a temperature ranging from 30 to 80° C., preferably from 40 to 70° C. and more preferably from 50 to 60° C. The extraction yield for milled oat grains increases with increasing temperatures between 40 and 70° C.

Apart from avenanthramide compounds enriched, isolated and purified from natural sources, the naturally occurring avenanthramides can be produced by organic synthesis. Methods of synthesis known in the art are illustrated for example in U.S. Pat. Nos. 6,096,770 and 6,127,392, Japanese Patent No. J60019 754 A and Hungarian Patent No. HU 200 996 B.

Said synthetic prepared avenanthramide substances are identical to the corresponding naturally occurring avenanthramide compounds as isolated or extracted from oats.

The non-naturally occurring avenanthramide analogue(s) in the composition according to the present invention, hereinafter also referred to as “analogue(s)” or “analogue avenanthramide compound(s)” which are in accordance with the following Formula 2 and endowed with important biological properties have been artificially produced by organic synthesis methodologies, such as for example those given in WO 2004/047833 A1 or WO 2007/062957 A1:

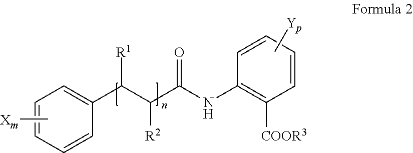

where m=0, 1, 2 or 3, p=0, 1 or 2, and n=0, 1 or 2,
with the proviso that if n=1 or 2, then p+m>0,
and if n=1 or 2, then R¹ and R², in respective pairs, respectively denote H or together denote another chemical bond (as for example in cinnamic acid derivatives),
and if m=1, 2 or 3, then each X independently denotes OH, Oalkyl or Oacyl,
and if p=1 or 2, then each Y independently denotes OH, Oalkyl or Oacyl,
and if p+m>0, then at least one of X and Y is selected from the group consisting of OH and Oacyl,
and where R³ is —H or an alkyl (in particular —CH₃, or other straight-chain or branched alkyl chains with 2 to 30 C atoms; in this context, R³ is also —H for the corresponding pharmaceutically acceptable salts).

Particularly preferred compounds of Formula 2 according to the invention are those in which:

n=1 or 2 and p+m>0; and/or
p+m>0 and X or Y at least one of X and Y is selected from the group consisting of OH and Oalkyl.

Particularly preferably, a compound of Formula 2 is used in which n=1 and p+m>2, with the proviso that at least two of X and Y are together selected from the group comprising OH and Oalkyl.

It is also preferable to use a compound of Formula 2 in which n=1 and m=1, 2 or 3, with the proviso that at least one X is selected from the group comprising OH and Oalkyl, and/or P=1 or 2, with the proviso that at least one Y is selected from the group comprising OH and Oalkyl.

If n has the value 1, then R¹ and R² are each preferably H, although it is also possible for R¹ and R² together to be another chemical bond.

With regard to the definition of Formula 2 and the specific avenanthramide compounds disclosed in WO 2004/047833 A1 or WO 2007/062957 A1, the corresponding disclosure in said documents is hereby incorporated by reference.

The avenanthramide analogue compound of Formula 2 is preferably selected from the group consisting of:

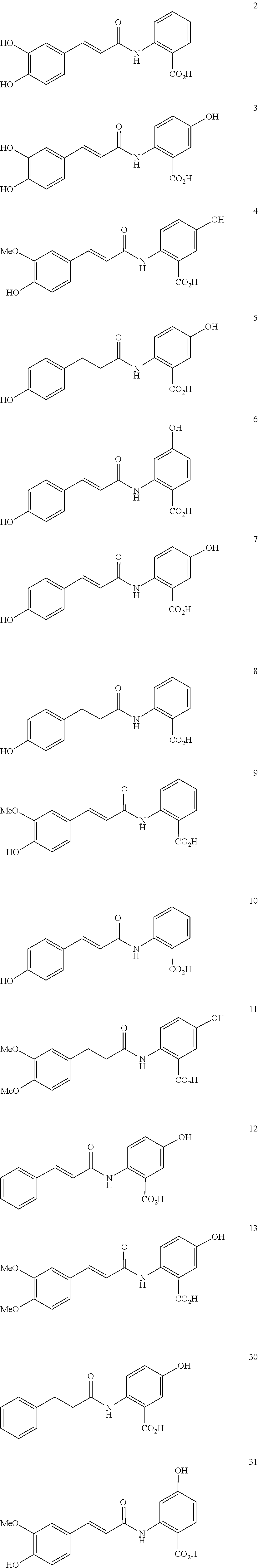

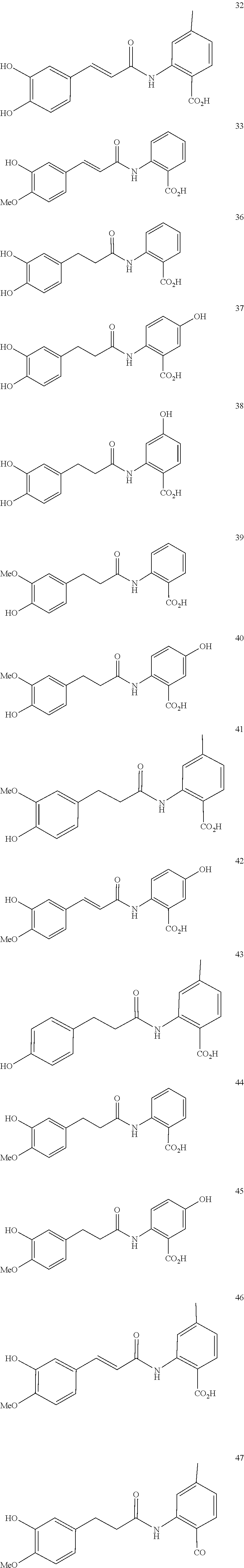

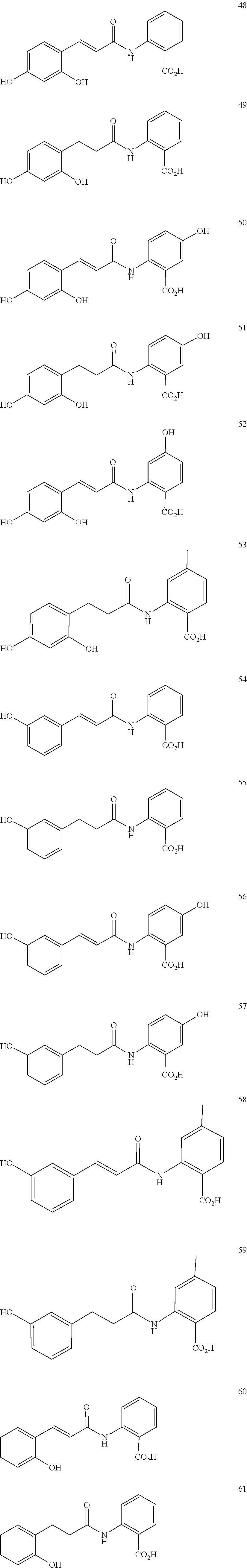

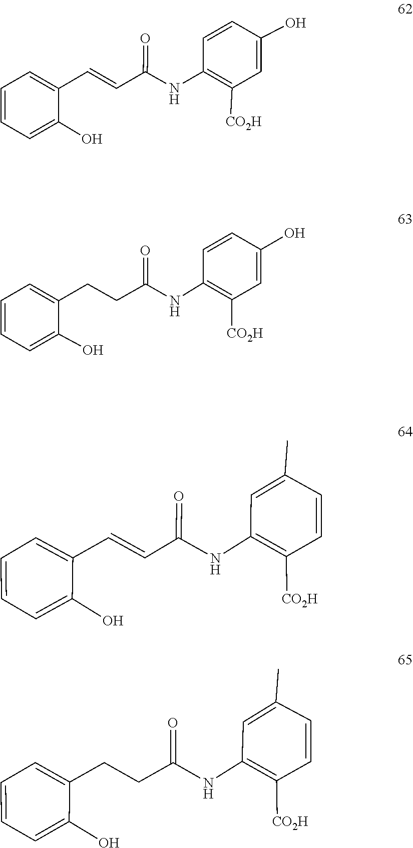 [SMILES: O=C(/C=C/c1ccccc1O)Nc1ccc(O)cc1C(=O)O ; O=C(CCc1ccccc1O)Nc1ccc(O)cc1C(=O)O ; Cc1ccc(C(=O)O)c(NC(=O)/C=C/c2ccccc2O)c1 ; Cc1ccc(C(=O)O)c(NC(=O)CCc2ccccc2O)c1]

The above illustrations relate essentially to compounds of Formula 2 in which n=1.

However, the use of compounds of Formula 2 in which n=0 is also frequently preferred, in which case it preferably holds that m+p=0, or m+p>1 or 2, with the proviso that at least two of the substituents X and Y are selected from the group comprising OH and Oalkyl.

It is particularly preferable to use compounds of Formula 2 (where n=0) selected from the group comprising:

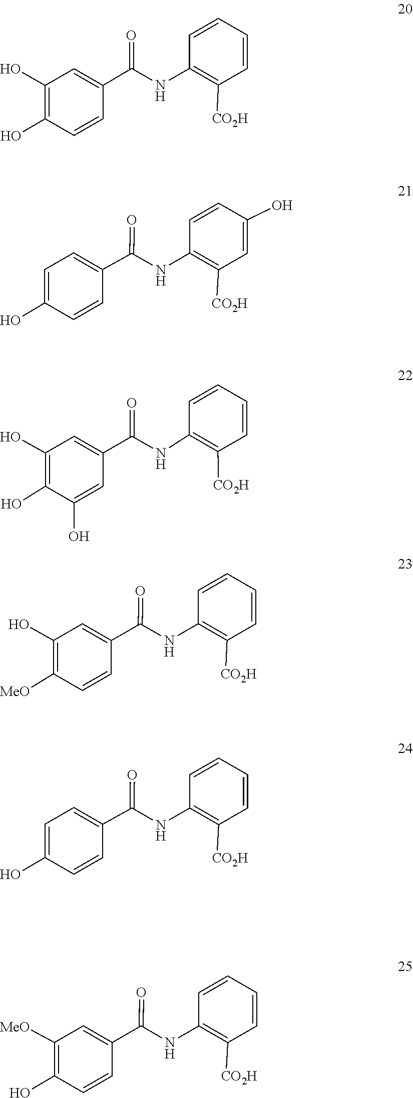 [SMILES: O=C(Nc1ccccc1C(=O)O)c1ccc(O)c(O)c1 ; O=C(Nc1ccc(O)cc1C(=O)O)c1ccc(O)cc1 ; O=C(Nc1ccccc1C(=O)O)c1cc(O)c(O)c(O)c1 ; COc1ccc(C(=O)Nc2ccccc2C(=O)O)cc1O ; O=C(Nc1ccccc1C(=O)O)c1ccc(O)cc1 ; COc1cc(C(=O)Nc2ccccc2C(=O)O)ccc1O]

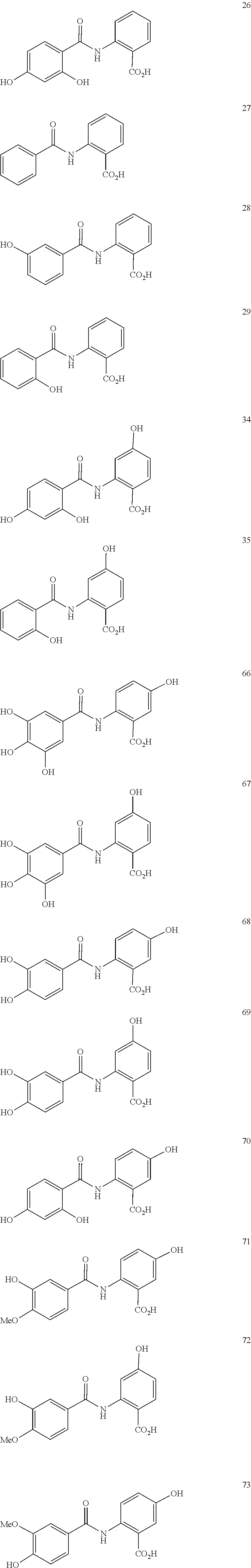

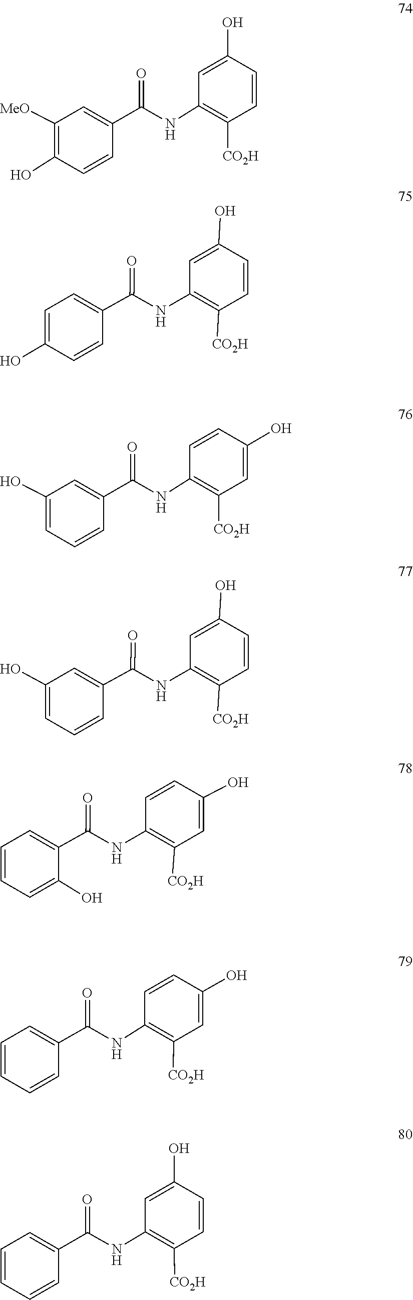

In the compounds described as particularly preferred and indicated by their structural formulae, R³ is always H.

Instead of these preferred compounds, it is also preferable in each case to use the corresponding compounds in which R³ is CH₃ or a linear or branched alkyl having 2 to 30 C atoms.

From the above avenanthramide analogue compounds compound No. 8 (Dihydroavenanthramide D) is particularly preferred.

Besides the above natural occurring avenanthramides and non-natural occurring avenanthramides analogues, novel avenanthramide analogues have been produced in recombinant yeast, including N-(4′-hydroxycinnamoyl)-3-hydroxyanthranilic acid (YAvn I) and N-(3′-4′-dihydroxycinnamoyl)-3-hydroxyanthranilic acid (YAvn II), which were generated by engineering a Saccharomyces cerevisiae strain with two plant genes (4cl-2 from tobacco and hct from globe artichoke) encoding key proteins involved in the biosynthesis of phenolic esters. Remarkably, YAvn I and YAvn II share structural similarities with Avn A and Avn C, respectively.

In the context of the present invention avenanthramides naturally occurring obtained from naturally sources or avenanthramides produced synthetically are preferred and are used likewise.

The term “avenanthramide or an analogue thereof” is intended to also include their various isomers that exist, notably the naturally occurring trans-isomers as well as the cis-isomers, such as avenanthramides with cis-isomerized double bond (Formula 1 or 2 with n=1) or 1 or 2 cis-isomerized double bonds (Formula 1 or 2 with n=2) induced e.g. by photoisomerization due to light exposure.

In particular, within the context of the present invention, the avenanthramide is any one of the avenanthramide compounds represented by the general Formula 1 and defined in Table 1 or any isomer thereof or the avenanthramide analogue is any one of the avenanthramide analogue compounds represented by the general Formula 2 and its definition or any isomer thereof as described above.

In a preferred variant of the present invention according to the first aspect, the composition comprises at least one avenanthramide selected from the group consisting of avenanthramides A, B, C, G, H, K, L and R, still more preferred avenanthramides A, B, C or L.

In another variant, the composition comprises a mixture of two, three, four or even more avenanthramides selected from the group consisting of avenanthramides A, B, C, G, H, K, L (non-Collins abbreviations; CAS number 172549-38-1) (also called 0 or 2pd) and R. The mixtures of avenanthramides can thus include any one of the following combinations of avenanthramides: A/B; A/C; A/G; A/H; A/K; A/L; A/R; B/C; B/G; B/H; B/K; B/L; B/R; C/G; C/H; C/K; C/L; C/R; G/H; G/K; G/L; G/R; H/K; H/L; H/R; K/L; K/R; and L/R; A/B/C; A/B/G; A/B/H; A/B/K; A/B/L; A/B/R; A/C/G; A/C/H; A/C/K; A/C/L; A/C/R; A/G/H; A/G/K; A/G/L; A/G/R; A/H/K; A/H/L; A/H/R; A/K/L; A/K/R; A/L/R; B/C/G; B/C/H; B/C/K, B/C/L; B/C/R; C/G/H; C/G/K, C/G/L; C/G/R, G/H/K; G/H/L; G/H/R; H/K/L, H/K/R; K/L/R; A/B/C/G; A/B/C/H; A/B/C/K; A/B/C/L; A/B/C/R; A/C/G/H; A/C/G/K; A/C/G/L; A/C/G/R; A/G/H/K; A/G/H/L; A/G/H/R; A/H/K/L; A/H/K/R; A/K/L/R; B/C/G/H; B/C/G/K; B/C/G/L; B/C/G/R; C/G/H/K; C/G/H/L; C/G/H/R; G/H/K/L; G/H/K/R and H/K/L/R.

The most preferred mixtures of avenanthramides are however A/B, A/C, A/L, B/C, B/L and A/B/C, A/B/L or A/C/L.

In addition, the composition can also comprise avenanthramides other than the avenanthramides A, B, C, G, H, K, L (non-Collins abbreviations; CAS number 172549-38-1) (also called 0 or 2pd) and R, such as avenanthramides D, E, F U, X, Y (also termed 2), AA, CC or OO or any of the remaining avenanthramide compounds of Table 1.

In another variant, the composition of the present invention comprises at least one, i.e. one, two or even more, avenanthramide(s) selected from the group consisting of avenanthramides A, B, C, G, H, K, L (non-Collins abbreviations; CAS number 172549-38-1) (also called 0 or 2pd) and R in combination with the avenanthramide analogue compound Dihydroavenanthramide D, represented by Formula 8 above. The mixtures of avenanthramides can thus include any one of the following combinations: A/Dihydroavenanthramide D; B/Dihydroavenanthramide D; C/Dihydroavenanthramide D; G/Dihydroavenanthramide D; H/Dihydroavenanthramide D; K/Dihydroavenanthramide D; L/Dihydroavenanthramide D; or R/Dihydroavenanthramide D; A/B/Dihydroavenanthramide D; A/C/Dihydroavenanthramide D; A/D/Dihydroavenanthramide D; A/G/Dihydroavenanthramide D; A/H/Dihydroavenanthramide D; A/K/Dihydroavenanthramide D; A/L/Dihydroavenanthramide D; A/R/Dihydroavenanthramide D; B/C/Dihydroavenanthramide D; B/D/Dihydroavenanthramide D; B/G/Dihydroavenanthramide D; B/H/Dihydroavenanthramide D; B/K/Dihydroavenanthramide D; B/L/Dihydroavenanthramide D; B/R/Dihydroavenanthramide D; C/D/Dihydroavenanthramide D; C/G/Dihydroavenanthramide D; C/H/Dihydroavenanthramide D; C/K/Dihydroavenanthramide D; C/L/Dihydroavenanthramide D; C/R/Dihydroavenanthramide D; G/H/Dihydroavenanthramide D; G/K/Dihydroavenanthramide D; G/L/Dihydroavenanthramide D; G/R/Dihydroavenanthramide D; H/K/Dihydroavenanthramide D; H/L/Dihydroavenanthramide D; H/R/Dihydroavenanthramide D; K/L/Dihydroavenanthramide D; K/R/Dihydroavenanthramide D; or L/R/Dihydroavenanthramide D, still more preferred A/Dihydroavenanthramide D or avenanthramide L/Dihydroavenanthramide D.

In addition to the above avenanthramide combinations, the composition can further comprise combinations with one or more avenanthramides other than the avenanthramides A, B, C, G, H, K, L (non-Collins abbreviations; CAS number 172549-38-1) (also called 0 or 2pd) and R, such as avenanthramides D, E, F U, X, Y (also termed 2), AA, CC or OO or any of the remaining avenanthramide compounds specified in Table 1 with Dihydroavenanthramide D.

In a still another variant, the composition of the present invention comprises Dihydroavenanthramide D (2-{[3-(4-Hydroxyphenyl)propanoyl]amino}benzoic acid; INCI: Hydroxyphenyl Propamidobenzoic Acid; CAS 697235-49-7) or a combination of Dihydroavenanthramide D and any one or more different avenanthramide(s) represented by general Formula 1 and specified in Table 1 as described above or an avenanthramide analogue compound(s) represented by the general Formula 2 and defined and specified above.

Alternatively to the naturally occurring avenanthramide compound(s) or non-naturally occurring avenanthramide analogue compound(s), the compostions of the invention can comprise an oat extract comprising at least one avenanthramide. Within the context of the present invention, the term “oat extract” is generally meant to encompass a compound or mixture of compounds obtained from oats by extraction.

Such extract comprising at least one avenanthramide or encompassing a mixture of avenanthramides as described above, are obtained by extraction (such as maceration, percolation, extraction by use of soxhlet, microwave or ultrasound) with water, an alcohol, acetone or mixtures thereof or by subcritical fluid extraction with these solvents or mixtures thereof. They are preferably extracted using various solvent compositions such as pure methanol, ethanol, n-propanol, isopropanol, n-butanol, isobutanol, t-butanol and mixtures, i.e. combinations, thereof or said solvents in mixture with water. Extraction procedures were achieved over different times at room temperature or under controlled heating, such as naked oats, 50% aqueous ethanol [Tong L. et al., Journal of Integrative Agriculture 2014, 13, 1809]. Maliarova, M. et al., Journal of the Brazilian Chemical Society 2015, 26(11), 2369-2378 compared the efficiency of methanol, ethanol and isopropanol on the extraction of Avns from naked oat bran. The optimum conditions for the highest yield of Avns are a methanol concentration of 70%, an extraction temperature of 55° C. and an extraction time of 165 minutes.

The extract is obtained from the plant of the genus Avena, in particular from any oat species, fresh or dried, or parts thereof, such as milled grains, non-milled grains, husks, trichomes or oat straw of the oat species Avena sativa or Avena nuda. Starting product for the extraction can also be oat grain residues from oat oil production.

In a preferred variant, the starting material for the oat extract is milled or non-milled grains of the species Avena sativa or Avena nuda or oat straw.

The extracting solvent (extractant) for favourably extracting the avenanthramide L and the naturally occurring analogue avenanthramide compounds is selected from the group consisting of mixtures of water and an organic solvent, wherein the organic solvent is preferably a solvent suitable for foodstuffs or cosmetic or pharmaceutical preparations. It goes without saying that such solvents need be suitable for and compatible with the preparation of foods, cosmetics or pharmaceutical preparations.

In a more preferred variant, the extracting solvent comprises a mixture of water and an alcohol or acetone. The alcohol is preferably selected from the group consisting of methanol, ethanol, n-propanol, isopropanol, n-butanol, isobutanol, t-butanol and mixtures, i.e. combinations, thereof. The most preferred extracting solvents (extractant) for the extraction step of the present invention are methanol, ethanol, n-propanol, isopropanol or acetone or any mixtures respective combinations of said solvents, each in mixture with water. The use of pure organic solvents is not advantageous, due to the co-extraction of triglycerides.

The mixing ratio of water to the organic solvent, preferably water to the alcohol or water to acetone, in the extracting solvent is in a range of 10:90 to 90:10 (v/v), preferably in a range of 20:80 to 80:20 (v/v) and most preferably in a range of 30:70 to 70:30 (v/v), based in each case on the resulting extracting solvent.

Particularly preferred extracting solvents (extractants) are: methanol/water (3:7), methanol/water (1:1), methanol/water (7:3), ethanol/water (3:7), ethanol/water (1:1), ethanol/water (1:4), ethanol/water (7:3), isopropanol/water (3:7), isopropanol/water (1:1), isopropanol/water (7:3), acetone/water (3:7), acetone/water (1:1), acetone/water (7:3).

From said extracting mixtures (extractants) methanol/water (1:1), methanol/water (7:3), ethanol/water (1:1), ethanol/water (1:4), isopropanol/water (3:7), isopropanol/water (1:1), isopropanol/water (7:3), acetone/water (3:7), acetone/water (1:1) and acetone/water (7:3) are particularly advantageous, since the extraction with these extractants results in an extract with high avenanthramide L content (see Table 10). The yield of avenanthramide L with these extractants is >150 ppm, more preferably >190 ppm and most preferably >200 ppm.

In order to improve the extraction yield, the oat source is extracted at a temperature ranging from 30 to 80° C., preferably from 40 to 70° C. and more preferably from 50 to 60° C. The extraction yield for milled oat grains increases with increasing temperatures between 40 and 70° C. Extracting from milled oats gives the best results in terms of yield and avenanthramide(s) content, in particular avenanthramide L content, at temperatures between 50 and 60° C., which is therefore preferred.

Altering the composition of the solvent can change the extract selectivity of the avenanthramide substances to be extracted, and thus the composition, thereby enhancing or reducing its biological activity.

The oat extract of the composition according to the first aspect of the present invention, comprises at least one avenanthramide selected from the group consisting of avenanthramides A, B, C, G, H, K, L (non-Collins abbreviations; CAS number 172549-38-1) (also called O or 2pd) and R.

In another variant, the oat extract comprises a mixture of two, three, four or even more avenanthramides selected from the group consisting of avenanthramides A, B, C, G, H, K, L (non-Collins abbreviations; CAS number 172549-38-1) (also called O or 2pd) and R. The mixtures of avenanthramides can thus include any of the following combinations of avenanthramides: A/B; A/C; A/G; A/H; A/K; A/L; A/R; B/C; B/G; B/H; B/K; B/L; B/R; C/G; C/H; C/K; C/L; C/R; G/H; G/K; G/L; G/R; H/K; H/L; H/R; K/L; K/R; and L/R; A/B/C; A/B/G; A/B/H; A/B/K; A/B/L; A/B/R; A/C/G; A/C/H; A/C/K; A/C/L; A/C/R; A/G/H; A/G/K; A/G/L; A/G/R; A/H/K; A/H/L; A/H/R; A/K/L; A/K/R; A/L/R; B/C/G; B/C/H; B/C/K, B/C/L; B/C/R; C/G/H; C/G/K, C/G/L; C/G/R, G/H/K; G/H/L; G/H/R; H/K/L, H/K/R; K/L/R; A/B/C/G; A/B/C/H; A/B/C/K; A/B/C/L; A/B/C/R; A/C/G/H; A/C/G/K; A/C/G/L; A/C/G/R; A/G/H/K; A/G/H/L; A/G/H/R; A/H/K/L; A/H/K/R; A/K/L/R; B/C/G/H; B/C/G/K; B/C/G/L; B/C/G/R; C/G/H/K; C/G/H/L; C/G/H/R; G/H/K/L; G/H/K/R and H/K/L/R.

The most preferred mixtures of avenanthramides are however A/B, A/C, A/L, B/C, B/L and A/B/C, A/B/L or A/C/L.

In addition, the oat extract can also comprise avenanthramides other than the avenanthramides A, B, C, G, H, K, L (non-Collins abbreviations; CAS number 172549-38-1) (also called 0 or 2pd) and R, which naturally occur in oats, such as avenanthramides D, E, F U, X, Y (also termed 2), AA, CC or 00 or any of the remaining avenanthramide compounds of Table 1 or an avenanthramide analogue compound according to Formula 2 as specified above.

In addition, the oat extract can also further comprise at least one avenanthramide analogue compound according to Formula 2 as specified above, in particular Dihydroavenanthramide D.

The at least one avenanthramide or avenanthramide analogue compound or mixture of avenanthramide or avenanthramide analogue compounds, as described above, may be present in the composition or in the oat extract at a concentration or total amount of 0.0001 to 5.0 wt %, based on the total weight of the composition. In a preferred variant, the composition or oat extract comprises the at least one avenanthramide or avenanthramide analogue compound or mixture of avenanthramides or avenanthramide analogue compounds at a concentration or total amount of 0.0005 to 2.0 wt %, still more preferred at a concentration or total amount of 0.001 to 1.0 wt %, based on the total weight of the composition.

The second main ingredient of the formulation according to the first aspect of the present invention is at least one stabilizer. The at least one stabilizer is selected from the group consisting of chelating agent, organic acid, antioxidants and mixtures thereof.

Within the context of the present invention, the term “at least one stabilizer” is intended to include either one stabilizer only or more than one stabilizer, i.e. two, or even more, i.e. a combination of stabilizers from one stabilizer category or a combination of stabilizer from different stabilizer categories.

A stabilizer within the context of the present invention is a chemical compound that is used to prevent or decrease degradation of an active component, i.e. an avenanthramide, of a composition and, thus extending its shelf life without affecting other components of the composition or a final product in which they are used.

Surprisingly, it turns out that combining with at least one stabilizer selected from the group consisting of chelating agents, organic acids, antioxidants and any mixtures of these is beneficial in improving the stability of an avenanthramide or the stability of compositions comprising an avenanthramide or more avenanthramides. In particular, adding one of the above stabilizers significantly inhibits the degradability of one of the avenanthramides. More surprisingly, it turns out that the addition of one of the stabilizer according to the present invention is particular effective in stabilizing avenanthramide C which is the most unstable avenanthramide compound among the avenanthramides.

Chelating agents are substances that form covalent coordination compounds with metal ions or metal atoms. Their molecules or groups of atoms surround a metallic central ion or atom and, as so-called Lewis, acids, give whole pairs of electrons for binding, and, thus, inactivate traces of metal ions that would otherwise act as catalysts. The chelating agents are also referred to as ligands and the complexes formed as chelates.

Beside the metal chelating effect, chelating agents are also have an antioxidant and preservative boosting effect. Metal ions can react with oxygen to form radicals and other reactive species that can lead to discoloration and odors. In peroxide formulations, metal ions react with the peroxide causing the peroxide to decompose and for radicals to be generated. Additionally, chelating agents have been found to enhance the activity of biocides and so allows significantly lower levels of biocides to be used.

By sequestering undesirable metal ions, chelating agents can prevent precipitation of the salts responsible for water hardness and protect against rancidity or color changes caused by heavy metal. Hence, the binding of the metal atoms stabilizes the smell, structure and color of the finished preparations. This gives compositions or end formulations greated stability and longer shelf life.

In a preferred variant, the chelating agent used in the composition according to the first aspect of the present invention is selected from the group consisting of EDTA or a sodium, calcium or magnesium salt thereof, phytic acid, tetrasodium glutamate diacetate, trisodium ethylendiamine disuccinate, sodium citrate, potassium citrate sodium phytate, sodium gluconate, calcium gluconate, caprylhydroxamic acid, galactaric acid, galacturonic acid, sodium metaphosphate, sodium polyitaconate, disodium etidronate and trisodium methylglycinediacetic acid.

Phytic acid is a six-fold dihydrogenphosphate ester of inositol (specifically, of the myo isomer), also called inositol hexakisphosphate (IP6) or inositol polyphosphate. Phytic acid and phytate have a strong binding affinity to the dietary minerals calcium, iron, and zinc, inhibiting their absorption.

Of the above specified chelating agents, EDTA or a salt thereof, trisodium ethylendiamine disuccinate or tetrasodium glutamate diacetate are particularly preferred, as demonstrated by the following examples, since they have particularly good degradation inhibitory activity. Good degradation inhibitory activity means that even small concentrations of use of these compounds produce the desired inhibition when used according to the invention. EDTA, in particular disodium EDTA, is the most effective single chelating agent, and, thus, preferred at most as stabilizer among the chelating agents.

Additionally, the stabilizer can be an organic carbonic acid having 2 to 10, preferably 3 to 8, carbon atoms and characterized by a carboxyl (—COOH) functional group or gluconolactone. Among the organic carbonic acid, the alpha hydroxy acids (AHA) are preferred.

Alpha hydroxy acids (AHA) are a class of chemical compounds that consist of a carboxylic acid substituted with a hydroxyl group on the adjacent carbon. They may be naturally occurring or synthetic. AHAs are well known for their use in the cosmetic industry. They are often found in products that aid in the reduction of wrinkles, that soften strong, defining lines, and that improve the overall look and feel of the skin. They are also used as chemical peels. Sometimes AHAs are used in cosmetics for other purposes, such as to adjust the pH. Due to their acidity, alpha hydroxy acids allow formulations to have reduced preservative levels which is in particular beneficial for skin associated with dry, sensitive or damaged skin.

The alpha hydroxy acids according to the first aspect of the present invention are selected from the group consisting of gluconic acid, glyceric acid, glycolic acid, isocitric acid, lactic acid, malic acid, citric acid, mandelic acid, azelaic acid, anisic acid, ectoin, ferulic acid, folic acid, levulinic acid, niacin, sebacic acid, salicylic acid, sorbic acid, tartaric acid, 2-hydroxyaotanoic acid, 2-hydroxydecanoic acid, mixtures of these or salts thereof, and gluconolactone.

Moreover, the present invention encompasses also derivatives of the AHAs. Preferably, the AHAs are used in the form of their pharmaceutical salts or mixtures thereof. In particular preferred are the mono- or divalent salts and the ammonium salt. Of the salts, sodium, calcium, magnesium or ammonium are most preferred.

Throughout the present invention, the term “AHAs” includes also the stereo isomers, i.e. the R-enantiomers, the S-enantiomer, or the racemate.

Of the above specified alpha hydroxyl carbonic acids, citric acid, lactic acid, malic acid or tartaric acid are particularly preferred, as demonstrated by the following examples, since they have particularly good degradation inhibitory activity. Good degradation inhibitory activity means that even small concentrations of use of these compounds produce the desired inhibition when used according to the invention. Malic acid or tartaric acid are the most single effective alpha hydroxy acids, and, thus, preferred at most as stabilizer among the alpha hydroxy acids.

Additionally, the stabilizer can be an antioxidant. An antioxidant is a substance that inhibits oxidation or reactions promoted by oxygen, peroxides, or free radicals. The antioxidant according to the first aspect of the present invention is selected from the group consisting of 4-hydroxyacetophenone (p-Hydroxyacetophenone; INCI Hydroxyacetophenone, CAS 99-93-4), ascorbic acid, 6-paradol, uric acid, butylhydroxytoluol (BHT), butylhydroxyanisol (BHA), ascorbyl palmitate, ascorbyl phosphate and salts thereof, carnosine, sodium ascorbate, rutin, tocopherol, tocopheryl acetate, ubiquinone, tropolone and allantoin.

Of the above specified antioxidants, 4-hydroxyacetophenone and ascorbic acid are particularly preferred, as demonstrated by the following examples, since they have particularly good degradation inhibitory activity. Good degradation inhibitory activity means that even small concentrations of use of these compounds produce the desired inhibition when used according to the invention. Ascorbic acid is the most effective single antioxidant, and, thus, preferred at most as stabilizer among the antioxidants.

As demonstrated by the following examples, adding one stabilizer selected from the above described three groups of stabilizers considerably enhances the stability of avenanthramides or compositions comprising one or more avenanthramide(s). In particularly, adding one of the above specified stabilizer considerably inhibits the degradation of avenanthramide C in the composition according to the present invention.

Of the above specified stabilizer, disodium EDTA, ascorbic acid, trisodium ethylenediamine disuccinate, 6-paradol and phytic acid are preferred at most.

Surprisingly, the degradation of the avenanthramides, in particular the degradation of avenanthramide C, can still be further reduced or even inhibited, if the composition according to the present invention comprises one stabilizer from each of two different categories of stabilizer as described before, i.e. one stabilizer from each of chelating agent and alpha hydroxy acid or chelating agent and antioxidant or alpha hydroxyl acid and antioxidant.

In another variant, the degradation of the avenanthramides, in particular avenanthramide C, can still be further reduced or even inhibited, if the composition according to the present invention comprises one stabilizer from each of the three different categories of stabilizer as described before, i.e. one stabilizer from each of the category of stabilizer: chelating agent plus alpha hydroxy acid plus antioxidant. A preferred triple stabilizer combination is disodium EDTA plus ascorbic acid plus citric acid.

A preferred variant of the composition according to the present invention therefore comprises a combination of two or even more stabilizers.

If the stabilizer are used in such combinations, the inhibition of degradation can be increased significantly, compared to the use of one single stabilizer, as it is demonstrated by the following examples.

From the above mentioned combinations, the following combinations of stabilizer compounds are preferred:

Disodium EDTA plus ascorbic acid,
Disodium EDTA plus citric acid;
Disodium EDTA plus 4-hydroxyacetophenone;
Disodium EDTA plus lactic acid;
Disodium EDTA plus maleic acid;
Disodium EDTA plus tartaric acid;
Disodium EDTA plus phytic acid;
Ascorbic acid plus disodium EDTA;
Ascorbic acid plus 4-hydroxyacetophenone;
Ascorbic acid plus tetrasodium glutamate diacetate;
Phytic acid plus tetrasodium glutamate diacetate;
Phytic acid plus ascorbic acid;
Phytic acid plus disodium EDTA;
Phytic acid plus hydroxyacetophenone;
Phytic acid plus malic acid;
4-Hydroxyacetophenone plus disodium EDTA;
4-Hydroxyacetophenone plus ascorbic acid;
4-Hydroxyacetophenone plus phytic acid;
4-Hydroxyacetophenone plus tetrasodium glutamate diacetate;
4-Hydroxyacetophenone plus trisodium ethylenediamine disuccinate;
6-Paradol* plus disodium EDTA;
6-Paradol* plus citric acid;
6-Paradol* plus ascorbic acid;
6-Paradol* plus tetrasodium glutamate diacetate;
Disodium EDTA plus ascorbic acid plus citric acid.

In a preferred embodiment of the present invention, there is even a synergistic effect for the following stabilizer combinations:

Disodium EDTA plus ascorbic acid;
Disodium EDTA plus 4-hydroxyacetophenone;
Disodium EDTA plus lactic acid;
Disodium EDTA plus maleic acid;
Disodium EDTA plus tartaric acid;
Disodium EDTA plus citric acid
Ascorbic acid plus 4-hydroxyacetophenone;
Ascorbic acid plus tetrasodium glutamate diacetate;
Ascorbic acid plus phytic acid;
Citric Acid plus tetrasodium glutamate diacetate
Phytic acid plus tetrasodium glutamate diacetate;
Phytic acid plus ascorbic acid;
Phytic acid plus disodium EDTA;
Phytic acid plus hydroxyacetophenone;
Phytic acid plus malic acid;
4-Hydroxyacetophenone plus disodium EDTA;
4-Hydroxyacetophenone plus ascorbic acid;
4-Hydroxyacetophenone plus phytic acid;
4-Hydroxyacetophenone plus tetrasodium glutamate diacetate;
4-Hydroxyacetophenone plus trisodium ethylenediamine disuccinate.
6-Paradol* plus disodium EDTA;
6-Paradol* plus citric acid;
6-Paradol* plus ascorbic acid;
6-Paradol* plus tetrasodium glutamate diacetate;
Disodium EDTA plus ascorbic acid plus citric acid.
*: 6-Paradol (INCI: hydroxymethoxyhenyl decanone; CAS 27113-22-0) is preferably used as solution in dipropylene glycol (DPG), due to a better solubility in aqueous systems (tradename for such a blend of 6-Paradol in DPG is SymDecanox DPG ex Symrise)

As it is obvious from the following examples, the stabilizers are effective even in low concentrations.

The total amount of the stabilizer present in the composition according to the present invention can be between 0.02 and 0.5 wt %, based on the total weight of the composition. In a preferred variant, the concentration of the total stabilizer in the composition is 0.01 to 0.5 wt %, based on the total weight of the composition, and can even more preferably be between 0.02 and 0.2 wt %, based on the total weight of the composition.

Adding one of the above singular stabilizer or a combination of stabilizers significantly inhibits the degradation of one of the avenanthramides A, B, C or L. The modulation of stabilization for avenanthramide A, B, C or L in sum is in a range of 5% to 94.2%, preferably 10% to 60%, and in particularly modulation of stabilization for avenanthramide C is in a range of preferably 5% to 94.2%, preferably 10% to 6040%, depending on the concentration of the stabilizer as it is demonstrated by the following application examples.

For example, a combination of disodium EDTA and citric acid was more effective when used at 0.1% than was disodium EDTA when used alone at the same dosage, as it leads to a degradation of the avenanthramides by 28.6% as compared to 42.9% for the combination of disodium EDTA and citric acid.

Additionally, adding one of the above singular stabilizer or a combination of stabilizers significantly inhibits the degradation of an avenanthramide analogue compound, particularly Dihydroavenanthramide D.

In addition to the above degradation stability, the at least of one stabilizer in the composition according to the first aspect of the present invention reduces or even inhibits discoloration of the composition during storage, even at low concentrations. This effect is demonstrated by the following application examples. This effect is of particular relevance, since the hitherto existing stabilization with ascorbic acid leads to discoloration in the finished product, in particular in emulsion preparations. It was observed that a composition according to the present invention comprising an oat extract with enriched Avns and a stabilizer is also more stable against light-induced degradation than the Avn-enriched product without a stabilizer. This is particularly observable in the Avn C content.

As demonstrated by the following examples, adding one stabilizer selected from the above described three groups of stabilizers considerably enhances the color stability of avenanthramides or compositions comprising one or more avenanthramide(s). In particularly, adding one of the above stabilizers considerably inhibits the degradation of avenanthramide C in the composition according to the present invention. Enhanced color stability means that even small concentrations of use of these compounds produce the desired inhibition when used according to the invention.

With regard to the stabilizer, reference is made to the above description which is also applicable to the color stability. Of the above specified stabilizer, disodium EDTA, hydroxyacetophenone, tetrasodium glutamate diacetate and 6-paradol are the most preferred single stabilizer regarding the color stability.

Also the color stability of the composition according to the present invention can be further improved, if the composition according to the present invention comprises one stabilizer from each of the three different categories of stabilizer as described before, i.e. one stabilizer from each of the category of stabilizer: chelating agent plus alpha hydroxy acid or chelating agent plus antioxidant or alpha hydroxy acid plus antioxidant, as it is demonstrated by the following examples.

From the above mentioned combinations, the following stabilizers or combinations of stabilizer compounds are preferred with regard to the color stability:

Ascorbic acid;
Citric acid;
Lactic acid;
Malic acid;
Phytic acid;

### 4-Hydroxyacetophenone;

### SymDecanox DPG;

Tetrasodium glutamate diacetate;
Trisodium ethylenediamine disuccinate;
Disodium EDTA plus ascorbic acid;
Disodium EDTA plus citric acid;
Disodium EDTA plus 4-hydroxyacetophenone;
Disodium EDTA plus lactic acid;
Disodium EDTA plus maleic acid;
Disodium EDTA plus tartaric acid;
Disodium EDTA plus phytic acid;
Ascorbic acid plus disodium EDTA;
Ascorbic acid plus 4-hydroxyacetophenone;
Ascorbic acid plus tetrasodium glutamate diacetate;
Ascorbic acid plus trisodium ethylenediamine disuccinate;
Citric acid plus tetrasodium glutamate diacetate;
Phytic acid plus tetrasodium glutamate diacetate;
Phytic acid plus trisodium glutamate diacetate
Phytic acid plus ascorbic acid;
Phytic acid plus disodium EDTA;
4-Hydroxyacetophenone plus disodium EDTA;
4-Hydroxyacetophenone plus ascorbic acid;
4-Hydroxyacetophenone plus trisodium ethylenediamine disuccinate;
6-Paradol* plus disodium EDTA;
6-Paradol* plus disodium EDTA;
6-Paradol* plus citric acid;
Disodium EDTA plus ascorbic acid plus citric acid.

In a preferred embodiment of the present invention, there is even a synergistic effect with regard to color stability, if a chelating agent is used in combination with an alpha hydroxyl acid, or if a chelating agent is used in combination with an antioxidant or if an alpha hydroxy acid is used in combination with an antioxidant. Hence, from the above mentioned combinations, the following combinations of stabilizer compounds are particularly preferred:

Disodium EDTA plus 4-hydroxyacetophenone;
Disodium EDTA plus lactic acid;
Disodium EDTA plus citric acid
Disodium EDTA plus maleic acid;
Disodium EDTA plus tartaric acid;
Ascorbic acid plus disodium EDTA;
Ascorbic acid plus hydroxyacetophenone;
Ascorbic acid plus tetrasodium glutamate diacetate;
Ascorbic acid plus 6-paradol;
Citric acid plus tetrasodium glutamate diacetate;
Phytic acid plus disodium EDTA;
Phytic acid plus trisodium ethylenediamine disuccinate;
Phytic plus tetrasodium glutamate diacetate;
4-Hydroxyacetophenone plus disodium EDTA;
4-Hydroxyacetophenone plus ascorbic acid;
4-Hydroxyacetophenone plus trisodium ethylenediamine disuccinate;
6-Paradox*l plus disodium EDTA;
6-Paradol* plus citric acid;
Disodium EDTA plus ascorbic acid plus citric acid.
*: 6-Paradol (INCI: hydroxymethoxyhenyl decanone; CAS 27113-22-0) is preferably used as solution in dipropylene glycol (DPG), due to a better solubility in aqueous systems (tradename for such a blend of 6-Paradol in DPG is SymDecanox DPG ex Symrise)

As it is obvious from the following examples, the stabilizers can effective stabilize color even in low concentrations.

Particularly preferred mixtures according to the present invention are those in which the composition comprises or consists of:
0.0001 to 5.0 wt % of the at least one avenanthramide or an analogue thereof, or an oat extract comprising the at least one avenanthramide or an analogue thereof; and
0.02 to 0.5 wt % of the at least one stabilizer,
   based on the total weight to the composition.

The composition according to the invention, in particular those characterised as preferred compositions, possess a low avenanthramide degradation and a high color stability. The stability and efficacy of the composition according to the present invention is surprisingly superior to that of compositions comprising one or more avenanthramide(s) only. The cosmetically or pharmaceutically active substances, i.e. avenanthramides, which exhibit biological benefits of great interest, such as anti-inflammatory, antioxidant, anti-itching, anti-irritant and anti-atherogenic activities, are thus longer available in an therapeutic effective amount and thus better reach their intended target.

The composition according to the present invention is also particularly effective and free of any toxicologically or dermatologically critical secondary components; the composition can therefore be used without further concerns in cosmetic or pharmaceutical preparations.

It should be borne in mind that the stabilizers that are used in the composition and in the end preparation are
toxicologically acceptable,
well tolerated by the skin,
stable (in particular in the customary formulations),
preferably odorless and
able to be produced inexpensively (i.e. using standard processes and/or starting from standard precursors)
   in the concentration range relevant to activity and administration.

Hence, the composition according to the present invention is thus beneficial for skin protection and in the prevention and/or treatment of dermatoses.

Another aspect of the present invention therefore relates to the use of the composition according to the first aspect of the present invention as a cosmetic, in particular for use in skin care, scalp care, hair care, nail care or in the prevention and/or treatment of skin conditions, intolerant and sensitive skin, skin irritation, skin reddening, wheals, pruritus (itching), skin aging, wrinkle formation, loss of skin volume, loss of skin elasticity, pigment spots, pigment abnormalities, or dry skin, i.e. for moisturising the skin.

Another aspect of the present invention relates to the composition according to the first aspect of the present invention for use as a medicament.

Due to its aforementioned superior properties, the formulation according to the first aspect of the present invention is particularly useful in the prevention and/or treatment of dermatological or keratological diseases, in particular of dermatological or keratological diseases having a barrier related inflammatory, immunoallergic, atherogenic, xerotic or hyperproliferative component or in the prevention and/or treatment of cardiovascular diseases, allergic reactions, coronary heart disease, for decreasing the level of LDL cholesterol and lipids in blood serum, for reducing blood pressure, for improving sensitivity to insulin and for enabling the control of blood glucose levels.

Due to the particular antioxidative effect of the avenanthramide(s), the present invention also relates to the composition according to the first aspect of the present invention for use in the prevention and/or treatment of dermatological diseases associated with increased ROS production.

Examples of such dermatological disorders include eczema, psoriasis, seborrhoea, dermatitis, erythema, pruritus (itching), otitis, inflammation, irritation, fibrosis, Lichen planus, Pityriasis rosea, Pityriasis versicolor, autoimmune bullous diseases, urticarial, angiodermal and allergic skin reactions, and wound healing, and/or wherein the skin diseases associated with increased ROS production are selected from the group consisting of atopic dermatitis, neurodermitis, psoriasis, rosacea, acneiform eruptions, sebostasis and xerosis.

In a particularly preferred variant of the present invention, the composition comprising or consisting of at least one avenanthramide or an analogue thereof or an oat extract comprising at least one avenanthramide or an analogue thereof according to the present invention is beneficially useful in the prevention and/or treatment of pruritis (itching).

Chronic pruritis is a common symptom associated with various dermatological conditions and systemic diseases, with no known underlying condition in some cases. Chronic pruritis is classified by clinical presentation (for example, association with diseased/inflamed or normal/non-inflamed skin and/or presence of secondary scratch lesions) and underlying causes (of for example dermatological, systemic, neurological, psychosomatic, mixed or undetermined origin).

The use of avenanthramide(s) or an analogue thereof or an oat extract comprising avenanthramide(s) or an analogue thereof for these respective purposes corresponds to a method for imparting the respective therapeutic activity of the active substance by adding a therapeutically effective amount of the active substance or composition.

Within the context of the present invention, an effective amount of a composition is the amount of each active component, i.e. an avenanthramide, that is sufficient to show a benefit, such as a reduction in a symptom associated with the disorder, disease or condition to be treated. When applied to a combination or a composition, as in the present case, the term refers to the amount of the combined active ingredients resulting in the benefit.

Accordingly, the present invention relates to a method for preventing and/or treating dermatological or keratological diseases, in particular dermatological or keratological diseases having a barrier related, inflammatory, immunoallergic, atherogenic, xerotic or hyperproliferative component in a subject in need thereof, wherein the method comprises administering the subject with a therapeutically effective amount of a composition comprising or consisting of: at least one avenanthramide or an analogue thereof, or an oact extract comprising at least one avenanthramide or an analogue thereof; and at least one stabilizer in an amount which is sufficient for the prevention and/or treatment of dermatological or keratological diseases, in particular dermatological or keratological diseases having a barrier related, inflammatory, immunoallergic, atherogenic, xerotic or hyperproliferative component.

Due to its marked effect as described above, the composition according to the first aspect of the present invention is beneficially suitable for the preparation of foods, food supplements, cosmetic, pharmaceutical or veterinary preparations.

The composition according to the present invention can be easily incorporated into conventional foods, food supplements, cosmetic, pharmaceutical or veterinary preparations.

A further aspect of the present invention therefore relates to foods, food supplements, cosmetic, pharmaceutical or veterinary preparations which comprise the composition according to the present invention. In a preferred variant of the present invention, a functional food which includes the composition is provided as an effective ingredient for skin care and/or preventing or ameliorating the above dermatological or keratological disorders.

In a preferred variant, the foods, food supplements, cosmetic, pharmaceutical or veterinary preparations comprise the composition or oat extract according to the present invention or are obtained using the method according to the present invention in an amount of 0.0001 to 10 wt %, more preferred 0.0005 to 5 wt %, most preferred 0.001 to 1 wt %, based on the total weight of the preparation.

Within this context, it is also possible—and in some cases advantageous—to combine the composition according to the present invention with other active compounds, for example other synergistically intensifying substances, such as anti-inflammatories, antibacterial or antimycotic substances, substances having a reddening-alleviating or itch-alleviating action, lenitive substances, moisturisers and/or cooling agents and/or antioxidants, preservatives, (metal) chelating agents, penetration enhancers, and/or cosmetically or pharmaceutically acceptable excipients.

An active substance means a substance or compound that imparts a primary utility to a composition or formulation. Examples of such active substances include antioxidants, preservatives, (metal) chelating agents, penetration enhancers, etc. An excipient refers to an inactive substance used to formulate cosmetics or pharmaceuticals as a result of processing or manufacture.

Since dermatological conditions or diseases are often associated with dry skin, scratched skin, skin lesions or even inflammation, the cosmetic and/or pharmaceutical preparations particularly advantageously contains a skin-moisturising and/or moisture-retaining substance, a cooling agent, an osmolyte, a keratolytic substance, a nurturing substance, an anti-inflammatory, antibacterial or antimycotic substance and/or a substance having a reddening-alleviating or itch-alleviating action and/or a lenitive substance.

Itching occurs with particular intensity when the skin is dry. The use of skin-moisturising and/or moisture-retaining substances or regulators in cosmetic and pharmaceutical preparations can significantly alleviate itching. The cosmetic or pharmaceutical preparations according to the present invention can therefore also be particularly advantageously combined with one or more skin-moisturising and/or moisture-retaining substances or regulators. Cosmetic or pharmaceutical preparations according to the present invention can therefore advantageously also contain the following moisturising and/or moisture-retaining substances or regulators: sodium lactate, urea, urea derivatives, alcohols, glycerol, diols such as propylene glycol, hexylene glycol, 1,2-pentanediol, 1,2-hexanediol, 1,2-heptanediol, 1,2-octanediol, 1,2-nonanediol, 1,2-decanediol or mixtures of said diols, in particular mixtures of 1,2-hexanediol and 1,2-octanediol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulphate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts.

Depending on the substance, the concentration of the moisture retention regulators used is between 0.1 and 10% (m/m) and preferably between 0.5 and 5% (m/m), based on the total weight of a ready-to-use cosmetic or pharmaceutical end product. These data apply in particular to such diols as are advantageously to be used, such as hexylene glycol, 1,2-pentanediol, 1,2-hexanediol, 1,2-octanediol and 1,2-decanediol, as well as mixtures of 1,2-hexanediol and 1,2-octanediol.

The use of cooling agents in cosmetic and pharmaceutical preparations can alleviate itching. The preparations according to the present invention can therefore also be particularly advantageously combined with one or more cooling agent(s). Preferred individual cooling agents for use within the framework of the present invention are listed below. The person skilled in the art can add many other cooling agents to this list; the cooling agents listed can also be used in combination with one another: I-menthol, d-menthol, racemic menthol, menthone glycerol acetal (trade name: Frescolat® MGA), menthyl lactate (trade name: Frescolat® ML; menthyl lactate is preferably I-menthyl lactate, in particular I-menthyl I-lactate), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate, N-acetylglycine menthyl ester, isopulegol, hydroxycarboxylic acid menthyl esters (such as menthyl 3-hydroxybutyrate), monomenthyl succinate, 2-mercaptocyclodecanone, menthyl 2-pyrrolidin-5-one carboxylate, 2,3-dihydroxy-p-menthane, 3,3,5-trimethyl cyclohexanone glycerol ketal, 3-menthyl-3,6-di- and trioxaalkanoates, 3-menthyl methoxyacetate and icilin.

Cooling agents which are preferred due to their particular synergistic effect are I-menthol, d-menthol, racemic menthol, menthone glycerol acetal (trade name: Frescolat® MGA), menthyl lactate (preferably I-menthyl lactate, in particular I-menthyl I-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol.

Particularly preferred cooling agents are I-menthol, racemic menthol, menthone glycerol acetal (trade name: Frescolat® MGA), menthyl lactate (preferably I-menthyl lactate, in particular I-menthyl I-lactate (trade name: Frescolat® ML)), 3-menthoxypropane-1,2-diol, 2-hydroxyethyl menthyl carbonate and 2-hydroxypropyl menthyl carbonate.

Very particularly preferred cooling agents are I-menthol, menthone glycerol acetal (trade name: Frescolat® MGA) and menthyl lactate (preferably I-menthyl lactate, in particular I-menthyl I-lactate (trade name: Frescolat® ML)).

Depending on the substance, the concentration of the cooling agents used is preferably between 0.01 and 20 wt % and particularly preferably between 0.1 and 5 wt %, based on the total weight of a ready-to-use cosmetic or pharmaceutical end product.

The cosmetic or pharmaceutical preparations that contain the composition according to the present invention can also be used together with one or more osmolyte(s). Examples of osmolytes which may be mentioned here include substances from the group comprising sugar alcohols (myoinositol, mannitol, sorbitol), quaternary amines such as taurine, choline, betaine, betaine glycine, ectoin, diglycerol phosphate, phosphorylcholine or glycerophosphorylcholines, amino acids such as glutamine, glycine, alanine, glutamate, aspartate or proline, phosphatidylcholine, phosphatidylinositol, inorganic phosphates, and polymers of said compounds, such as proteins, peptides, polyamino acids and polyols. All osmolytes simultaneously have a skin-moisturising action.

Preferably, keratolytic substances can also be combined with the composition or oat extract according to the present invention. Keratolytic compounds include the large group of alpha-hydroxy acids. Salicylic acid is for example preferably used.

In cosmetic or pharmaceutical preparations that contain the composition according to the present invention for the topical cosmetic or pharmaceutical treatment of for example dry and/or itchy skin, a high proportion of in particular nurturing substances is also particularly advantageous because of the reduced trans-epidermal water loss due to lipophilic components. In one preferred embodiment, the cosmetic or pharmaceutical preparations contain one or more nurturing animal and/or vegetable fats and oils such as olive oil, sunflower oil, refined soybean oil, palm oil, sesame oil, rapeseed oil, almond oil, borage oil, evening primrose oil, coconut oil, shea butter, jojoba oil, sperm oil, tallow, neatsfoot oil and lard, and optionally other nurturing components such as fatty alcohols having 8 to 30 C atoms. The fatty alcohols used here can be either saturated or unsaturated and either linear or branched. Nurturing substances which can be particularly preferably combined with the mixtures according to the present invention also include in particular ceramides, understood here to mean N-acylsphingosines (fatty acid amides of sphingosine) or synthetic analogues of such lipids (so-called pseudo-ceramides) which markedly improve the water retention capacity of the stratum corneum; phospholipids, such as soy lecithin, egg lecithin and cephalins; and petrolatum, paraffin oils and silicone oils, the latter including inter alia dialkyl- and alkylarylsiloxanes such as dimethylpolysiloxane and methylphenylpolysiloxane and their alkoxylated and quaternised derivatives.

Cosmetic or pharmaceutical preparations that contain the composition composition according to the present invention can also contain other anti-inflammatory active compounds or active compounds exhibiting anti-reddening and anti-itching activity. Within this context, any anti-inflammatory active compounds and active compounds that alleviate reddening and itching and are suitable or customary in cosmetic and/or dermatological applications can be used. Advantageously, the anti-inflammatory active compounds and active compounds which alleviate reddening and/or itching that are used are steroidal anti-inflammatory substances of the corticosteroid type, such as for example hydrocortisone, dexamethasone, dexamethasone phosphate, methylprednisolone or cortisone, wherein this list may be expanded by adding other steroidal anti-inflammatory agents. Non-steroidal anti-inflammatory agents can also be used, for example: oxicams, such as piroxicam or tenoxicam; salicylates, such as aspirin, Disalcid®, Solprin® or fendosal; acetic acid derivatives, such as diclofenac, fenclofenac, indomethacin, sulindac, tolmetin or clindanac; fenamates, such as mefenamic, meclofenamic, flufenamic or niflumic acid; propionic acid derivatives, such as ibuprofen, naproxen, benoxaprofen; or pyrazoles, such as phenylbutazone, oxyphenylbutazone, febrazone or azapropazone. Alternatively, natural anti-inflammatory substances and substances that alleviate reddening and/or itching can be used. Plant extracts, special highly active plant extract fractions and also highly pure active substances isolated from plant extracts can be used. Extracts, fractions and active substances from camomile, Aloe vera, Commiphora species, Rubia species, willows, willow-herb, ginger, marigold, Arnica, Glycyrrhiza species, Echinacea species, Rubus species and pure substances such as inter alia bisabolol, apigenin, apigenin-7-glucoside, gingerols such as [6]-gingerol, paradols such as [6]-paradol, boswellic acid, phytosterols, glycyrrhizine, glabridin or licochalcone A are partsincicularly preferred. The preparations containing histamine-release inhibitors can also contain mixtures of two or more anti-inflammatory active compounds.

Cosmetic or pharmaceutical preparations that contain the composition according to the present invention can also contain active compounds for preservative purposes, wherein any preservatives may be used which are suitable or customary in cosmetic and/or dermatological applications and which are advantageously selected from the group consisting of preservatives such as inter alia benzoic acid, its esters and salts; propionic acid and its salts; salicylic acid and its salts; 2,4-hexanoic acid (sorbic acid) and its salts; formaldehyde and paraformaldehyde; 2-hydroxybiphenyl ether and its salts; 2-zincsulphidopyridine N-oxide; inorganic sulphites and bisulphites; sodium iodate; chlorobutanol; 4-hydroxybenzoic acid and its salts and esters; dehydroacetic acid; formic acid; 1,6-bis(4-amidino-2-bromophenoxy)-n-hexane and its salts; the sodium salt of ethylmercury-(II)-thiosalicylic acid; phenylmercury and its salts; 10-undecylenic acid and its salts; 5-amino-1,3-bis(2-ethylhexyl)-5-methylhexahydropyrimidine; 5-bromo-5-nitro-1,3-dioxane; 2-bromo-2-nitro-1,3-propanediol; 2,4-dichlorobenzyl alcohol; N-(4-chlorophenyl)-N′-(3,4-dichlorophenyl)urea; 4-chloro-m-cresol; 2,4,4′-trichloro-2′-hydroxy-diphenyl ether; 4-chloro-3,5-dimethylphenol; 1,1′-methylene-bis(3-(1-hydroxymethyl-2,4-dioximidazolidin-5-yl)urea); poly(hexamethylene biguanide) hydrochloride; 2-phenoxyethanol; hexamethylenetetramine; 1-(3-chloroallyl)-3,5,7-triaza-1-azoniaadamantane chloride; 1-(4-chloro-phenoxy)-1(1H-imidazol-1-yl)-3,3-dimethyl-2-butanone; 1,3-bis(hydroxymethyl)-5,5-dimethyl-2,4-imidazolidinedione; benzyl alcohol; Octopirox®; 1,2-dibromo-2,4-dicyanobutane; 2,2′-methylene-bis(6-bromo-4-chloro-phenol); bromochlorophene; mixture of 5-chloro-2-methyl-3(2H)-isothiazolinone and 2-methyl-3(2H)isothiazolinone with magnesium chloride and magnesium nitrate; 2-benzyl-4-chlorophenol; 2-chloroacetamide; chlorhexidine; chlorhexidine acetate; chlorhexidine gluconate; chlorhexidine hydrochloride; 1-phenoxy-propan-2-ol; N-alkyl(C12-C22)trimethylammonium bromide and chloride; 4,4-dimethyl-1,3-oxazolidine; N-hydroxymethyl-N-(1,3-di(hydroxymethyl)-2,5-dioxoimidazolidin-4-yl)-N′-hydroxymethylurea; 1,6-bis(4-amidinophenoxy)-n-hexane and its salts; glutaraldehyde 5-ethyl-1-aza-3,7-dioxabicyclo(3.3.0)octane; 3-(4-chlorophenoxy)-1,2-propanediol; hyamine; alkyl(C8-C18)dimethylbenzylammonium chloride; alkyl(C8-C18)dimethylbenzylammonium bromide; alkyl(C8-C18)dimethylbenzylammonium saccharinate; benzylhemiformal; 3-iodo-2-propynyl butylcarbamate; or sodium ((hydroxymethyl)amino)acetate.

Other antibacterial or antimycotic active substances can also particularly advantageously be used in the cosmetic or pharmaceutical preparations that contain the composition according to the present invention, wherein any antibacterial or antimycotic active substances can be used which are suitable or customary in cosmetic and/or dermatological applications. In addition to the large group of conventional antibiotics, other products which are advantageous here include those relevant to cosmetics such as in particular triclosan, climbazole, octoxyglycerin, Octopirox® (1-hydroxy-4-methyl-6-(2,4,4-trimethylpentyl)-2(1H)-pyridone 2-aminoethanol salt), chitosan, farnesol, glycerol monolaurate or combinations of said substances, which are used inter alia against underarm odour, foot odour or dandruff.

The cosmetic and/or pharmaceutical preparations according to the present invention can also contain one or more lenitive substances, wherein any lenitive substances can be used which are suitable or customary in cosmetic and/or pharmaceutical applications such as alpha-bisabolol, azulene, guaiazulene, 18-beta-glycyrrhetinic acid, allantoin, Aloe vera juice or gel, extracts of Hamamelis virginiana (witch hazel), Echinacea species, Centella asiatica, chamomile, ginger extracts, gingerols, Arnica monatana, Glycyrrhiza species, algae, seaweed and Calendula officinalis, and vegetable oils such as sweet almond oil, baobab oil, olive oil, panthenol, laureth-9, trideceth-9 and 4-t-butylcyclohexanol.

In addition, the cosmetic or pharmaceutical preparations can also particularly advantageously be used in combination with perspiration-inhibiting active compounds (antiperspirants) for controlling body odour. Perspiration-inhibiting active compounds used include in particular aluminium salts, such as aluminium chloride, chlorohydrate, nitrate, sulphate, acetate, etc. The use of zinc, magnesium or zirconium compounds can however also be advantageous. Aluminium salts and, to a somewhat lesser extent, aluminium/zirconium salt combinations have proven useful in cosmetic and dermatological antiperspirants. Partially neutralised aluminium hydroxychlorides, which are therefore more tolerable to the skin but are not quite as effective, are also noteworthy. Substances other than aluminium salts can also be used, such as for example: (a) protein-precipitating substances such as inter alia formaldehyde, glutaraldehyde, natural and synthetic tanning agents and trichloroacetic acid, which cause surface closure of the sweat glands; (b) local anaesthetics, including dilute solutions of for example lidocaine, prilocaine or mixtures of the same, which switch off the sympathetic supply to the sweat glands by blocking the peripheral nerve paths; (c) zeolites of the X, A or Y type, which reduce sweat secretion and also act as adsorbents for bad odours; and (d) botulinus toxin (the toxin of the bacterium Clostridium botulinum), which is also used in hyperhidrosis (pathological increase in sweat secretion), and the action of which is based on irreversibly blocking the release of the transmitter substance acetylcholine which is relevant to sweat secretion.

A combination with (metal) chelating agents other than the above described chelating agents used according to the invention can also be advantageous in the cosmetic or pharmaceutical preparations that contain the composition according to the present invention, wherein any metal chelating agents can be used which are suitable or customary in cosmetic and/or dermatological applications. Preferred (metal) chelating agents include α-hydroxy fatty acids, phytic acid, lactoferrin, as well as humic acids, bile acids, bile extracts, bilirubin, biliverdin or EGTA and their derivatives.

In order to be used, the preparations containing the composition according to the present invention are applied to the skin, scalp, hair and/or nail in an adequate amount in such manner as is customary with cosmetics and dermatological products. Within this context, cosmetic and dermatological preparations that contain a mixture according to the present invention and which additionally act as a sunscreen offer particular advantages. Advantageously, these preparations contain at least one UVA filter and/or at least one UVB filter and/or at least one inorganic pigment. Within this context, the preparations can take various forms such as are for example customarily employed for this type of preparation, such as for example solutions, water-in-oil (W/O) emulsions, oil-in-water (O/W) emulsions or multiple emulsions such as water-in-oil-in-water (W/O/W) emulsions, gels, hydrodispersions, solid sticks or aerosols.

Preparations that contain the composition according to the present invention can advantageously be combined with substances that absorb UV radiation in the UVB range, the total amount of filter substances being for example 0.01 to 40% (m/m), preferably 0.1 to 10% (m/m), in particular 1.0 to 5.0% (m/m), based on the dry weight of the preparations, in order to provide cosmetic preparations that protect the hair and/or skin against the entire range of ultraviolet radiation. They can also serve as sunscreens for hair. If the preparations according to the present invention contain UVB filter substances, these can be oil-soluble or water-soluble. Advantageous oil-soluble UVB filters include: 3-benzylidene camphor derivatives, preferably 3-(4-methylbenzylidene)camphor, 3-benzylidenecamphor; 4-aminobenzoic acid derivatives, preferably 2-ethylhexyl 4-(dimethylamino)benzoate, amyl 4-(dimethylamino)benzoate; esters of cinnamic acid, preferably 2-ethylhexyl 4-methoxycinnamate, isopentyl 4-methoxycinnamate; esters of salicylic acid, preferably 2-ethylhexyl salicylate, 4-isopropylbenzyl salicylate, homomenthyl salicylate; derivatives of benzophenone, preferably 2-hydroxy-4-methoxybenzophenone, 2-hydroxy-4-methoxy-4′-methylbenzophenone, 2,2′-dihydroxy-4-methoxybenzophenone; esters of benzalmalonic acid, preferably di(2-ethylhexyl) 4-methoxybenzalmalonate, 2,4,6-trianilino-(p-carbo-2′-ethyl-1′-hexyloxy)-1,3,5-triazine. Advantageous water-soluble UVB filters include salts of 2-phenylbenzimidazole-5-sulphonic acid, such as its sodium, potassium or triethanolammonium salts, as well as the sulphonic acid itself; sulphonic acid derivatives of benzophenones, preferably 2-hydroxy-4-methoxybenzophenone-5-sulphonic acid and its salts; sulphonic acid derivatives of 3-benzylidene camphor, such as for example 4-(2-oxo-3-bornylidenemethyl)benzenesulphonic acid, 2-methyl-5-(2-oxo-3-bornylidene-methyl)sulphonic acid and their salts and also 1,4-di(2-oxo-10-sulpho-3-bornylidenemethyl)-benzene and its salts (the corresponding 10-sulphato compounds, such as the corresponding sodium, potassium and triethanolammonium salts), and benzene-1,4-di(2-oxo-3-bornylidenemethyl-10-sulphonic acid.

It can also be advantageous to employ UVA filters, such as are customarily contained in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. The amounts used for the UVB combination can be used analogously.

In cosmetic or pharmaceutical preparations, the composition according to the present invention can advantageously also be combined with other auxiliaries or excipients such as are customarily used in such preparations, such as for example antioxidants, perfume oils, anti-foaming agents, colorants, pigments having a colouring action, thickeners, surface-active substances, emulsifiers, plasticising substances, moistening and/or moisture-retaining substances, fats, oils, waxes or other conventional constituents of a cosmetic preparation, such as alcohols, polyols, polymers, foam stabilisers, electrolytes, organic solvents or silicone derivatives. Any conceivable antioxidants, perfume oils, anti-foaming agents, colorants, pigments having a colouring action, thickeners, surface-active substances, emulsifiers, plasticising substances, moistening and/or moisture-retaining substances, fats, oils, waxes, alcohols, polyols, polymers, foam stabilisers, electrolytes, organic solvents or silicone derivatives that are suitable or customary in cosmetic and/or dermatological applications can be used here in accordance with the invention.

A high content of treatment substances is usually advantageous in preparations containing the composition according to the present invention for the topical prophylactic or cosmetic treatment of the skin. In accordance with a preferred embodiment, the compositions contain one or more animal and/or vegetable treatment fats and oils, such as olive oil, sunflower oil, purified soybean oil, palm oil, sesame oil, rapeseed oil, almond oil, borage oil, evening primrose oil, coconut oil, shea butter, jojoba oil, sperm oil, beef tallow, neatsfoot oil and lard, and optionally other treatment constituents such as for example C8-C30 fatty alcohols. The fatty alcohols used here can be saturated or unsaturated and straight-chain or branched, wherein examples include decanol, decenol, octanol, octenol, dodecanol, dodecenol, octadienol, decadienol, dodecadienol, oleyl alcohol, ricinoleyl alcohol, erucic alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well their guerbet alcohols; this list may be extended as desired to include other alcohols which structurally are chemically related. The fatty alcohols preferably originate from natural fatty acids and are usually prepared from the corresponding esters of the fatty acids by reduction. Fatty alcohol fractions formed by reduction from naturally occurring fats and fat oils can also be used, such as for example beef tallow, peanut oil, colza oil, cottonseed oil, soybean oil, sunflower oil, palm kernel oil, linseed oil, maize oil, castor oil, rapeseed oil, sesame oil, cocoa butter and cocoa fat.

The treatment substances that can preferably be combined with the composition according to the present invention can also include: ceramides, being understood to be N-acylsphingosines (fatty acid amides of sphingosine) or synthetic analogues of such lipids (so-called pseudo-ceramides) which clearly improve the water retention capacity of the stratum corneum; phospholipids, for example soy lecithin, egg lecithin and cephalins; Vaseline, paraffin and silicone oils, the latter including inter alia dialkyl- and alkylaryl-siloxanes such as dimethylpolysiloxane and methylphenylpolysiloxane, as well as their alkoxylated and quaternised derivatives.

Hydrolysed animal and/or vegetable proteins can also advantageously be added to the preparations containing the composition according to the present invention. Advantageous examples in this regard include in particular elastin, collagen, keratin, lactoprotein, soy protein, oat protein, pea protein, almond protein and wheat protein fractions or corresponding hydrolysed proteins, as well as their condensation products with fatty acids, and also quaternised hydrolysed proteins, wherein the use of hydrolysed vegetable proteins is preferred.

If a cosmetic or dermatological preparation containing the composition according to the present invention is a solution or lotion, then solvents which can be used include: water or aqueous solutions; fatty oils, fats, waxes and other natural and synthetic fatty bodies, preferably esters of fatty acids with alcohols having a low C number, such as isopropanol, propylene glycol or glycerol, or esters of fatty alcohols with alkanoic acids having a low C number or with fatty acids; alcohols, diols or polyols having a low C number, and their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products. Mixtures of the abovementioned solvents are in particular used. In the case of alcoholic solvents, water can be an additional constituent.

Cosmetic or pharmaceutical preparations that contain the composition according to the present invention can also contain antioxidants, different from the antioxidants of the composition according to the first aspect of the present invention, wherein any antioxidants suitable or customary in cosmetic and/or dermatological applications can be used. Advantageously, the antioxidants are selected from the group consisting of amino acids (for example glycine, histidine, tyrosine, tryptophan) and their derivatives, imidazoles (for example urocanic acid) and their derivatives, peptides such as D,L-carnosine, D-carnosine, L-carnosine and their derivatives (for example anserine), carotenoids, carotenes (for example α-carotene, β-carotene, lycopene) and their derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and their derivatives (esters, ethers, peptides, lipids, nucleotides, nucleosides and salts) as well as sulphoximine compounds (for example buthionine sulphoximines, homocysteine sulphoximines, buthionine sulphones, penta-, hexa-, hepta-thionine sulphoximine) in very low tolerated doses, and also (metal) chelating agents, for example α-hydroxy fatty acids, palmitic acid, phytic acid, lactoferrin, α-hydroxy acids (for example citric acid, lactic acid, malic acid), humic acid, bile acid, bile extracts, bilirubin, biliverdin, EDTA, EGTA and their derivatives, unsaturated fatty acids and their derivatives (for example γ-linolenic acid, linoleic acid, oleic acid), folic acid and its derivatives, ubiquinone and ubiquinol and their derivatives, Vitamin C and its derivatives (for example ascorbyl palmitate, magnesium ascorbyl phosphate, ascorbyl acetate), tocopherols and their derivatives (for example Vitamin E acetate), Vitamin A and its derivatives (for example Vitamin A palmitate) and also coniferyl benzoate of benzoin resin, rutinic acid and its derivatives, ferrulic acid and its derivatives, butylhydroxytoluene, butylhydroxyanisole, nordihydroguaiacic acid, nordihydroguaiaretic acid, trihydroxybutyrophenone, uric acid and its derivatives, mannose and its derivatives, zinc and its derivatives (for example ZnO, ZnSO₄), selenium and its derivatives (such as selenium methionine), stilbenes and their derivatives (such as stilbene oxide, trans-stilbene oxide), as well as the derivatives (such as salts, esters, ethers, sugars, nucleotides, nucleosides, peptides and lipids) of said active compounds such as are suitable in accordance with the invention.

Cosmetic or pharmaceutical preparations that contain the composition according to the present invention can also contain vitamins and vitamin precursors, wherein any vitamins and vitamin precursors which are suitable or customary in cosmetic and/or dermatological applications can be used. Particular mention may be made here of vitamins and vitamin precursors such as tocopherols, Vitamin A, nicotinic acid and nicotinamide, other B-complex vitamins, in particular biotin, and Vitamin C. Other examples within this group which are preferably used include pantothenyl alcohol and its derivatives, in particular its esters and ethers, as well as derivatives of pantothenyl alcohols obtained cationically, such as for example pantothenyl alcohol triacetate, pantothenyl alcohol monoethyl ether and its monoacetate and also cationic pantothenyl alcohol derivatives.

Cosmetic or pharmaceutical preparations that contain the composition according to the present invention can also contain active compounds having a skin-lightening action, wherein any skin-lightening active compounds that are suitable or customary in cosmetic and/or dermatological applications can be used in accordance with the invention. Advantageous skin-lightening active compounds in this regard include kojic acid, hydroquinone, arbutin, ascorbic acid, magnesium ascorbyl phosphate, resorcinols, liquorice root extracts and their constituents glabridin or licochalcone A, or extracts from Rumex and Ramulus species, extracts from pine species (Pinus) or extracts from Vitis species which contain inter alia skin-lightening stilbene derivatives.

Cosmetic preparations that contain the composition according to the present invention can also contain active compounds having a skin-tanning action, wherein any skin-tanning active compounds that are suitable or customary in cosmetic and/or dermatological applications can be used. Dihydroxyacetone (DHA; 1,3-dihydroxy-2-propanone) may be mentioned here by way of example. DHA can be provided in either monomer or dimer form, the proportion of dimers being predominant in the crystalline form.

Cosmetic or pharmaceutical preparations that contain the composition according to the present invention can also contain mono-, di- and oligo-saccharides such as for example glucose, galactose, fructose, mannose and lactose.

Cosmetic or pharmaceutical preparations that contain the composition according to the present invention can also contain plant extracts, which are usually prepared by extraction of the complete plant, but which in individual cases are also prepared exclusively from the blossom and/or leaves, wood, bark or roots of the plant. With regard to the plant extracts which can be used in accordance with the present invention, reference is made in particular to the extracts listed in the table starting on page 44 of the third edition of Leitfaden zur Inhaltsstoffdeklaration kosmetischer Mittel (Guide to the Declaration of Constituents of Cosmetic Agents), published by Industrieverband Körperpflegemittel und Waschmittel e. V. (IKW) (Industrial Association for Toiletries and Detergents), Frankfurt. Particularly advantageous extracts include aloe, Hamamelis, algae, oak bark, willow-herb, stinging nettles, dead nettles, hops, camomile, milfoil, arnica, calendula, burdock root, horse-tail, hawthorn, linden blossom, cucumber, almonds, pine needles, horse chestnut, sandalwood, juniper, coconut, mango, apricot, orange, lemon, lime, grapefruit, apple, green tea, grapefruit seed, wheat, oats, barley, sage, thyme, basil, rosemary, birch, mallow, bitter-crass, willow bark, restharrow, coltsfoot, althaea, ginseng and ginger root. Of these, particularly preferred extracts include aloe vera, camomile, algae, rosemary, calendula, ginseng, cucumber, sage, stinging nettles, linden blossom, arnica and Hamamelis. Mixtures of two or more plant extracts can also be employed. Extraction agents that can be used for preparing said plant extracts include water, alcohols and mixtures thereof. Preferred alcohols in this context are the lower alcohols such as ethanol and isopropanol, but also polyhydric alcohols such as ethylene glycol, propylene glycol and butylene glycol, specifically both as a sole extracting agent and in mixtures with water. The plant extracts can be used in pure form or dilute form in accordance with the invention.

Cosmetic or pharmaceutical preparations that contain the composition according to the present invention can also contain anionic, cationic, non-ionic and/or amphoteric surfactants, especially if crystalline or microcrystalline solids, for example inorganic micropigments, are to be incorporated into the preparations according to the present invention. Surfactants are amphiphilic substances that are able to dissolve organic, non-polar substances in water. Surfactants are generally classified according to the nature and charge of the hydrophilic part of the molecule. Four groups can be differentiated here: anionic surfactants, cationic surfactants, amphoteric surfactants and non-ionic surfactants.

Anionic surfactants usually contain carboxylate, sulphate or sulphonate groups as functional groups. In aqueous solution, they form negatively charged organic ions in the acid or neutral medium. Cationic surfactants are characterised almost exclusively by the presence of a quaternary ammonium group. In aqueous solution, they form positively charged organic ions in the acid or neutral medium. Amphoteric surfactants contain both anionic and cationic groups and accordingly behave like anionic or cationic surfactants in aqueous solution, depending on the pH value. They have a positive charge in a strongly acid medium and a negative charge in an alkaline medium. In the neutral pH range, by contrast, they are zwitterionic. Polyether chains are typical of non-ionic surfactants. Non-ionic surfactants do not form ions in an aqueous medium.

Anionic surfactants that can advantageously be used include: acyl amino acids (and their salts), such as: acyl glutamates, for example sodium acyl glutamate, di-TEA-palmitoyl aspartate and sodium caprylic/capric glutamate; acyl peptides, for example palmitoyl-hydrolysed lactoprotein, sodium cocoyl-hydrolysed soy protein and sodium/potassium cocoyl-hydrolysed collagen; sarcosinates, for example myristoyl sarcosinate, TEA-lauroyl sarcosinate, sodium lauroyl sarcosinate and sodium cocoyl sarcosinate; taurates, for example sodium lauroyl taurate and sodium methyl cocoyl taurate; acyl lactylates, for example lauroyl lactylate and caproyl lactylate; alaninates; carboxylic acids and derivatives, such as for example lauric acid, aluminium stearate, magnesium alkanolate and zinc undecylenate; ester carboxylic acids, for example calcium stearoyl lactylate, laureth-6 citrate and sodium PEG-4 lauramide carboxylate; ether carboxylic acids, for example sodium laureth-13 carboxylate and sodium PEG-6 cocamide carboxylate; phosphoric acid esters and salts, such as for example DEA-oleth-10 phosphate and dilaureth-4 phosphate; sulphonic acids and salts, such as acyl isethionates, for example sodium/ammonium cocoyl isethionate; alkyl aryl sulphonates; alkyl sulphonates, for example sodium cocomonoglyceride sulphonate, sodium C12-14 olefin sulphonate, sodium lauryl sulphoacetate and magnesium PEG-3 cocamide sulphate; sulphosuccinates, for example dioctyl sodium sulphosuccinate, disodium laureth sulphosuccinate, disodium lauryl sulphosuccinate and disodium undecylenamido MEA-sulphosuccinate; and sulphuric acid esters, such as alkyl ether sulphate, for example sodium, ammonium, magnesium, MIPA, TIPA laureth sulphate, sodium myreth sulphate and sodium C12-13 pareth sulphate, and alkyl sulphates, for example sodium, ammonium and TEA lauryl sulphate.

Cationic surfactants that can advantageously be used include: alkyl amines, alkyl imidazoles, ethoxylated amines and quaternary surfactants.

Quaternary surfactants contain at least one N atom that is covalently bonded to four alkyl or aryl groups. This leads to a positive charge, irrespective of the pH value. Alkyl betaine, alkyl amidopropyl betaine and alkyl amidopropyl hydroxysulphaine are advantageous. The cationic surfactants used can also preferably be chosen from the group of quaternary ammonium compounds, in particular benzyl trialkyl ammonium chlorides or bromides, such as for example benzyl dimethylstearyl ammonium chloride, as well as alkyl trialkyl ammonium salts, for example cetyl trimethyl ammonium chloride or bromide, alkyl dimethyl hydroxyethyl ammonium chlorides or bromides, dialkyl dimethyl ammonium chlorides or bromides, alkyl amide ethyl trimethyl ammonium ether sulphates, alkyl pyridinium salts, for example lauryl or cetyl pyridinium chloride, imidazoline derivatives and compounds of a cationic nature, such as amine oxides, for example alkyl dimethyl amine oxides or alkyl aminoethyl dimethyl amine oxides. Cetyl trimethyl ammonium salts can particularly advantageously be used.

Amphoteric surfactants that can advantageously be used include: acyl/dialkyl ethylene diamine, for example sodium acyl amphoacetate, disodium acyl amphodipropionate, disodium alkyl amphodiacetate, sodium acyl amphohydroxypropyl sulphonate, disodium acyl amphodiacetate and sodium acyl amphopropionate; N-alkyl amino acids, for example aminopropyl alkyl glutamide, alkyl aminopropionic acid, sodium alkyl imidodipropionate and lauroamphocarboxyglycinate.

Non-ionic surfactants that can advantageously be used include: alcohols; alkanolamides, such as cocamides MEA/DEA/MIPA, amine oxides, such as cocoamidopropylamine oxide; esters formed by esterification of carboxylic acids with ethylene oxide, glycerol, sorbitan or other alcohols; ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside; sucrose esters and ethers; polyglycerol esters, diglycerol esters, monoglycerol esters; methyl glucose esters, ester of hydroxy acids.

The use of a combination of anionic and/or amphoteric surfactants with one or more non-ionic surfactants is also advantageous. The surface-active substance can be present in a concentration of between 1 and 98% (m/m) in the preparations containing histamine-release inhibitors in accordance with the invention, based on the dry weight of the preparations.

Cosmetic or pharmaceutical preparations that contain the composition according to the present invention can also be formulated in a form suitable for topical application, for example as lotions, aqueous or aqueous-alcoholic gels, vesicle dispersions or as simple or complex emulsions (O/W, W/O, O/W/O or W/O/W), liquids, semi-liquids or solids, such as milks, creams, gels, cream-gels, pastes or sticks, and can optionally be packaged as an aerosol and take the form of mousses or sprays. These compositions are prepared according to usual methods.

For preparing emulsions, the oil phase can advantageously be chosen from the following group of substances: mineral oils, mineral waxes; fatty oils, fats, waxes and other natural and synthetic fatty bodies, preferably esters of fatty acids with alcohols having a low C number, for example with isopropanol, propylene glycol or glycerol, or esters of fatty alcohols with alkanoic acids having a low C number or with fatty acids; alkyl benzoates; silicone oils such as dimethyl polysiloxanes, diethyl polysiloxanes, diphenyl polysiloxanes and mixed forms thereof. Advantageously, esters of saturated and/or unsaturated, branched and/or straight-chain alkane carboxylic acids having a chain length of 3 to 30 C atoms and saturated and/or unsaturated, branched and/or straight-chain alcohols having a chain length of 3 to 30 C atoms, from the group of esters of aromatic carboxylic acids and saturated and/or unsaturated, branched and/or straight-chain alcohols having a chain length of 3 to 30 C atoms can be used. Preferred ester oils include isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, for example jojoba oil.

The oil phase can also advantageously be chosen from the group comprising branched and straight-chain hydrocarbons and waxes, silicone oils, dialkyl ethers, the group comprising saturated or unsaturated, branched or straight-chain alcohols, and fatty acid triglycerides, specifically triglycerol esters of saturated and/or unsaturated, branched and/or straight-chain alkane carboxylic acids having a chain length of 8 to 24, in particular 12 to 18 C atoms. The fatty acid triglycerides can for example advantageously be chosen from the group comprising synthetic, semi-synthetic and natural oils, for example olive oil, sunflower oil, soybean oil, peanut oil, rapeseed oil, almond oil, palm oil, coconut oil, palm kernel oil and the like. Arbitrary admixtures of such oil and wax components can also advantageously be used. In some cases, it is also advantageous to use waxes, for example cetyl palmitate, as the sole lipid component of the oil phase; the oil phase is advantageously chosen from the group consisting of 2-ethylhexyl isostearate, octyldodecanol, isotridecyl isononanoate, isoeicosane, 2-ethylhexyl cocoate, C12-15 alkyl benzoate, caprylic-capric acid triglyceride and dicaprylyl ether. Mixtures of C12-15 alkyl benzoate and 2-ethylhexyl isostearate, mixtures of C12-15 alkyl benzoate and isotridecyl isononanoate and mixtures of C12-15 alkyl benzoate, 2-ethylhexyl isostearate and isotridecyl isononanoate are particularly advantageous. The hydrocarbons paraffin oil, squalane and squalene can also advantageously be used. The oil phase can advantageously also contain or consist entirely of cyclic or linear silicone oils, although an additional content of other oil phase components in addition to the silicone oil or oils is preferably used. Cyclomethicone (for example, decamethylcyclopentasiloxane) can advantageously be used as the silicone oil. However, other silicone oils can also advantageously be used, such as for example undecamethylcyclotrisiloxane, polydimethylsiloxane and poly(methylphenylsiloxane). Mixtures of cyclomethicone and isotridecyl isononanoate and of cyclomethicone and 2-ethylhexyl isostearate are also particularly advantageous.

The aqueous phase of preparations that contain the composition according to the present invention and are provided in the form of an emulsion can include: alcohols, diols or polyols having a low C number, and their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, as well as alcohols having a low C number, such as ethanol, isopropanol, 1,2-propanediol, glycerol and in particular one or more thickeners, which can advantageously be chosen from the group comprising silicon dioxide, aluminium silicates, polysaccharides and their derivatives, such as hyaluronic acid, xanthan gum, hydroxypropyl methyl cellulose, and particularly advantageously from the group comprising polyacrylates, preferably a polyacrylate from the group comprising so-called carbopols, such as type 980, 981, 1382, 2984, 5984 carbopols, each on their own or in combinations.

The cosmetic or pharmaceutical preparations that contain the composition according to the present invention and are provided in the form of an emulsion advantageously contain one or more emulsifiers commonly used in the art for preparing cosmetic or pharmaceutical preparations.

The cosmetic or pharmaceutical preparation containing the composition according to the present invention may also include a cosmetically or pharmaceutically acceptable carrier, such as (without being limited to) one of the following which are commonly used in the art: lactose, dextrose, sucrose, sorbitol, mannitol, starch, acacia rubber, calcium phosphate, alginate, gelatine, calcium silicate, microcrystalline cellulose, polyvinyl pyrrolidone, cellulose, water, syrup, methyl cellulose, methyl hydroxybenzoate, propyl hydroxybenzoate, talc, magnesium stearate, mineral oil and the like. The cosmetic or pharmaceutical preparations may also include lubricants, wetting agents, sweeteners, flavouring agents, emulsifiers, suspensions, preserving agents and the like, in addition to the above components. Suitable pharmaceutically acceptable carriers and preparations are described in detail in Remington's Pharmaceutical Sciences (19^{th} edition, 1995).

The pharmaceutical preparation may be administered orally or parenterally.

A suitable dosage of the pharmaceutical preparation according to the present invention may be variously prescribed depending on factors such as method of formulation, age, body weight, gender or morbid condition, food, administration time, administration route, excretion rate and reaction sensitivity of the patient.

The pharmaceutical composition according to the present invention may be manufactured in a unit dosage form, by being formulated using the pharmaceutically acceptable carrier and/or excipient according to a method that can be easily executed by an average person skilled in the art to which the present invention pertains.

Finally, the present invention relates to the use of a stabilizer for stabilizing an avenanthramide or a composition comprising an avenanthramide, in particular avenanthramide C, wherein the stabilizer is selected from the group consisting of chelating agents, organic carbonic acids having 2 to 10 carbon atoms, antioxidants and mixtures of these.

While the invention has been specifically shown and described with reference to preferred variants, it will be understood by those skilled in the art that various changes in form and detail may be made to it without departing from the spirit and scope of the invention. Moreover, the invention encompasses any combination of the elements described above, in all possible variations, unless specifically indicated otherwise.

In addition, where features or aspects of the invention are described in terms of Markush groups, those skilled in the art will recognise that the invention is also thereby described in terms of any individual member or sub-group of members of the Markush group.

The present invention shall now be described in detail with reference to the following examples, which are merely illustrative of the present invention, such that the content of the present invention is not limited by or to the following examples.

### EXAMPLES

### Example 1: Stability Test of an Avn-Containing DragoCalm® Extract with Different Stabilizer

The oxidative stability of the glycerol/water-based solution of an DragoCalm® extract as described below with different stabilizer was evaluated against a glycerol/water-based solution of an oat extract without stabilizer.

The stability tests of DragCalm® were preformed with an Oxipres device of Mikrolab or irradiated for 6 hours using a Xenotest 440 instrument (60 W, 1294 kJ/m², wavelength 300-400 nm).

The Oxipres device allows the determination of oxidative resistance (shelf life) of oils. The device is a modification of the bomb method according to ASTM D941, which is based on oxidation with oxygen. The test runs under elevated pressure and temperature, through which the degradation process is accelerated. During the test, the consumption of oxygen results in a pressure drop in the vessel. Decrease of pressure indicates more consumption of oxygen and higher oxidation of the test product. Oils with a high degree of unsaturation are most susceptible to autoxidation. Sunflower oil is an oil with a high degree of unsaturated fatty acids and therefore leads to oxidation.

The device also allows also to simulate storage conditions for substances, which do not have unsaturated fatty acids.

The following test conditions represent a comparable storage of DragoCalm® at 40° C. for one month. Thus, it is an accelerated stability test due to harder conditions such as temperature and pressure.

DragoCalm® is a blend of:

| Product | INCI | Quantity (%) | CAS No. |
|---|---|---|---|
| Glycerol | Glycerin | 48.80 | 56-81-5 |
| Water | Water (Aqua) | 50.00 | 7732-18-5 |
| Potassium | Potassium Sorbate | 0.20 | 24634-61-5 |
| Sorbate | | | |
| Oat Seed | Avena sativa (Oat) Kernel | 1.00 | 84012-26-0 |
| Extract | Extract, comprising | | |
| | Avenanthramide C, | | |
| | Avenanthramide A and | | |
| | Avenanthramide B | | |

Test Procedure:

Stabilizers were presolved in DragoCalm® under stirring at room temperature or, if necessary at a temperature from 40 to 50° C. (i.e. hydroxyacetophenone). The DragoCalm® samples (15 g) were placed into the reaction vessel and were either exposed to 5 bars of oxygen for 24 hours at 70° C. using the Oxipres device.

The evaluation of the samples, i.e. the determination of content of Avns was determined by HPLC (comparison before and after Oxipres treatment).

The content of Avenanthramide C was determined by HPLC before and after Oxipres treatment.

The color stability of the glycerol/water-based solution of the DragoCalm® extract as described before with different stabilizer was evaluated against a glycerol/water-based solution of an oat extract without stabilizer.

Furthermore, the color of the DragoCalm® samples was measured before and after the Oxipres treatment by using the L*a*b* system (Lico 690). The b* value describes the discoloration in the yellowish/red/brownish area, the Δ b* value describes the difference of color before and after Oxipres treatment. The higher the Δ b* value the more discolorated is the test solution.

The following Table 2 includes the results of Avenanthramide C determination and color measurement after Oxipres treatment.

TABLE 2
Stability test of Avenanthramide C in DragoCalm
(before and after Oxipres treatment)

| Concentration | Additives | Start | End | Degradation | |
|---|---|---|---|---|---|
| (%) | (raw material name) | (ppm) | (ppm) | (%) | Δb* |
| DragoCalm | — | 16 | 0 | 100.0 | 8.8 |
| without | | | | | |
| additives | | | | | |
| 0.10 | Disodium EDTA | 16 | 9 | 43.8 | 5.0 |
| 0.05 | Ascorbic acid | 16 | 3 | 81.3 | 8.2 |
| 0.10 | Citric acid | 18 | 1 | 94.4 | 8.2 |
| 0.05 | Citric acid | 26 | 1 | 96.2 | 6.5 |
| 0.10 | Lactic acid | 12 | 1 | 91.7 | 8.4 |
| 0.10 | 4-Hydroxyacetophenone | 18 | 1 | 94.4 | 7.3 |
| 0.10 | Malic acid | 11 | 1 | 90.9 | 8.3 |
| 0.10 | Tartaric acid | 11 | 1 | 90.9 | 9.4 |
| 0.10 | Tetrasodium Glutamate | 13 | 1 | 92.3 | 7.8 |
| | Diacetate | | | | |
| 0.1 | Trisodium | 9 | 3 | 66.7 | 7.0 |
| | Ethylenediamine | | | | |
| | Disuccinate | | | | |
| 0.10 | Phytic Acid | 14 | 2 | 85.7 | 8.2 |
| 1.00 | SymDecanox DPG (1% | 18 | 1 | 94.4 | 9.8 |
| | sol. of 6-Paradol in | | | | |
| | Diproypylene Glycol) | | | | |
| 0.025 + 0.05 | Ascorbic acid + Disodium | 14 | 12 | 14.3 | 4.9 |
| | EDTA | | | | |
| 0.05 + 0.05 | Citric acid + Disodium | 14 | 10 | 28.6 | 2.1 |
| | EDTA | | | | |
| 0.05 + 0.05 | Lactic acid + Disodium | 10 | 7 | 30.0 | 3.1 |
| | EDTA | | | | |
| 0.05 + 0.05 | Malic acid + Disodium | 10 | 7 | 30.0 | 3.4 |
| | EDTA | | | | |
| 0.05 + 0.05 | Tartaric acid + Disodium | 10 | 7 | 30.0 | 3.7 |
| | EDTA | | | | |
| 0.025 + 0.05 | Ascorbic Acid + | 10 | 4 | 60.0 | 3.8 |
| | Tetrasodium Glutamate | | | | |
| | Diacetate | | | | |
| 0.05 + 0.05 | Citric Acid + Tetrasodium | 10 | 2 | 80.0 | 7.5 |
| | Glutamate Diacetate | | | | |
| 0.05 + 0.05 | 4-Hydroxyacetophenone + | 9 | 6 | 33.3 | 3.0 |
| | Disodium EDTA | | | | |
| 0.05 + 0.025 | 4-Hydroxyacetophenone + | 9 | 6 | 33.3 | 5.0 |
| | Ascorbic acid | | | | |
| 0.05 + 0.05 | Phytic acid + Trisodium | 9 | 4 | 55.6 | 6.8 |
| | Ethylenediamine | | | | |
| | Disuccinate | | | | |
| 0.05 + 0.05 | Phytic Acid + Tetrasodium | 19 | 7 | 63.2 | 8.3 |
| | Glutamate Diacetate | | | | |
| 0.05 + 0.05 | Phytic Acid + Ascorbic | 20 | 8 | 60.0 | 13.4 |
| | Acid | | | | |
| 0.05 + 0.05 | Phytic Acid + Disodium | 20 | 12 | 40.0 | 5.9 |
| | EDTA | | | | |
| 0.05 + 0.05 | Phytic Acid + 4- | 20 | 3 | 85.0 | 9.1 |
| | Hydroxyacetophenone | | | | |
| 0.05 + 0.05 | Phytic Acid + Malic Acid | 18 | 3 | 83.3 | 10.7 |
| 0.05 + 0.05 | 4-Hydroxyacetophenone + | 19 | 3 | 84.2 | 9.3 |
| | Tetrasodium Glutamate | | | | |
| | Diacetate | | | | |
| 0.05 + 0.05 | 4-Hydroxyacetophenone + | 19 | 9 | 52.6 | 7.7 |
| | Trisodim Ethylendiamine | | | | |
| | Disuccinate | | | | |
| 0.5 + 0.05 | SymDecanox DPG* + | 18 | 12 | 33.3 | 5.3 |
| | Disodium EDTA | | | | |
| 0.5 + 0.05 | SymDecanox* + Citric | 17 | 1 | 94.1 | 7.1 |
| | Acid | | | | |
| 0.5 + 0.025 | SymDecanox* + Ascorbic | 35 | 4 | 88.6 | 12.9 |
| | Acid | | | | |
| 0.5 + 0.05 | SymDecanox* + | 35 | 5 | 85.7 | 11.9 |
| | Tetrasodium Glutamate | | | | |
| | Diacetate | | | | |
| 0.05 + 0.025 + | Disodium ETDA + | 27 | 24 | 11.1 | 4.4 |
| 0.025 | Ascorbic acid + | | | | |
| | Citric acid | | | | |

*SymDecanox DPG is a blend of 6-Paradol in dipropylene glycol

It could be clearly observed that the addition of combinations of acids, chelators and antioxidants leads to an improved stability of Avenanthramide C. Furthermore, it could also be shown that the tested samples showed less discoloration.

Also for Avenanthramide L an improved stability could be observed by the addition of stabilizers.

The test procedure and the evaluation of the samples were carried out in the same manner as described above for Avenanthramide C.

TABLE 3
Stability test of Avenanthramide L in DragoCalm
(before and after Oxipres treatment)

| | Additives | Degradation of |
|---|---|---|
| Concentration | (raw material | Avenanthramide |
| (%) | name) | L (%) |
| DragoCalm | — | |
| without additives | | 11.1 |
| 0.10 | Disodium EDTA | 0.0 |
| 0.05 | Ascorbic acid | 11.1 |
| 0.1 | Citric acid | 22.2 |
| 0.1 | 4-Hydroxyacetophenone | 11.1 |
| 0.1 | Allantoin | 11.1 |
| 0.2 | Trisodium MGDA | 20.0 |
| 0.10 | Phytic acid | 20.0 |
| 1.0 | SymDecanox DPG* | 14.3 |
| 0.1 | Trisodium Ethylenediamine | 0 |
| | Disuccinate | |
| 0.1 | Lactic Acid | 11.1 |
| 0.05 + 0.10 | Ascorbic acid | 0.0 |
| | Disodium EDTA | |
| 0.025 + 0.05 | Ascorbic acid | 0.0 |
| | Disodium EDTA | |
| 0.05 + 0.05 | Citric acid | 0.0 |
| | Disodium EDTA | |
| 0.1 | Malic Acid | 0.0 |
| 0.05 + 0.05 | Lactic acid | 0.0 |
| | Disodium EDTA | |
| 0.05 + 0.05 | Malic acid | 0.0 |
| | Disodium EDTA | |
| 0.05 + 0.05 | 4-Hydroxyacetophenone | 0.0 |
| | Disodium EDTA | |
| 0.05 + 0.025 | 4-Hydroxyacetophenone | 0.0 |
| | Ascorbic acid | |
| 0.05 + 0.05 | Phytic acid | 0.0 |
| | Trisodium Ethylenediamine | |
| | Disuccinate | |
| 0.5 + 0.05 | SymDecanox DPG* | 0.0 |
| | Disodium EDTA | |
| 0.05 + 0.05 | SymDecanox DPG* | 0.0 |
| | Citric acid | |
| 0.5 + 0.025 | SymDecanox DPG* | 10.0 |
| | Ascorbic acid | |
| 0.025 + 0.05 | Ascorbic acid | 0.0 |
| | Citric acid | |

*SymDecanox DPG is a blend of 6-Paradol in dipropylene glycol

The above results clearly show, that the addition of a stabilizer leads to an improved stability of Avenanthramide L and the stability of Avenanthramide L can even synergistically improved by the addition of a combination of the following stabilizers:

4-Hydroxyacetophenone plus ascorbic acid
Symdecanox DPG plus citric acid
Symdecanox DPG plus ascorbic acid

Also for Avenanthramide B an improved stability could be observed by the addition of stabilizers.

The test procedure and the evaluation of the samples were carried out in the same manner as described above for Avenanthramide C.

TABLE 4
Stability test of Avenanthramide B in DragoCalm
(before and after Oxipres treatment)

| | Additives | Degradation of |
|---|---|---|
| Concentration | (raw material | Avenanthramide |
| (%) | name) | B (%) |
| Dragocalm without | — | 14.8 |
| additives | | |
| 0.1 | Disodium EDTA | 3..8 |
| 0.05 | Ascorbic acid | 11.5 |
| 0.1 | Citric acid | 19.2 |
| 0.1 | Tartaric acid | 19.2 |
| 0.1 | 4-Hydroxyacetophenone | 18.5 |
| 0.1 | Phytic acid | 14.8 |
| 0.05 + 0.10 | Ascorbic acid | 0 |
| | Disodium EDTA | |
| 0.025 + 0.05 | Ascorbic acid | 3.7 |
| | Disodium EDTA | |
| 0.05 + 0.05 | Citric acid | 3.7 |
| | Disodium EDTA | |
| 0.1 | Tetrasodium Glutamate | 14.8 |
| | Diacetate | |
| 1.0 | SymDecanox DPG | 20.0 |
| 0.05 + 0.05 | Malic acid | 3.7 |
| | Disodium EDTA | |
| 0.05 | Malic acid | 3.7 |
| 0.05 + 0.05 | Tartaric acid | 0 |
| | Disodium EDTA | |
| 0.025 + 0.05 | Ascorbic Acid | 3.8 |
| | Tetrasodium Glutamate | |
| | Diacetate | |
| 0.05 + 0.05 | Citric Acid | 11.1 |
| | Tetrasodium Glutamate | |
| | Diacetate | |
| 0.10 | Trisodium Ethylenediamine | 7.4 |
| | Disuccinate | |
| 0.05 + 0.05 | 4-Hydroxyacetophenone | 3.7 |
| | Disodium EDTA | |
| 0.05 + 0.025 | 4-Hydroxyacetophenone | 3.7 |
| | Ascorbic acid | |
| 0.05 + 0.025 | Trisodium Ethylenediamine | 11.1 |
| | Disuccinate | |
| | Ascorbic acid | |
| 0.05 + 0.05 | Phytic acid | 7.4 |
| | Trisodium Ethylenediamine | |
| | Disuccinate | |
| 0.05 + 0.05 | Phytic Acid | 7.9 |
| | Tetrasodium Glutamate | |
| | Diacetate | |
| 0.05 + 0.05 | Phytic Acid | 7.9 |
| | Ascorbic Acid | |
| 0.05 + 0.05 | Phytic Acid | 2.6 |
| | Disodium EDTA | |
| 0.05 + 0.05 | Phytic Acid | 13.2 |
| | 4-Hydroxyacetophenone | |
| 0.05 + 0.05 | 4-Hydroxyacetophenone | 10.5 |
| | Tetrasodium Glutamate | |
| | Diacetate | |
| 0.05 + 0.025 + 0.05 | Disodium EDTA | 3.3 |
| | Ascorbic acid | |
| | Citric acid | |
| 0.50 + 0.05 | SymDecanox DPG* | 3.4 |
| | Dinatrium EDTA | |

*SymDecanox DPG is a blend of 6-Paradol in dipropylene glycol

The above results clearly show, that the addition of a stabilizer leads to an improved stability of Avenanthramide B and the stability of Avenanthramide B can even synergistically improved by the addition of a combination of the following stabilizers:

Ascorbic acid plus tetrasodium glutamate diacetate;
Citric acid plus tetrasodium glutamate diacetate;
4-Hydroxyacetophenone plus ascorbic acid;
Phytic acid plus tetrasodium glutamate diacetate;
Phytic acid plus ascorbic acid;
Phytic acid plus 4-hydroxyacetophenone;
Tetrasodium glutamate diacetate plus 4-hydroxyacetophenone;
Disodium EDTA plus ascorbic acid plus citric acid.

### Example 2: Stability Test of Dihydroavenanthramide D (Before and After UV Stress Treatment)

Dihydroavenanthramide D is more stable in comparison to Avenanthramide C, but it is known that degradation occurs after treatment by UV stress.

To evaluate the influence of different stabilizers on the stability of Dihydroavenanthramide D, the following solutions (solutions A to D, solution A serves for comparison) were prepared and treated by a UV stress test for 6 h.

Before and after UV stress treatment the content of Dihydroavenanthramide D was measured by HPLC and additionally the Δ b* value was calculated. The Δ b* value gives information about the grade of yellow/brownish color of a solution. Higher Δ b* values indicate more discoloration.

TABLE 5
Test solution of Dihydroavenantram ide D (w/w %)

| | | Solution A |
|---|---|---|
| | | without |
| Ingredient | INCI | stabilizer |
| Dihydroavenanthramide D | Hydroxyphenyl | 0.05 |
| | Propamidobenzioc Acid | |
| Dipropylene glycol | Dipropylene Glycol | 15.00 |
| Water | aqua | ad 100 |

Dihydroavenanthramide D was presolved in dipropylene glycol by heating the solution up to 50° C. Water was added and stirred until a homogenous solution was achieved.

Test Conditions UV Stress Test:

Xenotest 440 device
6 hours
1294 kJ/m²

### Wavelength 300-400 nm

TABLE 6
Stability test of Dihydroavenanthramide D (before and after UV stress treatment)

| | | | | | Degradation | |
|---|---|---|---|---|---|---|
| | Concentration | | | DHAvD | of DHAvD | |
| Solution | (w/w %) | | UV | (ppm) | (%) | Δb* |
| A | 0.05 | Dihydroavenanthramide | start | 446 | 4.7 | |
| without | | D | end | 425 | | 2.2 |
| stabilizer | | (DHAvD) | | | | |
| B | 0.05 | Dihydroavenanthramide | start | 578 | 0.5 | |
| | 0.025 | D | end | 575 | | 0.2 |
| | 0.05 | Ascorbic acid | | | | |
| | | Citric acid | | | | |
| C | 0.05 | Dihydroavenanthramide | start | 461 | 1.1 | |
| | 0.50 | D | end | 456 | | 1.4 |
| | 0.05 | SymDecanox DPG* | | | | |
| | | Citric acid | | | | |
| D | 0.05 | Dihydroavenanthramide | start | 546 | 0.0 | |
| | 0.50 | D SymDecanox DPG* | end | 548 | | 4.4 |
| | 0.05 | Tetrasodium glutamate | | | | |
| | | diacetate | | | | |

*Symdecanox DPG is a blend of 6-Paradol in dipropylene glycol

It could be Observed that the Following Combinations Lead to Improved Stability of Dihydroavenanthramide D:

Ascorbic acid plus citric acid
SymDecanox DPG plus citric acid
SymDecanox DPG plus tetrasodium glutamate diacetate

The results in Tables 2 to 4 and 6 clearly show that the oat extract stabilized with at least one stabilizer is more stable against degradation and discoloration than oat extract without stabilisator. Furthermore, the results clearly show that some of the stabilizer combinations have even a synergistic effect on degradation of avenanthramides and color stability of the oat extract and of avenanthramide analogue compounds, in particular Dihydroavenanthramide D.

### Example 3: Formulation Examples

TABLE 7
Perfume oil 1 (PO1) (amounts in ‰ by weight)

| Ingredients | Amount |
|---|---|
| ALDEHYDE C14 SO-CALLED | 2 |
| ALLYL AMYL GLYCOLATE 10% DPG | 5 |
| ANISIC ALDEHYDE PURE | 5 |
| APPLE OLIFFAC TYPE | 10 |
| Benzylacetat | 50 |
| BERGAMOT IDENTOIL ® COLOURLESS | 15 |
| CANTHOXAL | 5 |
| CETALOX 10% IPM | 3 |
| CITRONELLOL 950 | 40 |
| DAMASCENONE TOTAL 1% DPG | 5 |
| DAMASCONE ALPHA 10% DPG | 5 |
| DAMASCONE DELTA 10% DPG | 2 |
| DIMETHYL BENZYL CARBINYL BUTYRATE | 2 |
| DIPROPYLENE GLYCOL | 178 |
| EBANOL | 2 |
| ETHYL DECADIENOATE TRANS CIS-2.4 | 2 |
| 10% IPM | |
| FLOROSA | 5 |
| FRAMBINON ® 10% DPG | 7 |
| GALAXOLIDE 50% IN IPM | 100 |
| GALBEX TYPE BASE | 1 |
| GERANYL ACETATE PURE | 2 |
| HEDIONE | 30 |
| HELIOTROPIN | 10 |
| HEXENYL ACETATE CIS-3 10% DPG | 1 |
| HEXENYL SALICYLATE CIS-3 | 5 |
| HEXYL CINNAMIC ALDEHYDE ALPHA | 70 |
| HEXYL SALICYLATE | 50 |
| HYDROXY CITRONELLAL | 10 |
| ISO E SUPER | 15 |
| ISORALDEINE 70 | 20 |
| LEAFOVERT ® | 1 |
| LILIAL | 60 |
| LINALOOL | 60 |
| LINALYL ACETATE | 20 |
| LYRAL | 7 |
| MANZANATE | 2 |
| PHENOXANOL | 7 |
| PHENYLETHYL ALCOHOL | 120 |
| SANDAL MYSORE CORE | 2 |
| SANDRANOL ® | 7 |
| STYRALYL ACETATE | 3 |
| TAGETES RCO 10% TEC | 2 |
| TERPINEOL PURE | 20 |
| TETRAHYDROGERANIOL 10% DPG | 5 |
| TONALIDE | 7 |
| VERTOCITRAL 10% DPG | 5 |
| VERTOFIX | 15 |
| Total | 1000 |

TABLE 8
Perfume oil 2 (PO2) (amounts in ‰ by weight)

| Ingredients | Amount |
|---|---|
| Acetophenone, 10% in DPG | 10 |
| n-Undecanal | 5 |
| Aldehyde C14. so-called (peach aldehyde) | 15 |
| Allylamyl glycolate, 10% in DPG | 20 |
| Amyl salicylate | 25 |
| Benzyl acetate | 60 |
| Citronellol | 80 |
| d-Limonene | 50 |
| Decenol trans-9 | 15 |
| Dihydromyrcenol | 50 |
| Dimethylbenzylcarbinyl acetate | 30 |
| Diphenyloxide | 5 |
| Eucalyptol | 10 |
| Geraniol | 40 |
| Nerol | 20 |
| Geranium oil | 15 |
| Hexenol cis-3, 10% in DPG | 5 |
| Hexenyl salicylate cis-3 | 20 |
| Indole, 10% in DPG | 10 |
| Alpha-ionone | 15 |
| Beta-ionone | 5 |
| Lilial ® (2-methyl-3-(4-tert-butyl- | 60 |
| phenyl)propanal) | |
| Linalool | 40 |
| Methylphenyl acetate | 10 |
| Phenylethyl alcohol | 275 |
| Styrolyl acetate | 20 |
| Terpineol | 30 |
| Tetrahydrolinalool | 50 |
| Cinnamyl alcohol | 10 |
| Total: | 1000 |

TABLE 9
Perfume oil 3 (PO3) (amounts in ‰ by weight)

| Ingredients | Amount |
|---|---|
| Benzyl acetate | 60 |
| Citronellyl acetate | 60 |
| Cyclamenaldehyde (2-methyl-3-(4- | 20 |
| isopropylphenyl)propanal | |
| Dipropylene glycol (DPG) | 60 |
| Ethyllinalool | 40 |
| Florol (2-isobutyl-4-methyltetrahydro-2H-pyran-4-ol) | 30 |
| Globanone ® [(E/Z)-8-cyclohexadecen-1-one] | 180 |
| Hedione ® (methyldihydrojasmonate) | 140 |
| Hexenyl salicylate, cis-3 | 10 |
| Vertocitral (2.4-dimethyl-3- | 5 |
| cyclohexenecarboxaldehyde) | |
| Hydratropaldehyde, 10% in DPG | 5 |
| Isodamascone (1-(2.4.4-trimethyl-2-cyclohexen-1-yl)- | 5 |
| 2-buten-1-one, 10% in DPG | |
| Isomuscone (cyclohexadecanone) | 40 |
| Jacinthaflor (2-methyl-4-phenyl-1.3-dioxolane) | 10 |
| Cis-jasmone, 10% in DPG | 20 |
| Linalool | 50 |
| Linalyl acetate | 30 |
| Methyl benzoate, 10% in DPG | 25 |
| para-Methyl cresol, 10% in DPG | 10 |
| Nerol | 20 |
| Phenylpropylaldehyde | 5 |
| 2-Phenylethyl alcohol | 82 |
| Tetrahydrogeraniol | 13 |
| 2.2-Dimethyl-3-cyclohexyl-1-propanol | 80 |
| Total: | 1000 |

TABLE 10
Perfume oil 4 (PO4) (amounts in ‰ by weight)

| Ingredients | Amount |
|---|---|
| AMBRETTOLIDE (MACRO) | 10 |
| AMBROXIDE 10% in IPM | 10 |
| BENZYL ACETATE | 20 |
| BENZYL SALICYLATE | 15 |
| BERGAMOT OIL. bergapten-free | 60 |
| CALONE ® 1951 10% in DPG | 15 |
| COUMARIN | 5 |
| CYCLOGALBANATE ® 10% in DPG | 10 |
| ALPHA -DAMASCONE 1% in DPG | 20 |
| DIHYDROMYRCENOL | 10 |
| ETHYL LINALOOL | 75 |
| ETHYL LINALYLACETATE | 50 |
| ETHYL MALTOL 1% in DEP | 10 |
| ETHYLENE BRASSYLATE (MACRO) | 80 |
| FLOROSA | 40 |
| GERANYLACETATE | 10 |
| HEDIONE ® HC/30 | 35 |
| HEDIONE ® | 210 |
| HELIONAL ® | 15 |
| HELVETOLIDE ® (ALICYC) | 30 |
| HEXENYLSALICYLATE CIS-3 | 20 |
| ISO E SUPER ® | 40 |
| LEAFOVERT ®, 10% in DEP | 10 |
| LILIAL ® | 80 |
| LYRAL ® | 20 |
| MANDARIN OIL | 10 |
| STYRALYL ACETATE | 5 |
| SYMROSE ® | 15 |
| VANILLIN, 10% in DEP | 20 |
| DIPROPYLENE GLYCOL (DPG) | 50 |
| TOTAL | 1000 |

TABLE 11
Perfume oil 5 (PO5) (amounts in ‰ by weight)

| Ingredients | Amount |
|---|---|
| AMAROCITE ® | 10 |
| AMBROCENIDE ® 10% in DPG | 5 |
| AMBROXIDE | 15 |
| AURELIONE ® (7/8-Cyclohexadecenone) (MACRO) | 70 |
| BERGAMOT OIL. bergapten-free | 90 |
| CALONE ® 1951 10% in DPG | 20 |
| CARAWAY OIL | 10 |
| CITRAL | 20 |
| COUMARIN | 10 |
| ALPHA-DAMASCONE, 1% in DPG | 15 |
| DIHYDROMYRCENOL | 70 |
| ESTRAGON OIL | 10 |
| ETHYL LINALOOL | 100 |
| ETHYL LINALYLACETATE | 90 |
| EUGENOL | 10 |
| EVERNYL ® | 5 |
| FRUCTATE ® | 5 |
| GERANIUM OIL | 5 |
| HEDIONE ® HC/30 | 100 |
| HELIONAL ® | 10 |
| INDOLE, 10% in DPG | 5 |
| ISO E SUPER ® | 100 |
| KEPHALIS ® | 5 |
| LAVENDER OIL | 40 |
| CITRUS OIL | 80 |
| LILIAL ® | 30 |
| MANDARIN OIL | 20 |
| MUSCENONE (MACRO) | 5 |
| SANDRANOL ® | 10 |
| VANILLIN 10% in DPG | 5 |
| DIPROPYLENE GLYCOL | 30 |
| TOTAL | 1000 |

The above perfume oils PO1, PO2, PO3, PO4, or PO5 were worked separately in each case into the preparations/formulations presented below.

Cosmetic preparations/formulations (amounts in % by weight for all preparations/formulations)

TABLE 12
Cream, o/w

| Ingredients | INCI | Amount |
|---|---|---|
| Dracorin ® CE | Glyceryl Stearate Citrate | 1.0 |
| Lanette ® O | Cetearyl Alcohol | 2.0 |
| Cutina ® GMS-V | Glyceryl Stearate | 1.0 |
| Tegosoft ® MM | Myristyl Myristate | 1.0 |
| Xiameter ® PMX-0246 | Cyclohexasiloxane, | 0.5 |
| | Cyclopentasiloxane | |
| Dragoxat ® 89 | Ethylhexyl Isononanoate | 2.0 |
| PCL-Liquid 100 | Cetearyl Ethylhexanoate | 4.0 |
| Neutral Oil | Caprylic/Capric Triglyceride | 4.0 |
| Carbopol ® Ultrez 21 | Acrylates/C10-30 Alkyl Acrylate | 0.2 |
| | Crosspolymer | |
| Keltrol ® CG-T | Xanthan Gum | 0.1 |
| Water | Water (Aqua) | ad 100 |
| Glycerol 99.5 P. | Glycerol | 3.0 |
| Hydrolite CG | Caprylyl Glycol | 0.2 |
| 1.2-Propylene Glycol 99 | Propylene Glycol | 2.0 |
| P GC | | |
| Sodium Benzoate | Sodium Benzoate | 0.1 |
| Sodium Hydroxide | Sodium Hydroxide | 0.5 |
| (10% solution) | | |
| Perfume oil PO1, PO2, | Perfume | 0.3 |
| PO3, PO4, or PO5 | | |
| Euxyl ® K702 | Dehydroacetic Acid, Benzoic | 0.3 |
| | Acid, | |
| | Phenoxyethanol, Polyaminopropyl | |
| | Biguanide, Ethylhexylglycerin | |
| Avenanthramide A, B | Pentylene Glycol, Butylene | 1.0 |
| and C ≥ 100 | Glycol, Hydroxyphenyl | |
| ppm in sum in | Propamidobenzoic Acid | |
| glycerine/water | | |
| EDTA BD | Disodium EDTA | 0.05 |
| Vitamin C | Ascorbic Acid | 0.025 |

TABLE 13
Hand and body cream

| Ingredients | INCI | Amount |
|---|---|---|
| Dracorin ® GOC | Glyceryl Oleate Citrate, | 2.0 |
| | Caprylic/Capric Triglyceride | |
| PCL-Solid | Stearyl Heptanoate, Stearyl | 2.5 |
| | Caprylate | |
| Lanette ® O | Cetearyl Alcohol | 1.5 |
| Cutina ® GMS-V | Glyceryl Stearate | 1.0 |
| Dragoxat ® 89 | Ethylhexyl Isononanoate | 3.0 |
| PCL-Liquid 100 | Cetearyl Ethylhexanoate | 7.0 |
| Isodragol ® | Triisononanoin | 4.0 |
| Xiameter ® PMX-0345 | Cyclopentasiloxane (and) | 0.5 |
| Cyclosiloxane | Cyclohexasiloxane | |
| Water | Water (Aqua) | ad 100 |
| Carbopol ® Ultrez 21 | Acrylates/C10-30 Alkyl Acrylate | 0.2 |
| | Crosspolymer | |
| Keltrol ® CG-RD | Xanthan Gum | 0.1 |
| Glycerol 85 P, | Glycerol | 3.0 |
| DragoBetaGlucan | Water (Aqua), Butylene Glycol, | 1.5 |
| | Glycerol, Avena Sativa (Oat) | |
| | Kernel Extract | |
| Potassium Sorbate | Potassium Sorbate | 0.3 |
| Hydrolite-6 | 1,2 Hexanediol | 1.0 |
| Sodium Hydroxide | Sodium Hydroxide | 0.5 |
| (10% solution) | | |
| Perfume oil PO1, PO2, | Fragrance | 0.2 |
| PO3, PO4, or PO5 | | |
| Avenanthramide L | Avenanthramide L | 0.1 |
| Citric Acid | Citric Acid | 0.025 |
| EDTA BD | Disodium EDTA | 0.05 |

TABLE 14
Daily face cream SPF 20

| Ingredients | Amount |
|---|---|
| SymOcide PH | 1 |
| Phenoxyethanol, 4-hydroxyacetophenone, Caprylyl | |
| Glycol, Water (Aqua) | |
| Ascorbyl Palmitate | 0.1 |
| Ascorbyl Palmitate | |
| Biotive L-Arginine | 0.2 |
| Arginine | |
| Buriti oil | 1 |
| Mauritia Flexuosa Fruit Oil | |
| Cocoa butter | 2 |
| Theobroma Cacao (Cocoa) Seed Butter | |
| Dimethicone | 0.5 |
| Dimethicone | |
| Disodium EDTA | 0.1 |
| Disodium EDTA | |
| Dragosantol 100 | 0.1 |
| Bisabolol | |
| Dragoxat 89 | 5 |
| Ethylhexyl Isononanoate | |
| Emulsiphos | 2 |
| Potassium Cetyl Phosphate, Hydrogenated Palm | |
| Glycerides | |
| Extrapone Corail | 1 |
| Glycerin, Aqua, Hydrolyzed Corallina Officinalis | |
| Glycerin | 3 |
| Glycerin | |
| Isoadipate | 5 |
| Diisopropyl Adipate | |
| Jojoba Wax Flakes | 1 |
| Hydrogenated Jojoba Oil | |
| Keltrol CG-T | 0.1 |
| Xanthan Gum | |
| Lanette O | 5 |
| Cetearyl Alcohol | |
| Lanette 16 | 1 |
| Cetyl Alcohol | |
| Lanette 22 | 1 |
| Behenyl Alcohol | |
| Neo Heliopan 357 | 3 |
| Butyl Methoxydibenzoylmethane | |
| Neo Heliopan HMS | 10 |
| Homosalate | |
| Neo Heliopan Hydro used as a 25% aq, Solution | 8 |
| neutralized by arginine | |
| Phenylbenzimidazole Sulfonic Acid | |
| Neo Heliopan OS | 5 |
| Ethylhexyl Salicylate | |
| Orgasol Caresse | 1 |
| Polyamide-5 | |
| Perfume oil PO1, PO2, PO3, PO4, or PO5 | 0.1 |
| Shea butter | 3 |
| Butyrospermum Parkii (Shea) Butter | |
| Simugel EG | 1 |
| Sodium Acrylate/Sodium Acryloyldimethyl Taurate | |
| Copolymer. Isohexadecane. Polysorbate 80 | |
| SymFinity 1298 | 0.1 |
| Echinacea Purpurea Extract | |
| SymDiol 68 | 0.5 |
| 1,2 Hexanediol, Caprylyl Glycol | |
| SymMatrix | 0.1 |
| Maltodextrin, Rubus Fructicosus (Blackberry) Leaf | |
| Extract | |
| SymSitive 1609 | 1 |
| Pentylene Glycol, 4-t-Butylcyclohexanol | |
| Tegosoft TN | 4 |
| C12-15 Alkyl Benzoate | |
| Avenanthramide L | 0.001 |
| Avenanthramide A, B and C ≥100 | 2.0 |
| ppm in sum in | |
| glycerine/water | |
| Tetrasodium Glutamate Diacetate | 0.05 |
| Phytic Acid | 0.05 |
| Water | ad 100 |
| Aqua | |

TABLE 15
night cream, w/o

| Ingredients | INCI | Amount |
|---|---|---|
| Avenanthramide C | Avenanthramide C | 0.002 |
| Avenanthramide A, B | | 1.0 |
| and C ≥ 100 | | |
| ppm in sum in | | |
| glycerine/water | | |
| SymDecanox DPG | Dipropylene Glycol, | 0.5 |
| | Hydroxymethoxyphenyl | |
| | Decanone | |
| Avenanthramide B | Avenanthramide B | 0.005 |
| Aloe Vera Gel | Water (Aqua), | 3.0 |
| Concentrate 10/1 * | Aloe Barbadensis Leaf Juice | |
| Alugel 34 TH | Aluminium Stearate | 1.0 |
| Dragosan W/O P* | Sorbitan Isostearate, | 6.0 |
| | Hydrogenated Castor Oil, | |
| | Ceresin, Beeswax (Cera Alba) | |
| Dragosantol ® 100* | Bisabolol | 0.2 |
| Extrapone ® Witch | Propylene Glycol, Hamamelis | 1.0 |
| Hazel Distillate | Virginiana (Witch Hazel) Water, | |
| colourless | Water (Aqua), Hamamelis | |
| | Virginiana (Witch Hazel) Extract | |
| Perfume oil PO1, PO2, | Fragrance | 0.4 |
| PO3, PO4, or PO5 | | |
| Glycerol 85% | Glycerin | 2.0 |
| Hydrolite-5 | Pentylene Glycol | 0.5 |
| Karion F | Sorbitol | 2.0 |
| Magnesium Chloride | Magnesium Chloride | 0.7 |
| PCL Liquid 100 | Cetearyl Ethylhexoate | 12.0 |
| Retinyl Palmitate in Oil | Retinyl Palmitate | 0.2 |
| Sun Flower Oil | Helianthus Annuus (Sunflower) | 5.0 |
| | Seed Oil | |
| Sweet Almond Oil | Prunus dulcis | 5.0 |
| Sym Matrix ® | Maltodextrin, Rubus Fruticosus | 1.0 |
| | (Blackberry) Leaf Extract | |
| SymOcide PS | Phenoxyethanol, Decylene | 0.5 |
| | glycol, 1,2-Hexanediol | |
| SymVital ® AgeRepair | Zingiber Officinale (Ginger) | 0.1 |
| | Root Extract | |
| Tocopherol Acetate | Tocopheryl Acetate | 3.0 |
| Water (demineralized) | Water (Aqua) | ad 100 |

TABLE 16
Body lotion

| Ingredients | Amount |
|---|---|
| Cetearyl Alcohol | 2.0 |
| Ethylhexyl Isononanoate | 5.0 |
| Cetearyl Ethylhexanoate, Isopropyl Myristate | 3.0 |
| Glyceryl Oleate Citrate, Caprylic/Capric Triglyceride | 4.0 |
| Water (Aqua) | ad 100 |
| Pentylene Glycol | 3.0 |
| Carbomer | 0.3 |
| Sodium Benzoate | 0.5 |
| Propylene Glycol | 5.0 |
| Sodium Hydroxide | 0.3 |
| (30% solution) | |
| Perfume oil PO1, PO2, PO3, PO4, or PO5 | 0.3 |
| 4-Hydroxyacetophenone | 0.1 |
| Disodium EDTA | 0.1 |
| Avenanthramide L | 0.02 |
| Avenanthramide B | 0.02 |

TABLE 17
Antibacterial body lotion, sprayable

| Ingredients | INCI | Amount |
|---|---|---|
| Natrlquest E30 | Trisodium Ethylendiamine | 0.05 |
| | Disuccinate | |
| SymSave H | 4-Hydroxyacetophenone | 0.05 |
| Avenanthramide C | Avenanthramide C | 0.0005 |
| Triethyl Citrate | Triethyl Citrate | 0.2 |
| 2,4-Hexadienoic acid, | Sorbic acid, potassium salt | 0.5 |
| potassium salt | | |
| Dow Corning 345 Fluid | Cyclomethicone | 0.5 |
| Dracorin ® GOC | Glyceryl Oleate Citrate, | 2.0 |
| | Caprylic/Capric Triglyceride | |
| Drago-Calm | Water, Glycerin, Avena Sativa | 1.0 |
| | (Oat) Kernel Extract | |
| Dragosantol ® 100* | Bisabolol | 0.1 |
| Perfume oil PO1, PO2, | Fragrance | 0.3 |
| PO3, PO4, or PO5 | | |
| Hydrolite ®-5 | Pentylene Glycol | 5.0 |
| Neutral Oil | Caprylic/Capric Triglyceride | 4.0 |
| Paraffin Oil | Mineral Oil | 4.0 |
| PCL Liquid 100 | Cetearyl Ethylhexoate | 7.0 |
| Pemulen TR-2 | Acrylates/C10-30 Alkyl Acrylate | 0.2 |
| | Crosspolymer | |
| Sodium Hydroxide | Sodium Hydroxide | 0.4 |
| (10% solution) | | |
| SymDeo ® MPP | Dimethyl Phenylbutanol | 0.5 |
| SymRelief ® 100 | Bisabolol, Zingiber Officinale | 0.1 |
| | (Ginger) Root Extract | |
| Water (demineralized) | Water (Aqua) | ad 100 |

TABLE 18
Aseptic wound cream

| Ingredients | Amount |
|---|---|
| Sorbitan Isostearate, Hydrogenated Castor Oil, Ceresin, | 6.0 |
| Beeswax (Cera Alba) | |
| Petrolatum | 21.0 |
| Cera Alba | 5.0 |
| Cetearyl Alcohol | 7.0 |
| Prunus Dulcis | 7.0 |
| Lanolin | 5.0 |
| Paraffinum Liquidum | 12.0 |
| Perfume oil PO1, PO2, PO3, PO4, or PO5 | 0.3 |
| Water (Aqua) | ad 100 |
| Panthenol | 7.0 |
| Magnesium Sulfate | 0.7 |
| Pentylene Glycol (Hydrolite-5 Green) | 1.0 |
| Tocopheryl Acetate | 1.0 |
| Octenidine dihydrochloride | 0.1 |
| Phenoxyethanol | 0.5 |
| Phytic Acid | 0.05 |
| Disodium EDTA (Trilon BD) | 0.05 |
| Avenanthramide C | 0.001 |
| 4-Hydroxyacetophenone (SymSave H) | 0.5 |

TABLE 19
Anti acne balm

| Ingredients | INCI | Amount |
|---|---|---|
| Avenanthramide L | Avenanthramide L | 0.0005 |
| Avenanthramide C | Avenanthramide C | 0.005 |
| EDTA BD | Disodium EDTA | 0.05 |
| SymDecanox DPG | Dipropylene Glycol, | 0.5 |
| | Hydroxymethoxyphenyl | |
| | Decanone | |
| SymSave H | 4-Hydroxyacetophenone | 0.5 |
| Abil 350 | Dimethicone | 1.0 |
| Allantoin | Allantoin | 0.1 |
| Aloe Vera Gel | Water (Aqua), Aloe | 3.0 |
| Concentrate 10/1 * | Barbadensis Leaf Juice | |
| Azelaic Acid | Azelaic Acid | 5.0 |
| Cetiol OE | Dicaprylyl Ether | 4.0 |
| Cetiol SB 45 | Butyrospermum Parkii (Shea | 1.0 |
| | Butter) | |
| D-Panthenol | Panthenol | 1.0 |
| SymClariol | Decylene Glycol | 0.1 |
| Emulsiphos ® | Potassium Cetyl Phosphate, | 2.0 |
| | Hydrogenated Palm Glycerides | |
| Perfume oil PO1, PO2, | Fragrance | 0.2 |
| PO3, PO4, or PO5 | | |
| Frescolat ®ML cryst, | Menthyl Lactate | 0.8 |
| Glycerol 85% | Glycerin | 4.0 |
| Hydroviton ® PLUS | Water, Pentylene Glycol, | 1.0 |
| | Glycerin, Fructose, Urea, Citric | |
| | Acid, Sodium Hydroxide, | |
| | Maltose, Sodium PCA, Sodium | |
| | Chloride, Sodium Lactate, | |
| | Trehalose, Allantoin, Sodium | |
| | hyaluronate, Glucose | |
| Lara Care A-200 | Galactoarabinan | 0.3 |
| Pemulen TR-2 | Acrylates/C10-30 Alkyl Acrylate | 0.2 |
| | Crosspolymer | |
| Sodium Hydroxide | Sodium Hydroxide | 0.4 |
| (10% solution) | | |
| SymTriol | Caprylyl glycol, 1,2-Hexanediol, | 1.0 |
| | Methylbenzyl alcohol | |
| Tegosoft TN | C12-15 Alkyl Benzoate | 5.0 |
| Tocopherol Acetate | Tocopheryl Acetate | 0.5 |
| Water (demineralized) | Water (Aqua) | ad 100 |

TABLE 20
Barrier repair cream

| Ingredients | INCI | Amount |
|---|---|---|
| Avenanthramide L | Avenanthramide L | 0.001 |
| Avenanthramide C | Avenanthramide C | 0.003 |
| EDTA BD | Disodium EDTA | 0.05 |
| Citric Acid | Citric Acid | 0.05 |
| SymSave H | 4-Hydroxyacetophenone | 0.6 |
| Abil 350 | Dimethicone | 0.5 |
| Allantoin | Allantoin | 0.25 |
| Ceramide BIO* | Cetylhydroxyproline | 0.5 |
| | Palmitamide | |
| Dracorin ® CE | Glyceryl Stearate Citrate | 1.5 |
| Dragoxat ® 89 | Ethylhexyl Ethylisononan-oate | 2.0 |
| Emulsiphos ® | Potassium Cetyl Phosphate, | 2.0 |
| | Hydrogenated Palm Glycerides | |
| Extrapone ® Rosemary | Glycerin, Water (Aqua), | 0.5 |
| GW | Rosmarinus officinalis | |
| | (Rosemary) Leaf Extract | |
| Perfume oil PO1, PO2, | Fragrance | 0.1 |
| PO3, PO4, or PO5 | | |
| Glycerol 85% | Glycerin | 3.0 |
| Glyceryl Stearate | Glyceryl Stearate | 2.0 |
| Hydroviton ® 24 | Water, Glycerin, Sodium | 1.0 |
| | Lactate, TEA Lactate, Serine, | |
| | Lactic Acid, Urea, Sorbitol, | |
| | Sodium Chloride, Lauryl | |
| | Diethylenediaminoglycine, | |
| | Lauryl Aminopropyl-glycine, | |
| | Allantoin | |
| Hydrolite- 5 Green | Pentylene Glycol | 1.0 |
| Isodragol ® | Triisononanoin | 3.0 |
| Lanette O | Cetearyl Alcohol | 2.0 |
| NaOH | Sodium Hydroxide | 0.3 |
| (10% solution) | | |
| Neutral Oil | Caprylic/Capric Triglyceride | 10.0 |
| SymCalmin ® | Pentylene Glycol, Butylene | 1.0 |
| | Glycol, Hydroxyphenyl | |
| | Propamidobenzoic Acid | |
| Sym Repair ® 100 | Hexyldecanol, Bisabolol, | 2.0 |
| | Cetylhydroxyproline | |
| | Palmitamide, Stearic Acid, | |
| | Brassica Campestris | |
| | (Rapeseed) Sterols | |
| SymTriol | Caprylyl glycol, 1,2-Hexanediol, | 1.0 |
| | Methylbenzyl alcohol | |
| Tegosoft PC 31 | Polyglyceryl 3-Caprate | 0.3 |
| Tocopherol Acetate | Tocopheryl Acetate | 0.3 |
| Water (demineralized) | Water (Aqua) | ad 100 |

TABLE 21
Skin soothing lotion

| Ingredients | INCI | Amount |
|---|---|---|
| Avenanthramide L | Avenanthramide L | 0.001 |
| SymSave H | 4-Hydroxyacetophenone | 0.3 |
| Avenanthramide C | Avenanthramide A | 0.01 |
| Natrlquest E30 | Trisodium Ethylenediamine | 0.05 |
| | Disuccinate | |
| Phytic Acid | Phytic Acid | 0.05 |
| Abil 350 | Dimethicone | 2.0 |
| Allantoin | Allantoin | 0.2 |
| Carbopol Ultrez-10 | Carbomer | 0.1 |
| Ceramide BIO* | Cetylhydroxyproline | 0.1 |
| | Palmitamide | |
| Citric Acid | Citric Acid | 0.4 |
| (10% solution) | | |
| Emulsiphos ® | Potassium Cetyl Phosphate, | 2.0 |
| | Hydrogenated Palm Glycerides | |
| Extrapone ® Green Tea | Glycerin, Water (Aqua), | 0.2 |
| GW | Camellia Sinensis Leaf Extract | |
| Extrapone ® Rosemary | Glycerin, Water (Aqua), | 0.3 |
| GW | Rosmarinus officinalis | |
| | (Rosemary) Leaf Extract | |
| Perfume oil PO1, PO2, | Fragrance | 0.3 |
| PO3, PO4, or PO5 | | |
| Glycerol 85% | Glycerin | 2.0 |
| Glyceryl Stearate | Glyceryl Stearate | 2.0 |
| Isodragol ® | Triisononanoin | 2.0 |
| Keltrol RD | Xanthan Gum | 0.1 |
| Lanette O | Cetearyl Alcohol | 3.0 |
| Neo PCL wssl, N | Trideceth-9, PEG-5 | 1.0 |
| | Ethylhexanoate, Water | |
| PCL Liquid 100 | Cetearyl Ethylhexanoate | 5.0 |
| PCL Solid | Stearyl Heptanoate, Stearyl | 2.0 |
| | Caprylate | |
| Propylene Glycol | Propylene Glycol | 5.0 |
| Sodium Hydroxide | Sodium Hydroxide | 0.3 |
| (10% solution.) | | |
| SymCalmin ® | Pentylene Glycol, Butylene | 2.0 |
| | Glycol, Hydroxyphenyl | |
| | Propamidobenzoic Acid | |
| SymMatrix ® | Maltodextrin, Rubus Fruticosus | 0.1 |
| | (Blackberry) Leaf Extract | |
| 2-Phenoxyethyl Alcohol | Phenoxyethanol | 0.4 |
| SymSitive ®1609 | Pentylene Glycol, 4-t- | 1.5 |
| | Butylcyclohexanol | |
| Water (demineralized) | Water (Aqua) | ad 100 |

TABLE 22
Baby nappy rash cream, w/o

| Ingredients | Amount |
|---|---|
| SymOcide PH | 1 |
| Phenoxyethanol, 4-hydroxyacetophenone, Caprylyl | |
| Glycol, Water (Aqua) | |
| Cupuaçu butter | 1 |
| Theobroma Grandiflorum Seed Butter | |
| Cutina HR Powder | 1.5 |
| Hydrogenated Castor Oil | |
| Dehymuls PGPH | 5 |
| Polyglyceryl-2 Dipolyhydroxystearate | |
| Glycerin | 5 |
| Glycerin | |
| Jojoba oil | 5 |
| Simmondsia Chinensis (Jojoba) Seed Oil | |
| Magnesium Sulfate Hepta Hydrate | 0.5 |
| Magnesium Sulfate | |
| Monomuls 90-018 | 1 |
| Glyceryl Oleate | |
| Neutral oil | 8 |
| Caprylic/capric triglyceride | |
| PCL Liquid 100 | 5 |
| Cetearyl Ethylhexanoate | |
| SymCalmin | 1 |
| Butylene Glycol, Pentylene Glycol, Hydroxyphenyl | |
| Propamidobenzoic Acid | |
| Tamanu oil | 0.2 |
| Calophyllum Inophyllum Seed Oil | |
| Tetrasodium EDTA | 0.1 |
| Tetrasodium EDTA | |
| Titan dioxide | 4 |
| Titan dioxide | |
| Water | ad 100 |
| Aqua | |
| Wheat germ oil | 2 |
| Triticum Vulgare (Wheat) Germ Oil | |
| Zinc oxide | 10 |
| Zinc oxide | |
| SymSave H (4-hydroxyacetophenone) | 0.5 |
| Avenanthramide L | 0.0003 |
| Avenanthramide A | 0.002 |

TABLE 22
Skin lightening day cream, o/w

| Ingredients | INCI | Amount |
|---|---|---|
| Avenanthramide L | Avenanthramide L | 0.001 |
| Avenanthramide B | Avenanthramide B | 0.002 |
| Avenanthramide A | Avenanthramide A | 0.002 |
| SymSave H | 4-hydroxyacetophenone | 0.3 |
| Natrlquest E30 | Trisodium Ethylendiamine | 0.1 |
| | Disuccinate | |
| Abil 350 | Dimethicone | 0.5 |
| Dracorin ® CE | Glyceryl Stearate Citrate | 2.5 |
| Dracorin ® GOC | Glyceryl Oleate Citrate, | 0.5 |
| | Caprylic/Capric Triglyceride | |
| Drago-Beta-Glucan | Water (Aqua), Butylene Glycol, | 0.3 |
| | Glycerin, Avena Sativa (Oat), | |
| | Kernel Extract | |
| Dragosantol ® 100* | Bisabolol | 0.2 |
| Perfume oil PO1, | Fragrance | 0.1 |
| PO2, PO3, PO4, or | | |
| PO5 | | |
| Frescolat ®MGA | Menthone Glycerol Acetal | 0.5 |
| Glycerol 85% | Glycerin | 3.0 |
| Isopropyl Palmitate | Isopropyl Palmitate | 4.0 |
| Keltrol RD | Xanthan Gum | 0.2 |
| Lanette 16 | Cetyl Alcohol | 1.0 |
| Neo Heliopan ® AV | Ethylhexyl Methoxy-cinnamate | 5.0 |
| Neutral Oil | Caprylic/Capric Triglyceride | 6.0 |
| PCL Liquid 100 | Cetearyl Ethylhexoate | 3.0 |
| Sodium Benzoate | Sodium Benzoate | 0.1 |
| Symdiol ®68T | 1,2-Hexanediol, Caprylylglycol, | 0.5 |
| | Tropolone | |
| SymVital ® | Zingiber Officinale (Ginger) Root | 0.1 |
| AgeRepair | Extract | |
| SymWhite ®377 | Phenylethyl Resorcinol | 0.5 |
| Water | Water (Aqua) | ad 100 |
| (demineralized) | | |

TABLE 24
Shampoo

| | Amount |
|---|---|
| Antil 127 | 0.5 |
| PEG-120 Methyl Glucose Dioleate | |
| Brazilian nut oil | 0.5 |
| Bertholletia Excelsa Seed Oil | |
| Cocamidopropyl Betaine 38% | 5 |
| Cocamidopropyl Betaine | |
| Octopirox | 0.3 |
| Piroctone olamine | |
| Dragoderm | 0.5 |
| Glycerin. Triticum Vulgare Gluten. Aqua | |
| Fragrance | 0.5 |
| Perfum | |
| Glycerin | 0.5 |
| Glycerin | |
| Jojoba oil | 0.5 |
| Simmondsia Chinensis (Jojoba) Seed Oil | |
| Marlinat 242/90 M | 15 |
| MIPA Laureth Sulfate. Propylene Glycol | |
| Marlowet CG | 2 |
| PEG-18 Castor Oil Dioleate | |
| Plantacare 1200 UP | 0.5 |
| Lauryl Glucoside | |
| Polyquaternium-10 | 0.3 |
| Polyquaternium-10 | |
| Sodium Chloride | 1.5 |
| Sodium Chloride | |
| SymCalmin | 1 |
| Butylene Glycol. Pentylene Glycol. Hydroxyphenyl | |
| Propamidobenzoic Acid | |
| SymOcide PS | 0.8 |
| Phenoxyethanol. Decylene Glycol.1,2-Hexanediol | |
| Avenanthramide L | 0.0001 |
| Avenanthramide C | 0.002 |
| Disodium EDTA | 0.1 |
| SymSave H (4-hydroxyacetophenone) | 0.5 |
| Water | ad 100 |
| Aqua | |

TABLE 25
Anti dandruff shampoo

| Ingredients | INCI | Amount |
|---|---|---|
| Avenanthramide L | Avenanthramide L | 0.001 |
| Avenanthramide A | Avenanthramide A | 0.15 |
| Avenanthramide B | Avenanthramide B | 0.05 |
| Vitamin C | Ascorbic Acid | 0.03 |
| EDTA BD | Disodium EDTA | 0.05 |
| SymSave H | Hdroxyacetophenone | 1.0 |
| Aloe Vera Gel | Water (Aqua), Aloe | 0.5 |
| Concentrate 10/1 * | Barbadensis Leaf Juice | |
| Abrasive/Exfoliant | Perlite | 0.3 |
| Cellulose fibre | Microcrystalline Cellulose | 0.1 |
| Avocado oil | Persea Gratissima (Avocado) | 0.5 |
| | Oil | |
| Citric Acid 10% sol. | Citric Acid | 0.3 |
| Comperlan 100 | Cocamide MEA | 0.5 |
| Crinipan AD | Climbazole | 0.2 |
| Dragoderm ® | Glycerin, Triticum Vulgare | 2.0 |
| | (Wheat) Gluten, Water (Aqua) | |
| Perfume oil PO1, PO2, | Fragrance | 0.5 |
| PO3, PO4, or PO5 | | |
| Genapol LRO liquid | Sodium Laureth Sulfate | 37.0 |
| Merquat 550 | Polyquaternium-7 | 0.5 |
| Xylityl Caprylate | Xylityl Caprylate | 0.5 |
| Sodium Chloride | Sodium Chloride | 1.0 |
| Hydrolite-5 Green | Pentylene Glycol | 0.5 |
| Tego Betain L7 | Cocamidopropyl Betaine | 6.0 |
| Water (demineralized) | Water (Aqua) | ad 100 |

TABLE 26
2-in-1 Shampoo

| Ingredients | INCI Name | Amount |
|---|---|---|
| Deionized water | Water | ad 100 |
| Shea butter | Butyrospermum Parkii (Shea) | 0.1 |
| | Butter | |
| SymSave H | 4-Hydroxyacetophenone | 0.5 |
| SymDiol 68 | 1.2 Hexanediol, Caprylyl | 0.5 |
| | Glycol | |
| Plantacare PS 10 | Sodium Laureth Sulfate, Lauryl | 20.0 |
| | Glucoside | |
| Euperlan PK 771 | Glycol Distearate, Sodium | 6.0 |
| | Lauryl Sulfate, Cocamide | |
| | MEA, Laureth-10 | |
| Sodium chloride | Sodium Chloride | 1.4 |
| Citric acid | Citric acid | 0.1 |
| monohydrate | | |
| crystalline | | |
| Perfume oil PO1, PO2, | Fragrance | 0.5 |
| PO3, PO4, or PO5 | | |
| Zinc Omadine | Zinc pyrithione | 0.10 |
| Dissolvine GL47 | Tetrasodium Glutamate | 0.1 |
| | Diacetate | |
| Phytic Acid | Phytic Acid | 0.1 |
| Avenanthramide L | Avenanthramide L | 0.001 |
| Avenanthramide B | Avenanthramide B | 0.002 |

TABLE 27
Body wash

| Ingredients | INCI | Amount |
|---|---|---|
| Lumerol K 28 | Disodium Laureth | 33.0 |
| | Sulfosuccinate, | |
| | Cocamidopropyl Betaine, | |
| | Magnesium Lauryl Sulfate | |
| Amphotensid B 4 | Cocamidopropyl Betaine | 10.0 |
| Pearly Gloss | MIPA-Pareth-25 Sulfate, | 4.0 |
| | Glycol Stearate | |
| Sodium Chloride | Sodium Chloride | 2.0 |
| Avocado oil | Persea Gratissima (Avocado) | 3.0 |
| | Oil | |
| SymSave H | 4-Hydroxyacetophenone | 0.8 |
| Hydrolite-5 Green | Pentylene Glycol | 1.0 |
| Water | Water | ad 100 |
| Perfume oil PO1, PO2, | Fragrance | 0.5 |
| PO3, PO4, or PO5 | | |
| Symlite G8 | Glyceryl caprylate | 0.15 |
| DragoCalm | Water (Aqua), Glycerin. Avena | 2.0 |
| | Sativa (Oat) Kernel Extract | |
| Citric Acid | Citric Acid | 0.1 |
| EDTA BD | Disodium EDTA | 0.1 |

TABLE 28
Shower gel

| Ingredients | INCI | Amount |
|---|---|---|
| Deionized water | Water | ad 100 |
| Shea butter | Butyrospermum Parkii (Shea) | 1.0 |
| | Butter | |
| Plantacare PS 10 | Sodium Laureth Sulfate, Lauryl | 20.0 |
| | Glucoside | |
| Hydrolite-6 | 1.2 Hexanediol | 0.5 |
| Dehydroacetic acid | Dehydroacetic acid | 0.2 |
| SymSave H | 4-Hydroxyacetophenone | 0.3 |
| Sodium chloride | Sodium Chloride | 1.4 |
| Citric acid | Citric Acid | 1.3 |
| monohydrate | | |
| crystalline | | |
| Perfume oil PO1, PO2, | Fragrance | 0.6 |
| PO3, PO4, or PO5 | | |
| Dissolvine GL47 | Tetrasodium Glutamate | 0.1 |
| | Diacetate | |
| Avenanthramide C | Avenanthramide L | 0.01 |
| Symlite G 8 | Glyceryl caprylate | 0.3 |
| Avenanthramide L | Avenanthramide A | 0.02 |

TABLE 29
Intimate wash

| Ingredients | INCI | Amount |
|---|---|---|
| Tegobetaine HS | Cocamidopropyl Betaine, | 15.0 |
| | Glyceryl Laurate | |
| Tagat L 2 | PEG-20 Glyceryl Laurate | 2.0 |
| Arlacide G | Chlorhexidine Digluconate | 0.1 |
| Rewoquat B 50 | Benzalkonium Chloride | 0.1 |
| Lactic Acid. 80% | Lactic Acid | 0.1 |
| euxyl ® K700 | Potassium Sorbate, Benzyl | 0.3 |
| | Alcohol. Phenoxyethanol | |
| Water | Water | ad 100 |
| Perfume oil PO1, PO2, | Fragrance | 0.2 |
| PO3, PO4, or PO5 | | |
| Hydrolite-5 Green | Pentylene Glycol | 0.3 |
| SymSave H | 4-Hydroxyacetophenone | 0.3 |
| Lactic Acid | Lactic Acid | 0.1 |
| EDTA BD | Disodium EDTA | 0.1 |
| Avenanthramide L | Avenanthramide L | 0.001 |
| Avenanthramide C | Avenanthramide C | 0.002 |

TABLE 30
Syndet soap, liquid

| Ingredients | INCI | Amount |
|---|---|---|
| Elfan OS 46 | Sodium Olefin C14-C16 | 35.5 |
| | Sulfonate | |
| Armoteric LB | Lauryl Betaine | 8.0 |
| Euperlan PK 3000 OK | Glycol Distearate, Glycerin, | 10.0 |
| | Laureth-4, Cocamidopropyl | |
| | Betaine | |
| Elfacos GT 282 L | Talloweth-60 Myristyl Glycol | 3.0 |
| PCL-Liquid 100 | Cetearyl Ethylhexanoate | 4.0 |
| Perfume oil PO1, PO2, | Fragrance | 0.4 |
| PO3, PO4, or PO5 | | |
| SymSave H | 4-Hydroxyacetophenone | 0.6 |
| Avenanthramide L | Avenanthramide L | 0.05 |
| Avenanthramide A | Avenanthramide A | 0.5 |
| Water | Water | ad 100 |

TABLE 31
Anti-acne wash

| Ingredients | Amount |
|---|---|
| Water (Aqua) | ad 100 |
| Polyquaternium-7 | 0.5 |
| Cocam idopropyl Betaine | 9.0 |
| Coco Glucoside | 2.0 |
| Polysorbate 80, Glycerol. Gossypium Herbaceum | 1.0 |
| (Cotton) Seed Oil, Water (Aqua) | |
| Trideceth-9, PEG-5 Ethylhexanoate, Water (Aqua) | 1.0 |
| Glycereth-90 Isostearate, Laureth-2 | 0.5 |
| Sodium Laureth Sulfate | 37.0 |
| Glycerol. Triticum Vulgare (Wheat) Gluten. Water (Aqua) | 1.0 |
| Sodium Chloride | 0.3 |
| Perfume oil PO1, PO2, PO3, PO4, or PO5 | 1.0 |
| SymOcide BHO (4-hydroxyacetophenone, Benzyl alcohol, | 0.3 |
| Caprylyl glycol, Water) | |
| SymSave H (4-hydroxyacetophenone) | 0.8 |
| Avenanthramide L | 0.25 |
| Avenanthramide A | 0.15 |

TABLE 32
After Shave Tonic

| Ingredients | INCI | Amount |
|---|---|---|
| SymSol ® PF-3 | Water (Aqua). Pentylene | 3.0 |
| | Glycol. Sodium Lauryl | |
| | Sulfoacetate, Sodium Oleoyl | |
| | Sarcosinate, Sodium Chloride, | |
| | Disodium Sulfoacetate, | |
| | SodiumOleate, Sodium | |
| | Sulfate | |
| SymSitive ® 1609 | Pentylene Glycol, 4-t- | 1.0 |
| | Butylcyclohexanol | |
| Frescolat ® ML | Menthyl Lactate | 0.3 |
| Glycerol 99.5 P. | Glycerol | 5.0 |
| Water | Water (Aqua) | ad 100 |
| Extrapone ® Glacier | Glycerol, Water (Aqua) | 1.0 |
| Water GW | | |
| SymCalmin ® | Butylene Glycol, Pentylene | 0.5 |
| | Glycol, Hydroxyphenyl | |
| | Propamidobenzoic Acid | |
| Dragosine ® | Carnosine | 0.1 |
| Hydrolite ® 5 | Pentylene Glycol | 5.0 |
| Ethanol 96% | Alcohol Denat. | 5.0 |
| Colour Pigment | Colour Pigment | 0.05 |
| Perfume oil PO1, PO2, | Fragrance | 0.15 |
| PO3, PO4, or PO5 | | |
| SymSave H | 4-hydroxyacetophenone | 0.8 |
| Avenanthramide L | Avenanthramide L | 0.001 |
| Avenanthramide A | Avenanthramide A | 0.002 |

TABLE 33
Scalp soothing hair conditioner with
UV-B/UV-A protection, rinse off

| Ingredients | INCI | Amount |
|---|---|---|
| Avenanthramide L | Avenanthramide L | 0.0005 |
| SymSave H | 4-Hydroxyacetophenone | 0.5 |
| Avenanthramide C | Avenanthramide C | 0.002 |
| Vitamin C | Ascorbic Acid | 0.1 |
| EDTA BD | Disodium EDTA | 0.1 |
| Abil 350 | Dimethicone | 0.1 |
| Dehyquart A CA | Cetrimonium Chloride | 0.5 |
| Dehyquart SP | Quaternium-52 | 4.0 |
| Dracorin ® CE | Glyceryl Stearate Citrate | 1.0 |
| EDETA BD | Disodium EDTA | 0.1 |
| Extrapone ® Green Tea | Glycerin, Water (Aqua), | 0.7 |
| GW | Camellia Sinensis Leaf Extract | |
| Perfume oil PO1, PO2, | Fragrance | 0.5 |
| PO3, PO4, or PO5 | | |
| Lara Care A-200 | Galactoarabinan | 0.5 |
| Neutral Oil | Caprylic/Capric Triglyceride | 1.0 |
| PCL Liquid 100 | Cetearyl Ethylhexoate | 0.3 |
| PCL Solid | Stearyl Heptanoate, Stearyl | 3.0 |
| | Caprylate | |
| SymOcide ®PS | Phenoxyethanol, Decylene | 0.5 |
| | Glycol, 1,2-Hexanediol | |
| Water (demineralized) | Water (Aqua) | ad 100 |

TABLE 34
Hair conditioner, leave on

| Ingredients | INCI | Amount |
|---|---|---|
| Avenanthramide L | Avenanthramide L | 0.015 |
| SymSave H | 4-Hydroxyacetophenone | 0.3 |
| Avenanthramide A | Avenanthramide A | 0.02 |
| Dehyquart A CA | Cetrimonium Chloride | 0.2 |
| Dehyquart SP | Quaternium-52 | 2.0 |
| Dracorin ® CE | Glyceryl Stearate Citrate | 1.0 |
| Drago-Calm | Water, Glycerin, Avena Sativa | 2.0 |
| | (Oat) Kernel Extract | |
| Dissolvine GL47 | Tetrasodium Glutamate | 0.1 |
| | Diacetate | |
| Citric Acid | Citric Acid | 0.1 |
| Farnesol | Farnesol | |
| Perfume oil PO1, PO2, | Fragrance | 0.5 |
| PO3, PO4, or PO5 | | |
| Lara Care A-200 | Galactoarabinan | 0.1 |
| Polymer JR 400 | Polyquaternium-10 | 0.1 |
| Propylene Glycol | Propylene Glycol | 0.8 |
| SymMollient ® WS | Trideceth-9. PEG-5 | 1.0 |
| | Isononanoate. Water | |
| SymSol ®PF3 | Water, Pentylene Glycol, | 1.5 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| SymTriol ® | Caprylyl Glycol, 1.2- | 0.6 |
| | Hexanediol, Methylbenzyl | |
| | Alcohol | |
| Water (demineralized) | Water (Aqua) | ad 100 |

TABLE 35
Deodorant stick

| Ingredients | Amount |
|---|---|
| Sodium stearate | 8.0 |
| PPG-3 Myristyl ether | 70.0 |
| 1.2-propylene glycol | 10.0 |
| 1.1-dimethyl-3-phenylpropanol | 0.2 |
| 2-butyloctanoic acid | 0.2 |
| Perfume oil PO1, PO2, PO3, PO4, or PO5 | 0.6 |
| Water | ad 100 |
| SymDeo Plus | 0.5 |
| (Jasmol (2-benzlheptanol), 1-Dodecanol (Lauryl Alcohol), | |
| 1,2-Decanediol (Decylene Glycol), 2-Phenoxyethyl | |
| Alcohol (Phenoxyethanol)) | |
| Avenanthramide L | 0.001 |
| Ascorbic Acid | 0.1 |
| Tetrasodium Glutamate Diacetate (Dissolvine GL 47) | 0.1 |
| SymSave H (4-hydroxyacetophenone) | 0.4 |
| Avenanthramide C | 0.001 |

TABLE 36
Antiperspirant/deodorant roll-on

| Ingredients | INCI | Amount |
|---|---|---|
| Avenanthramide L | Avenanthramide L | 0.001 |
| Vitamin C | Ascorbic Acid | 0.01 |
| EDTA BD | Disodium EDTA | 0.1 |
| SymSave H | 4-Hydroxyacetophenone | 0.5 |
| Avenanthramide B | Avenanthramide A | 0.004 |
| Dragosantol ® 100* | Bisabolol | 0.1 |
| Ethanol 96% | Ethanol | 30.0 |
| Farnesol | Farnesol | 0.5 |
| Perfume oil PO1, PO2, | Fragrance | 1.5 |
| PO3, PO4, or PO5 | | |
| Frescolat ®ML cryst, | Menthyl Lactate | 0.2 |
| Irgasan DP 300 | Triclosan | 0.3 |
| Natrosol 250 HHR | Hydroxyethyl-cellulose | 0.3 |
| Solubilizer 611674 | PEG-40 Hydrogenated Castor | 2.0 |
| | Oil, Trideceth-9, Water (Aqua) | |
| SymDeo ® B125 | 2-Methyl 5-Cyclohexylpentanol | 0.5 |
| Water (demineralized) | Water (Aqua) | ad 100 |
| Zirkonal L 450 | Aluminium Zirconium | 37.0 |
| | Pentachloro-hydrate | |
| | (40% aqueous solution) | |

TABLE 37
Deodorant formulation in the form of a roll-on gel

| Ingredients | Amount |
|---|---|
| 1.3-butylene glycol | 2.0 |
| 2-Methyl 5-cyclohexylpentanol | 0.1 |
| PEG-40-hydrogenated castor oil | 2.0 |
| Hydroxyethylcellulose | 0.5 |
| Pentylene Glycol | 1.0 |
| Perfume oil PO1, PO2, PO3, PO4, or PO5 | 0.3 |
| 1,3-propanediol | 0.5 |
| SymGuard CD (3-Phenylpropanol, o-cymen-3-ol, | 0.4 |
| Decylene glycol) | |
| Ethylhexyl glycerin | 0.1 |
| Disodium EDTA | 0.05 |
| Malic Acid | 0.05 |
| SymSave H (4-hydroxyacetophenone) | 0.5 |
| Avenanthramide C | 0.001 |
| Avenanthramide L | 0.004 |
| Water | ad 100 |

TABLE 38
Clear deo anti-perspirant roll-on

| Ingredients | INCI | Amount |
|---|---|---|
| Methocel E4M Premium | Hydroxypropyl Methylcellulose | 0.5 |
| Water | Water (Aqua) | ad 100 |
| Neo-PCL Water Soluble | Trideceth-9, PEG-5 | 1.0 |
| N | Ethylhexanoate, Water (Aqua) | |
| Solubilizer | PEG-40 Hydrogenated Castor | 3.0 |
| | Oil, Trideceth-9, Propylene | |
| | Glycol, Water (Aqua) | |
| Deolite | Dimethyl Phenylpropanol, | 0.5 |
| | Pentylene Glycol | |
| Locron LW | Aluminium Chlorohydrate | 25.0 |
| Aloe Vera Gel | Aloe Barbadensis Leaf Juice | 1.0 |
| Concentrate 10/1 | | |
| 1.2-Propylene Glycol 99 | Propylene Glycol | 4.0 |
| P GC | | |
| Ethanol 96% | Alcohol Denat. | 30.0 |
| Perfume oil PO1, PO2, | Fragrance | 1.0 |
| PO3, PO4, or PO5 | | |
| SymSave H | 4-Hydroxyacetophenone | 0.5 |
| Phytic Acid | Phytic Acid | 0.1 |
| Avenanthramide A | Avenanthramide A | 0.005 |
| Avenanthramide L | Avenanthramide L | 0.001 |
| Avenanthramide C | Avenanthramide C | 0.005 |

TABLE 39
Deodorant pump spray with SymClariol

| Ingredients | INCI | Amount |
|---|---|---|
| SymClariol ® | Decylene Glycol | 0.2 |
| Solubilizer | PEG-40 Hydrogenated Castor | 4.0 |
| | Oil, Trideceth-9, Propylene | |
| | Glycol, Water (Aqua) | |
| Neo-PCL Water Soluble | Trideceth-9, PEG-5 | 1.5 |
| N | Ethylhexanoate, Aqua | |
| SymRelief ® | Bisabolol, Zingiber Officinale | 0.1 |
| | (Ginger) Root Extract | |
| Water | Water (Aqua) | ad 100 |
| 1,2-Propylene Glycol | Propylene Glycol | 6.0 |
| Perfume oil PO1, PO2, | Perfume | 0.4 |
| PO3, PO4, or PO5 | | |
| SymDiol ® 68 | 1,2-Hexanediol, Caprylyl Glycol | 0.2 |
| Vitamin C | Ascorbic Acid | 0.025 |
| EDTA BD | Disodium EDTA | 0.2 |
| SymCalmin | Butylene Glycol, Pentylene | 2.0 |
| | Glycol, Hydroxyphenyl | |
| | Propamidobenzoic Acid | |

TABLE 40
Whitening deodorant spray

| Ingredients | Amount |
|---|---|
| PEG-40-hydrogenated castor oil | 3.0 |
| Ethylhexylglycerol (Octoxyglycerol) | 0.2 |
| Symbright 2036 (Sclareolide) | 0.1 |
| Ethanol | 40.0 |
| Citrate buffer | 0.5 |
| 1.2-Hexanediol, 1.2-Octanediol (SymDiol 68) | 0.3 |
| SymOcide C (o-cymen-5-ol) | 0.05 |
| 2-Benzylheptan-1-ol (Jasmol) | 0.1 |
| Perfume oil PO1, PO2, PO3, PO4, or PO5 | 0.75 |
| Disodium EDTA (EDTA BD) | 0.1 |
| Phenoxyethanol | 0.4 |
| Avenanthramide C | 0.001 |
| SymSave H (4-hydroxyacetophenone) | 0.5 |
| Water | ad 100 |

TABLE 41
Sunscreen lotion (o/w, broadband protection)

| Ingredients | INCI | Amount |
|---|---|---|
| Avenanthramide C | Avenanthramide C | 0.005 |
| SymSave H | 4-Hydroxyacetophenone | 0.3 |
| Citric Acid | Citric Acid | 0.1 |
| Avenanthramide B | Avenanthramide B | 0.005 |
| Carbopol Ultrez-10 | Carbomer | 0.2 |
| Dow Corning 246 Fluid | Cyclohexasiloxane and | 2.0 |
| | Cyclopentasiloxane | |
| Dragosantol ® 100* | Bisabolol | 0.3 |
| EDETA BD | Disodium EDTA | 0.1 |
| Emulsiphos ® | Potassium Cetyl Phosphate, | 1.5 |
| | Hydrogenated Palm Glycerides | |
| Perfume oil PO1, PO2, | Fragrance | 0.4 |
| PO3, PO4, or PO5 | | |
| Frescolat ®MGA | Menthone Glycerol Acetal | 0.3 |
| Glycerol 85% | Glycerin | 4.7 |
| Keltrol RD | Xanthan Gum | 0.2 |
| Lanette O | Cetearyl Alcohol | 1.0 |
| Neo Heliopan ® 357 | Butyl Methoxy-dibenzoyl- | 1.0 |
| | methane | |
| Neo Heliopan ® AP | Disodium Phenyl | 10.0 |
| (10% as sodium salt) | Dibenzimidazole Tetrasulfonate | |
| Neo Heliopan ® AV | Ethylhexyl Methoxy-cinnamate | 3.0 |
| Neo Heliopan ® Hydro | Phenylbenz-imidazole Sulfonic | 6.7 |
| (15% as sodium salt) | Acid | |
| Neo Heliopan ® MBC | 4-Methylbenzyl-idene Camphor | 1.5 |
| Neo Heliopan ® OS | Ethylhexyl Salicylate | 5.0 |
| Neutral Oil | Caprylic/Capric Triglyceride | 2.0 |
| SymMatrix ® | Maltodextrin, Rubus Fruticosus | 0.3 |
| | (Blackberry) Leaf Extract | |
| SymOcide ® BHO | 4-Hydroxyacetophenone, | 1.5 |
| | Benzyl alcohol, Caprylyl glycol, | |
| | Aqua | |
| Tegosoft TN | C12-15 Alkyl Benzoate | 5.0 |
| Tocopherol Acetate | Tocopheryl Acetate | 0.5 |
| Triethanolamine, 99% | Triethanolamine | 0.5 |
| Water (demineralized) | Water (Aqua) | ad 100 |

TABLE 42
Sun protection milk, w/o

| Ingredients | INCI | Amount |
|---|---|---|
| Dehymuls PGPH | Polyglyceryl-2 | 3.0 |
| | dipolyhydroxystearate | |
| Beeswax 8100 | Beeswax | 1.0 |
| Monomuls 90-0-18 | Glyceryl oleate | 1.0 |
| Zinc stearate | Zinc stearate | 1.0 |
| Hydrolite-8 | Caprylyl Glycol | 0.3 |
| Cetiol SN | Cetearyl isononanoate | 5.0 |
| Cetiol OE | Dicaprylyl ether | 5.0 |
| Tegosoft TN | C12-15 alkyl benzoate | 4.0 |
| Vitamin E | Tocopherol | 0.5 |
| Neo Heliopan ® OS | Ethylhexyl salicylate | 5.0 |
| Neo Heliopan ® AV | Ethylhexyl methoxycinnamate | 7.5 |
| Uvinul ® T150 | Ethylhexyl triazone | 1.5 |
| Water. distilled | Water (Aqua) | ad 100 |
| Trilon BD | Disodium EDTA | 0.1 |
| Glycerol | Glycerol | 5.0 |
| Neo Heliopan ® AP | Disodium phenyl | 15.0 |
| 10% solution. | dibenzimidazole tetrasulfonate | |
| neutralized with NaOH | | |
| Perfume oil PO1, | Fragrance | 0.25 |
| PO2, PO3, PO4, or | | |
| PO5 | | |
| Dragosantol 100 | Bisabolol | 0.1 |
| Phytic Acid | Phytic Acid | 0.05 |
| SymSave H | 4-Hydroxyacetophenone | 0.8 |
| Avenanthramide A | Avenanthramide A | 0.002 |
| Avenanthramide B | Avenanthramide B | 0.002 |

TABLE 43
Sun spray with UV-A/B-broadband protection with low oil content

| Ingredients | INCI | Amount |
|---|---|---|
| EDTA BD | Disodium EDTA | 0.05 |
| Vitamin C | Ascorbic Acid | 0.025 |
| Avenanthramide L | Avenanthramide L | 0.001 |
| SymCalmin | Pentylene Glycol, Butylene | 2.0 |
| | Glycol, Hydroxyphenyl | |
| | Propamidobenzoic Acid | |
| SymSave H | 4-Hydroxyacetophenone | 0.5 |
| Avenanthramide C | Avenanthramide C | 0.002 |
| | | 0.05 |
| Ethanol 96% | Ethanol | 13.0 |
| Perfume oil PO1, PO2, | Fragrance | 0.5 |
| PO3, PO4, or PO5 | | |
| Glyceryl Stearate | Glyceryl Stearate | 4.0 |
| Hydroviton ® PLUS | Water, Pentylene Glycol, | 1.0 |
| | Glycerin, Fructose, Urea, Citric | |
| | Acid, Sodium Hydroxide, | |
| | Maltose, Sodium PCA, Sodium | |
| | Chloride, Sodium Lactate, | |
| | Trehalose, Allantoin, Sodium | |
| | hyaluronate, Glucose | |
| Isoadipate ® | Diisopropyl Adipate | 1.0 |
| Neo Heliopan ® AV | Ethylhexyl Methoxy-cinnamate | 25.0 |
| Neo Heliopan ® MBC | 4-Methylbenzyl-idene Camphor | 33.3 |
| Propylene Glycol | Propylene Glycol | 0.8 |
| Tego Betain L7 | Cocamidopropyl Betaine | 1.0 |
| Water (demineralized) | Water (Aqua) | ad 100 |

TABLE 44
Sunscreen spray, o/w (SPF 15-20)

| Ingredients | INCI | Amount |
|---|---|---|
| Dracorin ® GOC | Glyceryl Oleate Citrate, | 2.0 |
| | Caprylic/Capric Triglyceride | |
| Corapan ® TQ | Diethylhexyl 2,6-Naphthalate | 3.0 |
| Neo Heliopan ® HMS | Homosalate | 7.0 |
| Neo Heliopan ® OS | Ethylhexyl Salicylate | 5.0 |
| Neo Heliopan ® 357 | Butyl Methoxydibenzoylmethane | 3.0 |
| Isoadipate | Diisopropyl Adipate | 6.0 |
| Baysilone ® Oil M10 | Dimethicone | 1.0 |
| Edeta ® BD | Disodium EDTA | 0.1 |
| Vitamin E Acetate | Tocopheryl Acetate | 0.5 |
| Dragosantol ® 100 | Bisabolol | 0.1 |
| Pemulen ® TR-2 | Acrylates/C10-30 Alkyl Acrylate | 0.25 |
| | Crosspolymer | |
| Water | Water (Aqua) | ad 100 |
| Glycerol 99.5 P, | Glycerol | 4.0 |
| Butylene Glycol | Butylene Glycol | 5.0 |
| Neo Heliopan ® Hydro | Phenylbenzimidazole Sulfonic | 8.0 |
| (103089), used as 25% | Acid | |
| aq, solution neutralized | | |
| with Biotive ® L-Arginine | | |
| Biotive ® L-Arginine | Arginine | 0.55 |
| Perfume oil PO1, PO2, | Fragrance | 0.4 |
| PO3, PO4, or PO5 | | |
| SymOcide PS | Phenoxyethanol, 1,2-Hexanediol, | 0.5 |
| | Decylene glycol | |
| SymSave H | 4-Hydroxyacetophenone | 0.5 |
| Vitamin C | Ascorbic Acid | 0.05 |
| Avenanthramide B | Avenanthramide B | 0.005 |
| Avenanthramide L | Avenanthramide L | 0.05 |
| Avenanthramide A | Avenanthramide A | 0.05 |

TABLE 45
After sun gel

| Ingredients | INCI | Amount |
|---|---|---|
| SymSol ® PF-3 | Water (Aqua), Pentylene Glycol, | 3.0 |
| | Sodium Lauryl Sulfoacetate, | |
| | Sodium Oleoyl Sarcosinate, | |
| | Sodium Chloride, Disodium | |
| | Sulfoacetate, Sodium Oleate, | |
| | Sodium Sulfate | |
| Glycerol 99.5 P | Glycerol | 5.0 |
| SymHelios ® 1031 | Benzylidene | 0.1 |
| | Dimethoxydimethylin danone | |
| Water | Water (Aqua) | ad 100 |
| Pemulen ® TR-2 | Acrylates/C10-30 Alkyl Acrylate | 1.0 |
| | Crosspolymer | |
| D-Panthenol 75 W | Panthenol | 0.5 |
| SymFinity ® 1298 | Echinacea Purpurea Extract | 0.1 |
| Extrapone ® Pearl GW | Water (Aqua), Glycerol, | 1.0 |
| | Hydrolyzed Pearl, Xanthan Gum | |
| Sodium Hydroxide | Sodium Hydroxide | 2.5 |
| (10% solution) | | |
| Ethanol 96% | Alcohol Denat, | 15.0 |
| Perfume oil PO1, PO2, | Fragrance | 0.2 |
| PO3, PO4, or PO5 | | |
| SymOcide ® PS | Phenoxyethanol, 1,2- | 0.8 |
| | Hexanediol, Decyleneglycol | |
| Phytic Acid | Phytic Acid | 0.05 |
| Dissolvine GL47 | Tetrasodium Glutamate | 0.05 |
| | Diacetate | |
| SymSave H | 4-Hydroxyacetophenone | 0.5 |
| Avenanthramide C | Avenanthramide C | 0.0025 |

TABLE 46
After sun lotion

| Ingredients | Amount |
|---|---|
| Acrylate/C10-30 alkylacrylate crosspolymer | 0.4 |
| Cetearylethyl hexanoate | 15.0 |
| Bisabolol | 0.2 |
| Tocopheryl acetate | 1.0 |
| Panthenol | 1.0 |
| Alcohol | 15.0 |
| Glycerol | 3.0 |
| Perfume oil PO1, PO2, PO3, PO4, or PO5 | 0.30 |
| 1.2-Hexanediol (Hydrolite-6) | 1.0 |
| Triethanolamine | 0.2 |
| Pentylene glycol (Hydrolite-5 Green) | 4.0 |
| Aqua dem. | ad 100 |
| 4-Hydroxyacetophenone (SymSave H) | 0.3 |
| Ascorbic Acid (Vitamin C) | 0.05 |
| Disodium EDTA (EDTA BD) | 0.05 |
| Pentylene Glycol, Butylene Glcol, Hydroxyphenyl | 2.0 |
| Propamidobenzoic Acid (SymCalmin) | |
| Avenanthramide A | 0.005 |

TABLE 47
Syndet antimicrobial soap bar

| Ingredients | INCI | Amount |
|---|---|---|
| Zetesap 813 A | Disodium Lauryl | ad 100 |
| | Sulfosuccinate, Sodium Lauryl | |
| | Sulfate, Corn Starch, Cetearyl | |
| | Alcohol, Paraffin, Titanium | |
| | Dioxide | |
| Amphotensid GB 2009 | Disodium | 6.0 |
| | Cocoamphodiacetate | |
| Allantoin | Allantoin | 1.0 |
| Perfume oil PO1, PO2, | Fragrance | 1.0 |
| PO3, PO4, or PO5 | | |
| SymOcide C | o-cymen-5-ol | 0.1 |
| SymSave H | 4-Hydroxyacetophenone | 0.5 |
| Vitamin C | Ascorbic Acid | 0.05 |
| EDTA BD | Disodium EDTA | 0.05 |
| Avenanthramide L | Avenanthramide L | 0.001 |
| Avenanthramide B | Avenanthramide B | 0.005 |
| Avenanthramide C | Avenanthramide A | 0.005 |

TABLE 48
Syndet soap bar

| Ingredients | INCI | Amount |
|---|---|---|
| Fenopon AC-78 | Sodium Cocoyl Isethionate | 20.0 |
| Natrium laurylsulfoacetate | Sodium Lauryl Sulfoacetate | 16.0 |
| Paraffin | Paraffin | 19.0 |
| Wax. microcrystalline | Microcrystalline Wax | 1.0 |
| Corn Starch | Corn Starch | 8.0 |
| Coconut acid | Coconut acid | 2.0 |
| Lauric acid diethanol | Lauramide DEA | 2.0 |
| amide | | |
| Dextrin | Dextrin | 21.0 |
| Lactic acid, 88% | Lactic Acid | 0.05 |
| Citric Acid | Citric Acid | 0.2 |
| EDTA BD | Disodium EDTA | 0.05 |
| SymGuard CD | 3-Phenylpropanol, o-cymen- | 0.3 |
| | 5-ol, Decylene glycol | |
| Thymol | Thymol | 0.05 |
| Symlite G8 | Glyceryl Caprylate | 0.2 |
| Water | Water | ad 100 |
| Perfume oil PO1, PO2, | Fragrance | 1.0 |
| PO3, PO4, or PO5 | | |
| Avenanthramide C | Avenanthramide L | 0.001 |
| SymSave H | 4-Hydroxyacetophenone | 0.5 |
| Avenanthramide A | Avenanthramide A | 0.003 |

TABLE 49
Shaving foam

| Ingredients | Amount |
|---|---|
| Dem. Water | ad 100 |
| Triethanolamine | 4.0 |
| Edenor L2 SM (Stearinic acid, Palmitinic acid) (Cognis) | 5.3 |
| Laureth-23 | 3.0 |
| Stearylalcohol | 0.5 |
| Avenanthramide L | 0.001 |
| SymSave H (4-hydroxyacetophenone) | 0.3 |
| Malic Acid | 0.05 |
| EDTA BD (Disodium EDTA) | 0.05 |
| Dihydroavenanthramide D | 0.05 |
| Sodium lauryl sulfate | 3.0 |
| Extrapone Seaweed (Water, Propylene glycol, | 1.0 |
| Potassium iodide, Fucus Vesiculosus Extract) | |
| Dragosantol (Bisabolol, Farnesol) | 0.1 |
| Perfume oil PO1, PO2, PO3, PO4, or PO5 | 1.0 |
| Propane, butane 4,2 Bar | 4.0 |

TABLE 50
Sprayable disinfecting gel

| Ingredients | INCI | Amount |
|---|---|---|
| Water | Water (Aqua) | ad 100 |
| Stabileze QM | PVM/Ma Decadiene | 0.25 |
| | Crosspolymer | |
| Sodium Hydroxide | Sodium Hydroxide | 0.4 |
| (10% solution) | | |
| Coffein pure | Caffeine | 0 . . . 5 |
| Extrapone ® Horse | Propylene Glycol, Water (Aqua), | 1.0 |
| Chestnut | Glucose, Aesculus | |
| | Hippocastanum (Horse | |
| | Chestnut) Seed Extract, Lactic | |
| | Acid | |
| Hydrolite ® 5 | Pentylene Glycol | 3.0 |
| 1,3 Butylene Glycol | Butylene Glycol | 5.0 |
| Biotive ® Esculin | Esculin | 0.3 |
| Sesquihydrate | | |
| Ethanol 96% | Alcohol Denat, | 10.0 |
| Solubilizer | PEG-40 Hydrogenated Castor | 0.5 |
| | Oil, Trideceth-9, Water (Aqua) | |
| Perfume oil PO1, PO2, | Fragrance | 0.2 |
| PO3, PO4, or PO5 | | |
| Octenidine | Octenidine dihydrochloride | 0.1 |
| dihydrochloride | | |
| Phenoxyethanol | Phenoxyethanol | 0.5 |
| Phytic Acid | Phytic Acid | 0.05 |
| Dissolvine GL 47 | Tetrasodium Glutamate | 0.05 |
| | Diacetate | |
| Avenanthramide L | Avenanthramide L | 0.0005 |
| Avenanthramide C | Avenanthramide A | 0.003 |
| Avenanthramide B | Avenanthramide B | 0.002 |

TABLE 51
Solution for wet wipes

| Ingredients | INCI | Amount |
|---|---|---|
| SymSol ® PF-3 | Water (Aqua), Pentylene Glycol, | 2.0 |
| | Sodium Lauryl Sulfoacetate, | |
| | SodiumOleoyl Sarcosinate, | |
| | Sodium Chloride, Disodium | |
| | Sulfoacetate, SodiumOleate, | |
| | Sodium Sulfate | |
| Dragosantol ® 100 | Bisabolol | 0.1 |
| Glycerol 99.5 P, | Glycerol | 5.0 |
| Water | Water (Aqua) | ad 100 |
| Hydrolite ® 5 | Pentylene Glycol | 5.0 |
| D-Panthenol 75 W | Panthenol | 0.8 |
| Witch Hazel-Distillate | Hamamelis Virginiana (Witch | 1.0 |
| | Hazel) Water, Water (Aqua), | |
| | Alcohol | |
| Allplant Essence ® | Pelargonium Graveolens | 1.0 |
| Org, Rose Geranium P | Flower/Leaf/Stem Water | |
| Perfume oil PO1, PO2, | Fragrance | 0.1 |
| PO3, PO4, or PO5 | | |
| Avenanthramide L | Avenanthramide L | 0.2 |
| Avenanthramide A, B | Water (Aqua), Glycerin, Avena | 2.0 |
| and C ≥ 100 | Sativa (Oat) Kernel Extract | |
| ppm in sum in | | |
| glycerine/water | | |
| Vitamin C | Ascorbic Acid | 0.05 |
| SymDecanox DPG | Dipropylene Glycol, | 1.0 |
| | Hydroxymethoxyphenyl | |
| | Decanone | |

TABLE 52
Further preferred cleansing formulations without sodium lauryl ether sulfate (SLES) (% (w/w)).

| INCI | 1 | 2 | 3 | 4 | 5 | 6 | 7 | 8 | 9 | 10 |
|---|---|---|---|---|---|---|---|---|---|---|
| Avenanthramide L | — | 0.001 | — | — | 0.001 | — | — | 0.0005 | 0.2 | — |
| Avenanthramide C | — | — | — | 0.005 | 0.005 | — | — | 0.002 | 0.1 | — |
| Avenanthramide A | — | — | — | 0.005 | 0.005 | — | — | 0.002 | — | — |
| Avenanthramide A, B | 1.0 | — | 0.5 | — | — | 1.5 | 1.0 | — | — | 2.0 |
| and C ≥100 | | | | | | | | | | |
| ppm in sum in | | | | | | | | | | |
| glycerine/water | | | | | | | | | | |
| 4-Hydroxyacetophenone | 0.5 | 0.6 | 0.3 | 0.8 | 0.2 | 0.5 | 0.5 | 0.5 | 0.3 | 0.1 |
| (SymSave H) | | | | | | | | | | |
| Ascorbic Acid | 0.025 | 0.01 | — | — | — | — | — | — | — | — |
| Citric Acid | — | — | — | — | — | 0.05 | 0.05 | 0.05 | 0.05 | 0.05 |
| Phytic Acid | — | — | 0.05- | 0.05 | — | — | — | — | — | — |
| Tetrasodium | — | — | — | — | 0.05 | 0.1 | 0.05 | — | — | — |
| Glutamate Diacetate | | | | | | | | | | |
| (Dissolvine GL47 S) | | | | | | | | | | |
| EDTA BD | 0.05 | 0.02 | 0.05 | 0.05 | — | — | — | 0.05 | 0.1 | 0.05 |
| (Disodium EDTA) | | | | | | | | | | |
| SymDecanox DPG | — | — | 0.5 | 0.5 | 0.5 | — | — | 0.5 | — | — |
| (Dipropylene Glycol, | | | | | | | | | | |
| Hydroxymethoxyphenyl | | | | | | | | | | |
| Decanone | | | | | | | | | | |
| 1.2 Hexanediol, | 0.5 | 0.5 | — | — | 1.0 | 0.5 | — | 0.5 | 0.5 | — |
| Caprylyl Glycol | | | | | | | | | | |
| (Symdiol 68) | | | | | | | | | | |
| 1,2 Hexanediol, | — | — | 0.7 | — | — | — | 0.7 | — | — | — |
| Caprylyl Glycol, | | | | | | | | | | |
| Tropolone (Symdiol 68 | | | | | | | | | | |
| T) | | | | | | | | | | |
| Ammonium Lauryl | — | 5.0 | — | — | — | — | — | — | — | — |
| Sulfate | | | | | | | | | | |
| (Stepanol AM) | | | | | | | | | | |
| Aqua, Glycerin, | — | — | — | — | — | 0.3 | — | — | — | — |
| Echinacea Purpurea | | | | | | | | | | |
| Extract (Extrapone | | | | | | | | | | |
| Echinacea) | | | | | | | | | | |
| Aqua, Pentylene | — | — | — | — | — | 3.0 | — | — | — | — |
| Glycol, Sodium Lauryl | | | | | | | | | | |
| Sulfoacetate, Sodium | | | | | | | | | | |
| Oleoyl Sarcosinate, | | | | | | | | | | |
| Sodium Chloride, | | | | | | | | | | |
| Sodium Oleate | | | | | | | | | | |
| (SymSol PF3) | | | | | | | | | | |
| Bisabolol (Dragosantol | — | — | — | — | 0.1 | — | — | | — | — |
| 100) | | | | | | | | | | |
| Butyrospermum Parkii | | | | | | | | | | 13.0 |
| Butter | | | | | | | | | | |
| (Cetiol SB 45) | | | | | | | | | | |
| Caprylic/Capric | — | — | — | — | — | — | — | — | — | 2.0 |
| Triglyceride | | | | | | | | | | |
| Hydroxymethoxyphenyl | | | | | | | | | | |
| Decanone | | | | | | | | | | |
| (Symdecanox HA) | | | | | | | | | | |
| Caprylyl Glycol, 1,2 | — | — | — | — | 0.8 | — | — | — | 0.8 | — |
| Hexanediol, | | | | | | | | | | |
| Methylbenzyl Alcohol | | | | | | | | | | |
| Citric Acid 30% | — | — | — | 3.0 | — | — | — | | — | — |
| aqueous sol. | | | | | | | | | | |
| Climbazole (Crinipan | — | — | — | — | — | — | — | 0.5 | — | — |
| AD) | | | | | | | | | | |
| Cocamide MEA | — | — | — | — | — | — | — | 3.0 | — | — |
| (Mackamide CMA) | | | | | | | | | | |
| Cocamide MIPA | 0.5 | — | — | — | — | — | — | — | — | — |
| Cocoamidopropyl | 15.0 | 5.0 | 3.0 | 6.0 | 14.0 | — | — | 15.0 | 17.0 | — |
| Betaine | | | | | | | | | | |
| (Tego Betain F50) | | | | | | | | | | |
| Coco Betaine (Dehyton | — | — | — | — | — | — | 2.0 | — | — | — |
| AB 30) | | | | | | | | | | |
| Coco-Glucoside | — | 10.0 | — | — | — | — | — | — | — | — |
| (Plantacare 818 UP) | | | | | | | | | | |
| Decyl Glycoside | — | — | — | — | 2.0 | — | — | — | — | — |
| (Ecosense 3000) | | | | | | | | | | |
| Disodium Cocoyl | — | 3.0 | — | — | — | — | — | — | — | — |
| Glutamate (Plantapon | | | | | | | | | | |
| ACG LC) | | | | | | | | | | |
| Disodium Lauryl | — | — | 2.0 | 2.0 | — | — | — | — | — | — |
| Sulfosuccinate | | | | | | | | | | |
| (Setacin F spezial) | | | | | | | | | | |
| Fragrance PO1, PO2, | 1.0 | 0.5 | 0.1 | 0.3 | 0.1 | 0.3 | 0.5 | 0.3 | 0.05 | — |
| PO3, PO4, or PO5 | | | | | | | | | | |
| Glycerin 99% | — | — | 0.5 | — | 3.0 | — | — | — | — | — |
| Glycerin, Aqua, | — | — | — | — | 1.0 | — | — | — | — | — |
| Hamamelis Virginia | | | | | | | | | | |
| Bark/Leaf/Twig Extract | | | | | | | | | | |
| (Extrapone Witch | | | | | | | | | | |
| Hazel GW) | | | | | | | | | | |
| Glyceryl Caprylate | 0.1 | 0.5 | 0.2 | 0.3 | 0.5 | 0.1 | — | | — | — |
| (Symlite G8) | | | | | | | | | | |
| Glycol Distearate, | — | — | — | 2.0 | — | — | — | 3.0 | — | — |
| Laureth-4, | | | | | | | | | | |
| Cocoamidopropyl | | | | | | | | | | |
| Betaine (Quickpearl | | | | | | | | | | |
| PK3) | | | | | | | | | | |
| Isostearamide MIPA, | — | — | — | 1.0 | — | — | — | | — | — |
| Glyceryl Laurate (Antil | | | | | | | | | | |
| SPA 80) | | | | | | | | | | |
| Kaolin | — | — | — | — | — | — | — | — | — | 18.8 |
| (ImerCare ® 02K-S) | | | | | | | | | | |
| Lactic Acid 90% | — | — | — | — | 0.3 | — | — | | — | — |
| aqueous sol. | | | | | | | | | | |
| Lauroyl/Myristoyl | 12.0 | — | — | — | — | — | — | | — | — |
| Methyl Glucamide | | | | | | | | | | |
| (Glucotain Clean) | | | | | | | | | | |
| Lauryl Hydroxysultaine | — | — | — | — | — | — | 11.0 | | — | — |
| (45% AS) | | | | | | | | | | |
| Lauryl Lactate | — | — | — | — | — | — | 0.3 | | — | — |
| (Schercemol LL Ester) | | | | | | | | | | |
| Maltodextrin, | — | — | — | — | — | — | — | — | — | 0.5 |
| Lactobacillus Ferment | | | | | | | | | | |
| (SymReboot L19) | | | | | | | | | | |
| Menthyl Lactate | — | — | — | — | — | — | — | 0.2 | — | — |
| (Frescolat ML) | | | | | | | | | | |
| PEG-200 | — | — | — | 4.5 | — | — | — | — | — | — |
| Hydrogenated Glyceryl | | | | | | | | | | |
| Palmate, PEG-7 | | | | | | | | | | |
| Glyceryl Cocoate | | | | | | | | | | |
| (Antil 200) | | | | | | | | | | |
| PEG-4 | — | — | — | — | — | — | 2.0 | — | 2.8 | — |
| Rapeseedamide (92% | | | | | | | | | | |
| AS) | | | | | | | | | | |
| PEG-40 Hydrogenated | — | — | — | — | 1.5 | — | — | 0.9 | — | — |
| Castor Oil, Trideceth-9, | | | | | | | | | | |
| Propylene Glycol, | | | | | | | | | | |
| Aqua (Solubilizer | | | | | | | | | | |
| Symrise) | | | | | | | | | | |
| PEG-45 M | — | — | — | — | — | — | — | 0.15 | — | — |
| (PolyoxWSR N 60K) | | | | | | | | | | |
| Pentylene Glycol | — | — | — | — | — | 2.0 | — | — | — | 1.5 |
| (Hydrolite-5 Green) | | | | | | | | | | |
| Pentylene Glycol, 4-t- | — | — | — | — | — | 1.0 | — | — | — | — |
| Butylcyclohexanol | | | | | | | | | | |
| (Symsitive 1609) | | | | | | | | | | |
| Pentylene Glycol, | — | — | — | — | 1.0 | — | — | 2.0 | — | — |
| Butylene Glycol, | | | | | | | | | | |
| Hydroxyphenyl | | | | | | | | | | |
| Propamidobenzoic | | | | | | | | | | |
| Acid (SymCalmin) | | | | | | | | | | |
| Phenyl Propanol, O- | — | — | — | — | 0.5 | — | — | — | — | — |
| Cymen-5-ol, Decylene | | | | | | | | | | |
| Glycol (Symguard CD) | | | | | | | | | | |
| Piroctone Olamine | 0.1 | — | 0.5 | — | — | — | — | — | — | — |
| (Octopirox) | | | | | | | | | | |
| Polyacrylate 33 | — | — | — | — | — | — | — | 6.5 | — | — |
| (Rheomer 33T) | | | | | | | | | | |
| Polyquaternium-10 | — | — | — | 0.3 | — | — | — | 0.2 | — | — |
| (Polymer JR 400) | | | | | | | | | | |
| Polyquaternium-7 | 0.4 | — | — | — | — | — | — | — | — | — |
| (Dehyquart CC7) | | | | | | | | | | |
| Polysilicone -19 (Abil | — | — | — | 2.0 | — | — | — | — | — | — |
| UV Quat 50) | | | | | | | | | | |
| Potassium Sorbate | — | — | 0.3 | 0.4 | — | 0.5 | — | — | — | 0.3 |
| Propylene Glycol | — | — | — | — | 3.0 | — | — | — | — | — |
| Rhamnose | — | — | — | — | — | 0.5 | — | — | — | — |
| Sodium C14-C16 | — | — | — | — | — | — | 27.0 | — | — | — |
| Olefin Sulfonate (38% | | | | | | | | | | |
| AS) | | | | | | | | | | |
| Sodium Chloride | 0.5 | — | — | | — | — | — | — | — | — |
| Sodium | — | — | — | 6.0 | — | — | — | — | — | — |
| Cocoamphoacetate | | | | | | | | | | |
| (Rewoteric AMC) | | | | | | | | | | |
| Sodium Cocoyl | — | — | — | — | — | 2.0 | — | — | — | — |
| Alaninate | | | | | | | | | | |
| (Amilite ACS 12) | | | | | | | | | | |
| Sodium Cocoyl | — | 5.0 | 3.0 | — | — | — | — | — | — | — |
| Glutamate (Hostapon | | | | | | | | | | |
| CCG) | | | | | | | | | | |
| Sodium Cocoyl | 10.0 | — | — | — | — | — | — | — | — | — |
| Glycinate (Hostapon | | | | | | | | | | |
| SG) | | | | | | | | | | |
| Sodium Cocoyl | 4.5 | — | — | — | — | — | — | — | — | 15.0 |
| Isethionate (ELFAN ® | | | | | | | | | | |
| AT 84) | | | | | | | | | | |
| Sodium Hydroxide | — | — | — | — | — | — | — | 0.5 | — | — |
| (50% solution) | | | | | | | | | | |
| Sodium Laureth-5 | — | — | — | — | — | — | — | — | 8.0 | — |
| Carboxylate | | | | | | | | | | |
| (Akypo Foam RL 40) | | | | | | | | | | |
| Sodium Laureth-6 | — | — | — | — | — | 1.0 | — | — | — | — |
| Carboxylate | | | | | | | | | | |
| (Akypo SOFT 45 HP) | | | | | | | | | | |
| Sodium Lauroyl | 3.0 | — | — | — | — | — | — | — | — | — |
| Glutamate (Hostapon | | | | | | | | | | |
| CLG) | | | | | | | | | | |
| Sodium Lauroyl | — | — | — | — | 2.0 | — | — | — | — | — |
| Lactylate (Dermosoft | | | | | | | | | | |
| SLL) | | | | | | | | | | |
| Sodium Lauroyl Methyl | — | — | — | 22.0 | — | — | — | — | — | — |
| Isethionate (Iselux LQ- | | | | | | | | | | |
| CLR SB) | | | | | | | | | | |
| Sodium Lauroyl | — | 3.0 | — | 3.0 | — | — | — | — | — | — |
| Sarcosinate (Protelan | | | | | | | | | | |
| LS 9011) | | | | | | | | | | |
| Sodium Lauryl Glucose | — | — | — | — | — | — | — | 6.0 | — | — |
| Carboxylate | | | | | | | | | | |
| Lauryl Glucoside | | | | | | | | | | |
| (Plantapon LGC Sorb) | | | | | | | | | | |
| Sodium Myristoyl | — | — | — | — | — | — | — | — | — | 30.0 |
| Glutamate | | | | | | | | | | |
| (Amisoft MS11) | | | | | | | | | | |
| Sodium Salicylate | — | — | 0.3 | — | — | — | — | — | — | — |
| (Seboclear) | | | | | | | | | | |
| Sorbitol | 1.0 | — | — | — | — | — | — | | — | — |
| Trideceth-9, PEG-5 | — | — | — | — | — | — | — | 2.0 | — | — |
| Isononanoate, Water | | | | | | | | | | |
| (Aqua) | | | | | | | | | | |
| Water (Aqua), | — | — | 1.0 | — | | — | — | — | — | — |
| Glycerin, Tetraselmis | | | | | | | | | | |
| Suecica Extract | | | | | | | | | | |
| (SymControl Care) | | | | | | | | | | |
| Xanthan Gum (Keltrol | — | 0.5 | — | — | 0.5 | — | — | — | — | — |
| RD) | | | | | | | | | | |

| | |
|---|---|
| Water (Aqua) | Ad 100 |

1: Mild hair and body ash
2: Shampoo
3: Anti acne face wash
4: Color care shampoo
5: Feminine wash
6: Micellar water
7: Liquid soap
8: Antidandruff shampoo
9: Baby shampoo
10: Solid shampoo

TABLE 53
Gel dental cream

| Ingredients | I (%) | II (%) | III (%) |
|---|---|---|---|
| Sodium carboxymethylcellulose | 0.40 | 0.40 | 0.40 |
| Sorbitol 70%, in water | 72.00 | 72.00 | 72.00 |
| Polyethylenglycol (PEG) 1500 | 3.00 | 3.00 | 3.00 |
| Sodium saccharinate | 0.07 | 0.07 | 0.07 |
| Sodium fluoride | 0.24 | 0.24 | 0.24 |
| p-Hydroxybenzoic acid (PHB) | 0.15 | 0.15 | |
| ethylester | | | |
| SymDiol 68 | | | 0.5 |
| SymSave H | 0.2 | 0.02 | 0.25 |
| Peppermint flavor | 1.00 | 1.00 | 1.00 |
| Avenanthramide C | 0.03 | | |
| and B 1:1 (w/w) | | | |
| Avenanthramide L | | | 0.01 |
| Dianthramide B | | 0.005 | |
| Ascorbic Acid | 0.025 | 0.025 | 0.025 |
| Lactic Acid | 0.025 | 0.05 | 0.025 |
| Abrasive Silica | 11.00 | 11.00 | 11.00 |
| Thickening Silica | 6.00 | 6.00 | 6.00 |
| Sodium dodecylsulfate (SDS) | 1.40 | 1.40 | 1.40 |
| Distilled water | ad 100.00 | ad 100.00 | ad 100.00 |

TABLE 54
Ready-to-use mouthwash with fluoride

| Ingredients | I (%) | II (%) | III (%) |
|---|---|---|---|
| Ethanol | 7.00 | 7.00 | |
| Glycerin | 12.00 | 12.00 | |
| Sodium fluoride | 0.05 | 0.05 | 0.18 |
| Pluronic F-127 ® | 1.40 | 1.40 | |
| (BASF, surface active substance) | | | |
| Sodium phosphate buffer pH 7.0 | 1.10 | 1.10 | |
| Sorbic acid | 0.20 | 0.20 | |
| Sodium saccharinate | 0.10 | 0.10 | 0.10 |
| Cinnamon/menthol flavor | 0.15 | 0.15 | 0.15 |
| Avenanthramide A | | | 0.005 |
| Dianthramide B | | 0.002 | |
| Ascorbic Acid | 0.025 | 0.025 | 0.025 |
| Avenanthramide A, | 5.0 | | |
| B and C ≥ 100 ppm | | | |
| in sum in glycerine/water | | | |
| Colour | 0.01 | 0.01 | 0.01 |
| Sorbitol 70% | | | 10 |
| Cremophor RH455 | | | 1.8 |
| SymDiol 68 | | | 0.5 |
| SymSave H | 0.1 | 0.05 | 0.15 |
| Distilled water | ad 100.00 | ad 100.00 | ad 100.00 |

1. A composition comprising:
at least one avenanthramide, an analogue thereof, or an oat extract comprising at least one avenanthramide or an analogue thereof; and
at least one stabilizer selected from the group of chelating agents, organic carbonic acids having 2 to 10 carbon atoms, antioxidants, and mixtures of these.

2. The composition according to claim 1, wherein the at least one avenanthramide is selected from the group of the avenanthramides A, B, C, G, H, K, L and R and mixtures of these, or wherein the avenanthramide analogue is Dihydroavenanthramide D.

3. The composition according to claim 1, wherein an oat source of the oat extract is milled or non-milled grains of the species Avena sativa or Avena nuda or oat straw.

4. The composition according to claim 3, wherein the oat extract is an aqueous, alcoholic, aqueous-alcoholic, acetonic, or aqueous-acetonic extract, or an aqueous, alcoholic, aqueous-alcoholic, acetonic, or aqueous-acetonic extract obtained by subcritical fluid extraction.

5. The composition according to claim 1, wherein the chelating agent is selected from the group of EDTA and its salts, phytic acid, tetrasodium glutamate diacetate, trisodium ethylenediamine disuccinate, sodium citrate, potassium citrate, sodium phytate, sodium gluconate, calcium gluconate, caprylhydroxamic acid, galactaric acid, galacturonic acid, sodium metaphosphate, sodium polyitaconate, disodium etidronate and trisodium methylglycinediacetic acid.

6. The composition according to claim 1, wherein the organic carbonic acid is selected from the group of alpha-hydroxy acids, gluconic acid, glyceric acid, glycolic acid, isocitric acid, lactic acid, malic acid, citric acid, mandelic acid, azelaic acid, anisic acid, ectoin, ferulic acid, folic acid, levulinic acid, niacin, sebacic acid, salicylic acid, sorbic acid, tartaric acid, 2-hydroxyaotanoic acid, 2-hydroxydecanoic acid, salts of the foregoing, mixtures of the foregoing, and gluconolactone.

7. The composition according to claim 1, wherein the antioxidant is selected from the group of 4-hydroxyacetophenone, ascorbic acid, 6-paradol, uric acid, butylhydroxytoluol (BHT), butylhydroxyanisol (BHA), ascorbyl palmitate, ascorbyl phosphate and salts thereof, carnosine, sodium ascorbate, rutin, tocopherol, tocopheryl acetate, ubiquinone, tropolone and allantoin.

8. The composition according to claim 1, wherein the stabilizer comprises a chelating agent and an organic acid, or a chelating agent and an antioxidant, or an organic carbonic acid and an antioxidant, or a combination of a chelating agent, an organic acid, and an antioxidant.

9. The composition according to any claim 1, comprising:
0.0001 to 5.0 wt % of the at least one avenanthramide, analogue thereof, or oat extract comprising the at least one avenanthramide or an analogue thereof; and
0.02 to 0.5 wt % of the at least one stabilizer,
based on the total weight of the composition.

10. A method of skin protection, skin care, scalp protection, scalp care, hair care, nail care, or the prevention or treatment of intolerant and sensitive skin, skin irritation, skin reddening, wheals, pruritus (itching), skin aging, wrinkle formation, loss of skin volume, loss of skin elasticity, pigment spots, pigment abnormalities, or moisturizing dry skin, the method comprising administering the composition of claim 1 to a subject in need thereof.

11. A method of using the composition according to claim 1 as a medicament, the method comprising administering the composition to a subject in need thereof.

12. The method of claim 11, wherein the medicament is administered for the prevention and/or treatment of dermatological or keratological diseases or in the prevention or treatment of dermatological diseases associated with increased ROS production or in the prevention and/or treatment of cardiovascular diseases, allergic reactions, coronary heart disease, for decreasing the level of LDL cholesterol and lipids in blood serum, for reducing blood pressure, for improving sensitivity to insulin, or for enabling the control of blood glucose levels.

13. The method of claim 12, wherein the dermatological or keratological diseases are selected from the group of eczema, psoriasis, seborrhoea, dermatitis, erythema, pruritus (itching), otitis, inflammation, irritation, fibrosis, Lichen planus, Pityriasis rosea, Pityriasis versicolor, autoimmune bullous diseases, urticarial, angiodermal and allergic skin reactions, and wound healing, and/or skin diseases associated with increased ROS production selected from the group of atopic dermatitis, neurodermitis, psoriasis, rosacea, acneiform eruptions, sebostasis and xerosis.

14. A method for preparing foods, food supplements, or cosmetic, pharmaceutical or veterinary preparations comprising the step of including the composition of claim 1 in the preparation of the food, food supplement, or cosmetic, pharmaceutical, or veterinary preparation.

15. A food, food supplement, or cosmetic, pharmaceutical or veterinary preparation comprising the composition according to claim 1.

16. The food, food supplement, or cosmetic, pharmaceutical or veterinary preparation according to claim 15, further comprising one or more active substances selected from the group of skin-moisturising and moisture-retaining substances, cooling agents, osmolytes, keratological substances, nurturing substances, anti-inflammatory, antibacterial or antimycotic substances, substances having a reddening-alleviating or itch-alleviating action, lenitive substances, mixtures of the foregoing, cosmetically or pharmaceutically acceptable excipients selected from the group of antioxidants, preservatives, chelating agents, penetration enhancers, surface-active substances, emulsifiers, perfume oils, anti-foaming agents, colorants, pigments having a colouring action, thickeners, plasticisers, fats, oils, waxes, alcohols, polyols, polymers, foam stabilisers, electrolytes, organic solvents, silicone derivatives, and mixtures of these.

17. The cosmetic or pharmaceutical preparation according to claim 15 in the form of a fluid, tincture, lotion, emulsion, gel, cream, ointment, spray or shampoo.

18. A method for stabilizing an avenanthramide, an analogue thereof, or a composition comprising an avenanthramide or an analogue thereof, comprising combining the avenanthramide, analogue thereof, or composition comprising an avenanthramide or analogue thereof with a stabilizer selected from the group of chelating agents, organic carbonic acids having 2 to 10 carbon atoms, antioxidants and mixtures of these.

19. The food, food supplement, or cosmetic, pharmaceutical, or veterinary preparation of claim 15, comprising the composition in an amount of 0.0001 to 10 wt % based on the total weight of the food, food supplement, or cosmetic, pharmaceutical, or veterinary preparation.
